(19) United States
(12) Patent Application Publication
(10) Pub. No.: US 2004/0088116 A1
(43) Pub. Date: May 6, 2004

(54) Methods and systems for creating and using comprehensive and data-driven simulations of biological systems for pharmacological and industrial applications

(75) Inventors: Iya Khalil, Ithaca, NY (US); Colin Hill, Ithaca, NY (US)

Correspondence Address: Attention of: Barry Evans, Esq., c/o KRAMER LEVIN NAFTALIS & FRANKEL LLP, 919 Third Avenue, New York, NY, 10022-3852, US

(73) Assignee: Gene Network Sciences, Inc.

(21) Appl. No.: 10/439,288

(22) Filed: May 14, 2003

Related U.S. Application Data
(63) Continuation-in-part of application No. 10/287,173, filed on Nov. 4, 2002.

(51) Int. Cl. G06G 7/48; G06G 7/58; G06F 19/00; G01N 33/48; G01N 33/50

(52) U.S. Cl. 702/20; 703/11

(57) ABSTRACT

Presented herein are techniques and methodologies for creating large-scale data-driven models of biological systems and exemplary applications thereof including drug discovery and industrial applications. Exemplary embodiments include creating a core skeletal simulation (scaleable to any size) from known biological information, collecting quantitative and qualitative experimental data to constrain the simulation, creating a probable reactions database, integrating the core skeletal simulation, the database of probable reactions, and static and dynamical time course measurements to generate an ensemble of biological network structures and their corresponding molecular concentration profiles and phenotypic outcomes that approximate output of the original biological network used for prediction, and finally experimentally validating and iteratively refining the model. The invention further describes methods of taking conventional small-scale models and extending them to comprehensive large-scale models of biological systems. The methods presented further describe ways to create models of arbitrary size and complexity and various ways to incorporate data to account for missing biological information.

### CROSS REFERENCE TO RELATED APPLICATIONS

This is a continuation-in-part application of U.S. patent application Ser. No. 10/287,173, filed on Nov. 4, 2002 by Iya Khalil, et al. entitled METHODS AND SYSTEMS FOR THE IDENTIFICATION OF COMPONENTS OF MAMMALIAN BIOCHEMICAL NETWORKS AS TARGETS FOR THERAPEUTIC AGENTS, which is hereby incorporated herein by reference.

This application also claims priority to the following United States Patent Applications: “Cell Language”, ______, Inventors, US Application Serial No. ______, filed on Nov. 2, 2002 (inventors and serial number to be added by amendment when available); “Scale Free Network Inference Methods”, Colin Hill, Jeff Fox and Vipul Periwal, Inventors, US Application Serial No. ______, filed on Nov. 1, 2002 (inventors and serial number to be added by amendment when available); “Systems and Network Inference”, ______, Inventors, US Provisional Application Serial No. ______, filed on Nov. 1, 2002 (inventors and serial number to be added by amendment when available); and “Bioinformatic data mining system”, ______, Inventors, US Application Serial No. ______, filed on ______ (inventors, filing date and serial number to be added by amendment when available).

### FIELD OF THE INVENTION

The present invention relates to the graphic and mathematical modeling of biological systems and subsystems, and more particularly to the creation and use of comprehensive and data-driven simulations of biological systems for pharmacological and industrial applications.

### DETAILED DESCRIPTION OF THE INVENTION

In the description of the invention provided herein, numerous references to exemplary embodiments, processes and techniques are stated without language of exemplification. It is to be understood that any embodiment, technique, method, process or the like described herein is only exemplary in nature, and not meant to in any way limit, define or restrict the invention presented herein, which is greater than the sum of any one or more constituent parts, processes or methods used to describe it. In addition, where forms of the verb “to be” are used to describe techniques, processes, results or methods, or parts thereof, what is intended and understood by such usage is actually the compound auxiliary verb formation “can be.” Thus, if a given technique “is” used or a given result is said to occur, what is understood and intended is that the technique “can be” used or “may be” used and the result can or may occur.

What will be described herein are the details of a methodology for creating large-scale data-driven models of biological systems and exemplary applications thereof including drug discovery and industrial applications.

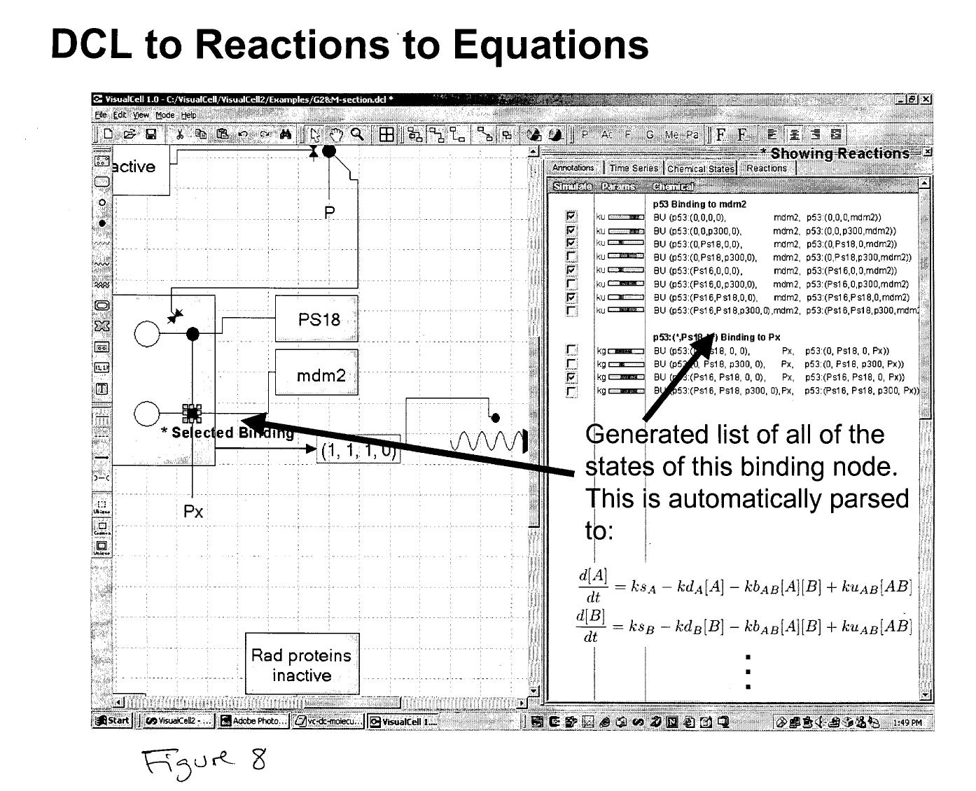

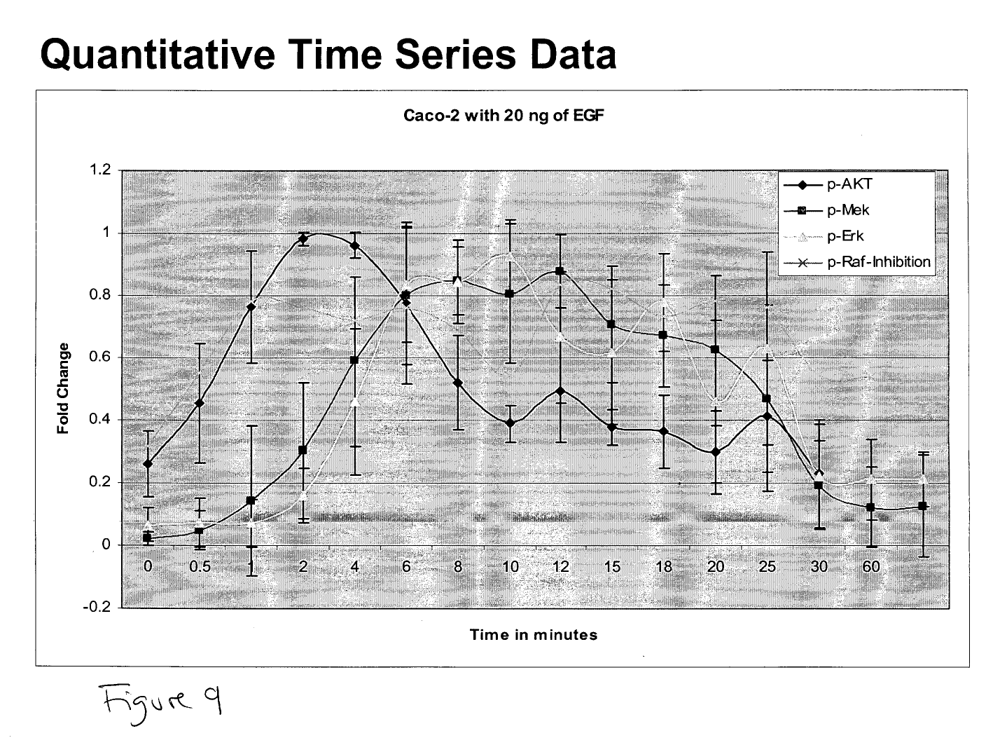

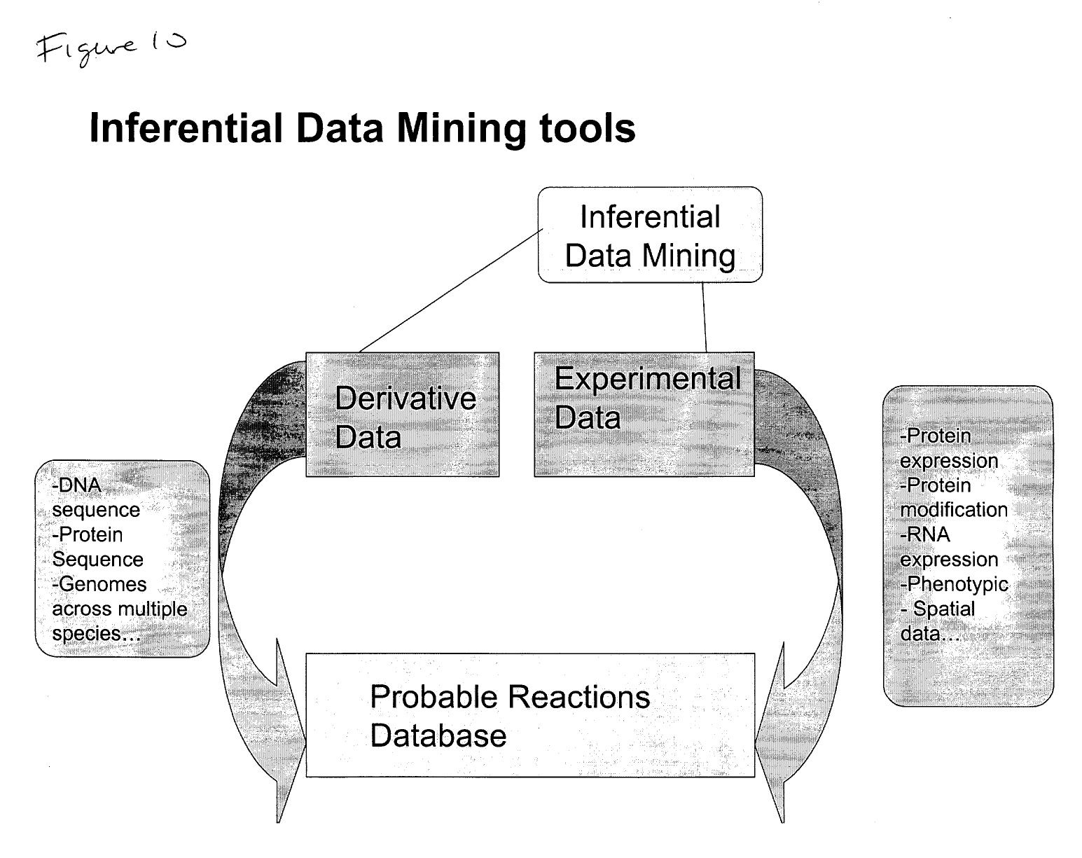

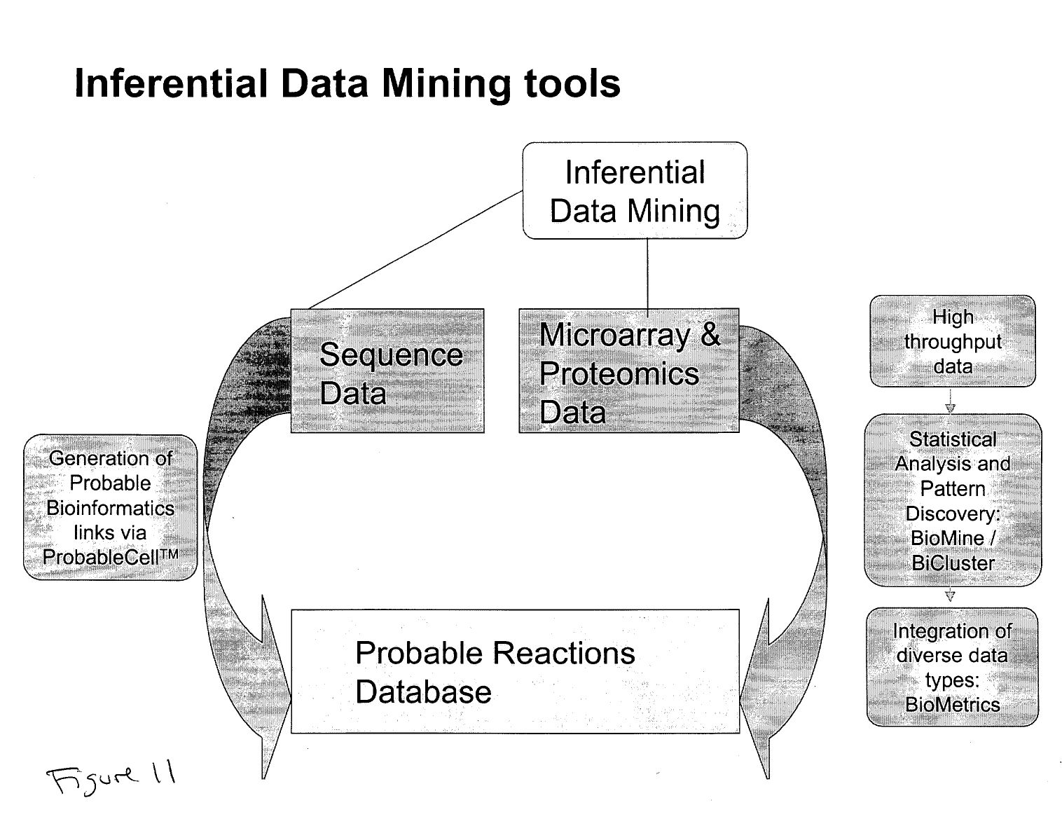

Exemplary embodiments of the present invention include (a) creating a core skeletal simulation (scaleable to any size) from known biological information; (b) collecting quantitative and qualitative experimental data to constrain the simulation (c) creating a probable reactions database; (d) integrating the core skeletal simulation, the database of probable reactions, and static and dynamical time course measurements to generate an ensemble of biological network structures and their corresponding molecular concentration profiles and phenotypic outcomes that approximate output of the original biological network used for prediction (Computational Hypothesis testing), and finally (e) experimentally validating and ititeratively refining the model. FIG. 3 contains an overview of the methodology. For each technique, the invention describes methods of taking the current state of the art applicable to developing models on a small scale and extending it to being able to model large-scale biological systems. The methods presented describe ways to create models of arbitrary size and complexity and ways to incorporate data to account for missing biological information.

Overview

The present invention is thus directed to methods for creating data-driven mathematical models of the dynamics of living cells, organisms, or biological systems that can be used to explain and predict how the individual components of such living cells, organisms, or biological systems interact to create and maintain the living system. The methods of this invention have extensive applications in the areas of drug discovery, target discovery, clinical diagnosis and treatment, genetic analysis, bioremediation, optimization of bioreactors and fermentation processes, biofilm formation, antimicrobial agents, biosensors, biodefense, and other applications involving perturbing biological systems.

The present invention includes methods that allow for the creation of large-scale predictive data-driven models of the biological system on the level of the entire genome. These methods extend beyond traditional tools conventionally used to model individual biological pathways or groups of pathways to allow the creation of models that can present every relevant gene and protein in the cell, organism, or biological system under analysis. This comprises, for example models of a few hundred genes and proteins, as represented by the smallest known organism M. genitalium, to models of systems having hundreds of thousands of genes and proteins such as are known to be present in the mammalian cell. At present, for most biological systems, information on the functionality of genes and proteins, their interactions, and the kinetics of the interactions is not known. Therefore, methods disclosed in the present invention allow for both the creation of large-scale models from known biological information and the incorporation of data from many sources to account for the inevitable missing information.

One aspect of the present invention is a methodology for creating a core simulation of a biological system that can scale to any size from known biological information. This includes, for example, (i) methods for extracting data from the vast body of public domain information, (ii) rating the biological information to determine the quality of the data, and (iii) representing the information in a concise and scalable manner that is automatically translated to a mathematical representation or set of instructions readable by a human or computer. In an exemplary embodiment, biological information is represented and modeled via the Diagrammatic Cell Language (DCL), a concise and scaleable language for representing and simulating biological interactions. Additionally, the present invention includes methods for modularizing a given biological system into discrete functional modules for ease of scalability and interpretation of simulation results. In an exemplary embodiment methods are provided to model components best described by discreet stochastic events such as the rupturing of the mitochondrial wall and connecting such events to components modeled in a continuous framework, such as proteins in a signaling cascade that initiated the event. Another exemplary embodiment describes how to connect the specific chemical output of a model to a phenotypic level for predicting cellular physiology. Two exemplary applications of the methodology are described in some detail. These are whole cell simulations of a mammalian cell and of E. Coli bacteria.

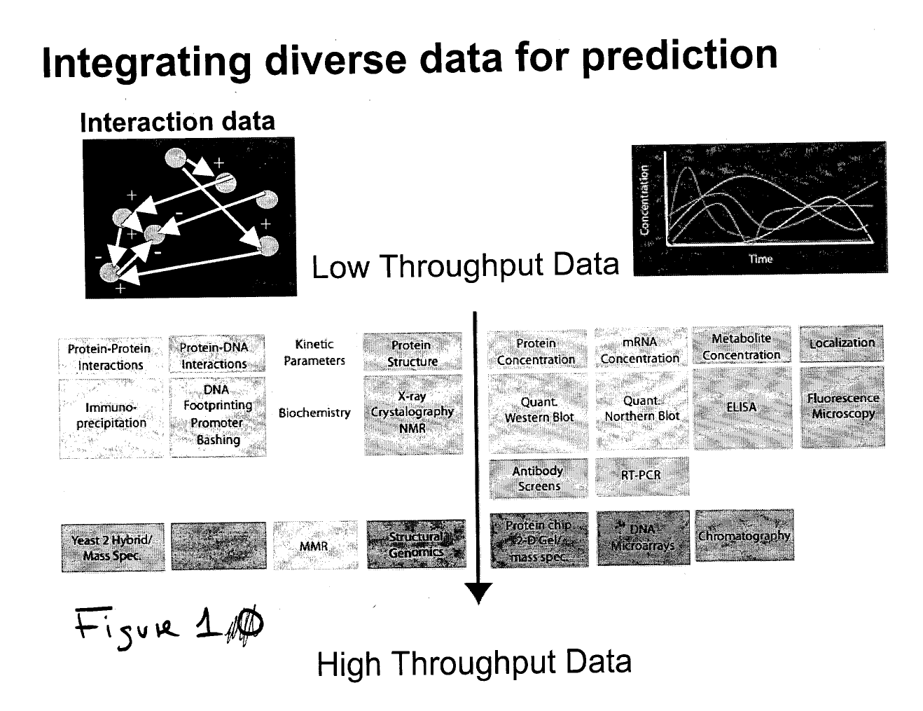

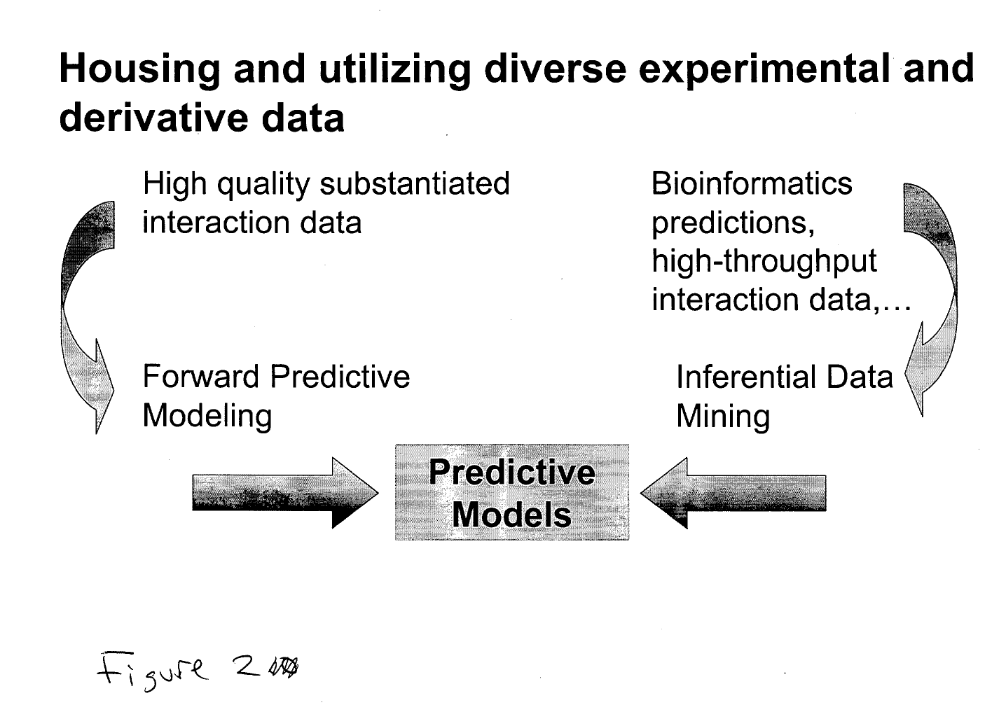

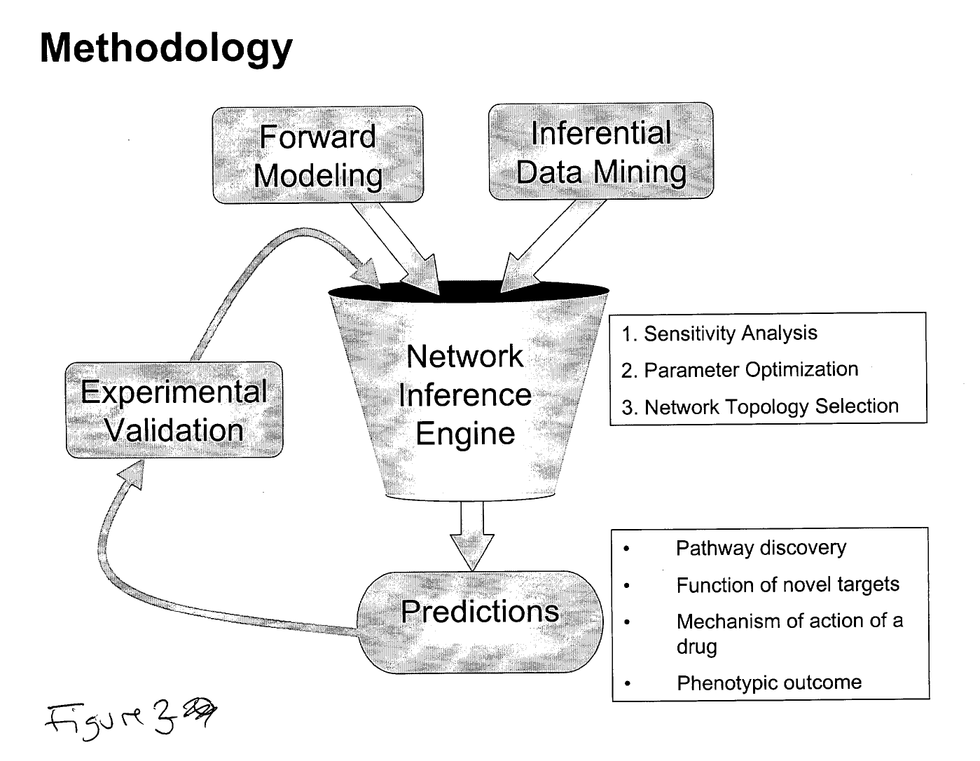

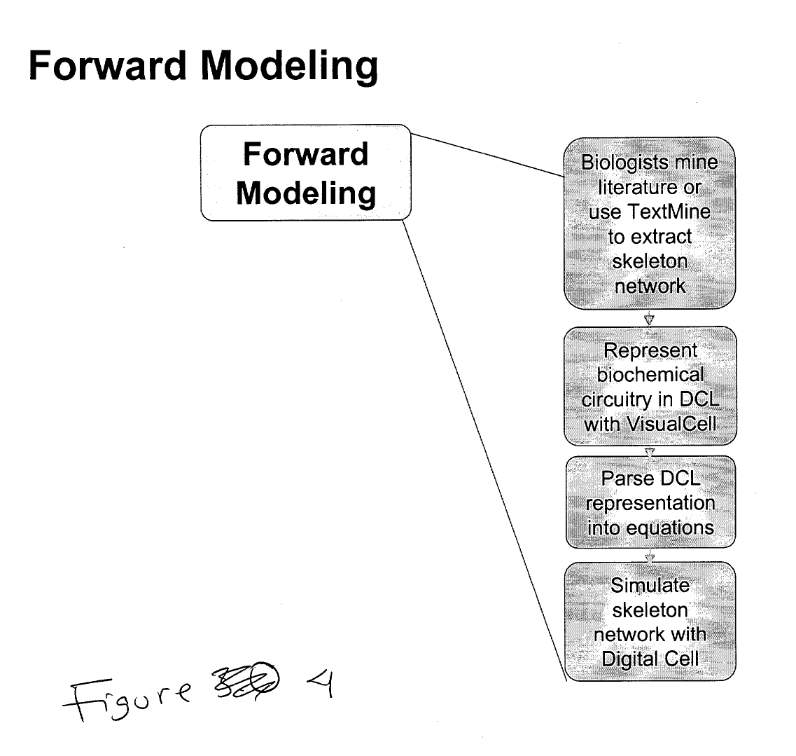

Another exemplary aspect of the invention is the incorporation of data on many levels to account for missing information and thus create a more accurate and predictive model of a biological system. This methodology divides data into three types. The first is interaction data that describes the detailed circuitry underlying the biological system. Included in this data type are protein-protein interactions, protein-DNA interactions, and interaction of genes and proteins with metabolites. This data is used to create a core mechanistic model of the biological system. The second is data that measures the response of the biological system to various perturbations or biological conditions. Perturbations can be in the form of, but not limited to, gene knockouts, addition of a chemical compound or drug, addition of growth factors or cytokines, and any combination of the above. The measured response can be on the phenotypic level and the level of gene, protein, metabolite expression and or localization. The third type of data is derivative data such as DNA and protein sequence from a single or multiple organisms. The second and third type of data is used to both constrain parameters in the model and to infer additional interactions that have yet to be elucidated or discovered (see FIG. 1).

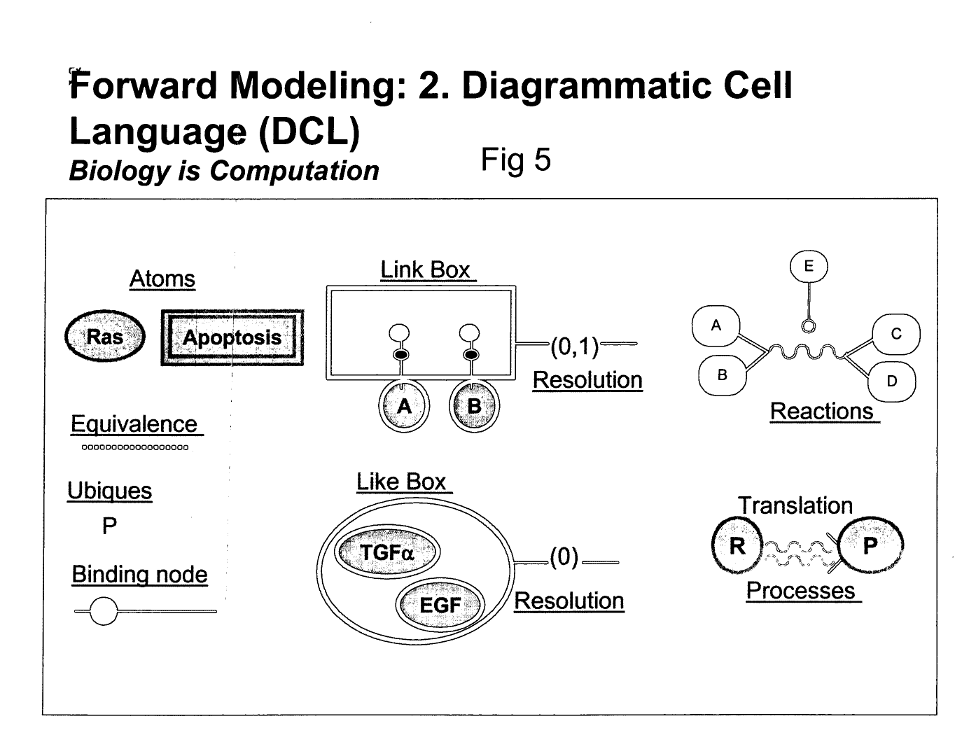

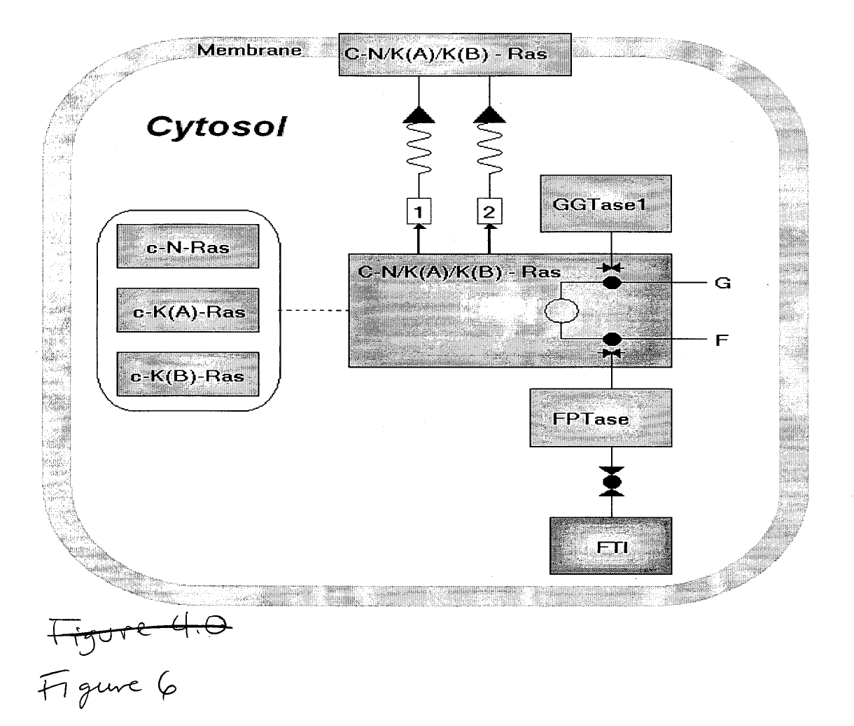

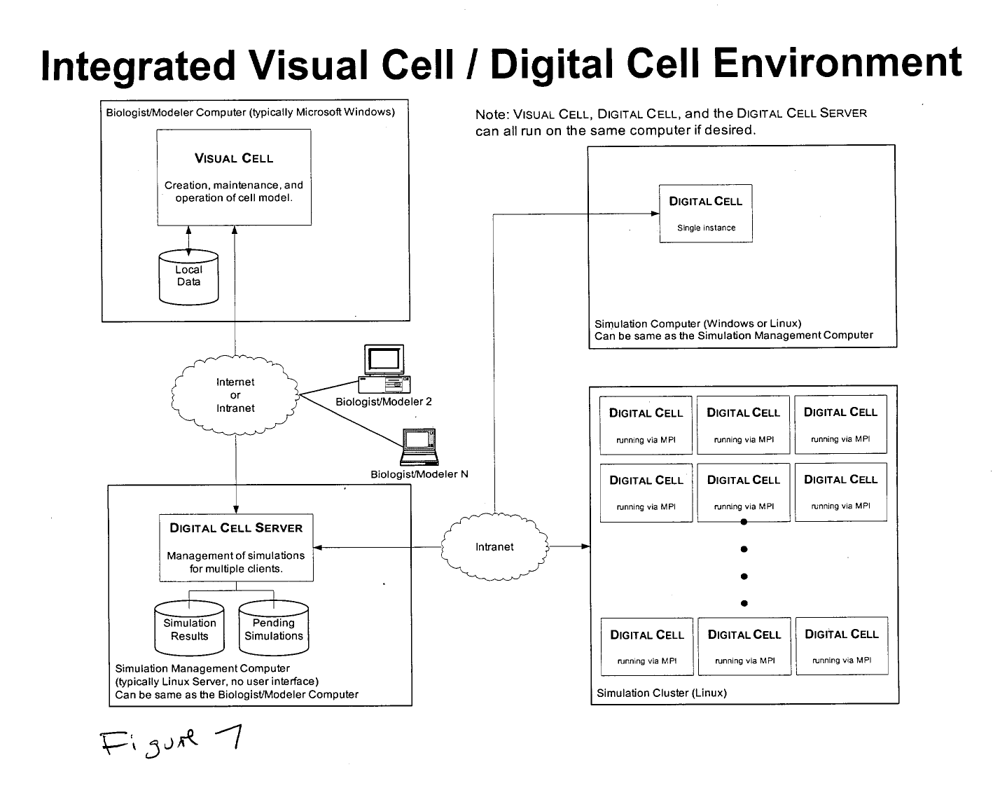

Thus, yet another exemplary aspect of the invention is creation of a framework and methodology for housing and utilizing diverse data types, giving the data the appropriate probabilistic rating, and then using it in the appropriate manner to enhance the predictive power of the model by accounting for all of the possible missing information in the model. This includes kinetic rate constants, concentrations of cellular constituents, and new genes and proteins that have not been discovered or are poorly characterized, but are necessary for describing the behavior of the biological system. High quality substantiated interaction data is incorporated into the core mechanistic simulation. All other data is incorporated into the reverse inferential data-mining approach of the methodology. The two approaches are then combined to create predictive large-scale data driven models of the cell, organism, or biological system of interest (see FIG. 2). The general framework by which these approaches combine is described by the methodology called Computational Hypothesis Testing. This methodology takes as input the core mechanistic simulation built from known biological interaction data and a set of hypothetical reactions gleamed from bioinformatics and data-mining tools and experimental assays (usually experimental methods that have give hints at possible interactions, but still need further substantiation such as a yeast-two hybrid assay). The out put is a population of network models that is a better predictor of the data and the biological system as a whole.

The general techniques comprising the creation of predictive data-driven models of a biological system described in this invention are:

a. Creating a core simulation (scaleable to any size) from known biological information;

b. Collecting quantitative and qualitative experimental data to constrain the simulation

c. Creating a Probable Reactions database by:

i. Analyzing derivative data and experimental via data-mining tools to infer missing interactions

ii. Normalization of confidence levels for a wide variety of data-mining algorithms for extraction of a database of probable interactions

iii. Incorporate other interaction data arising from experimental methods

d. Integrating the core skeletal simulation, the database of probable reactions, and static and dynamical time course measurements to generate an ensemble of biological network structures and their corresponding molecular concentration profiles and phenotypic outcomes that approximate output of the original biological network used for prediction. This technique is called Computational Hypothesis testing and comprises the techniques of:

i. Loop through reactions from the probable reactions database to generate possible network topologies;

1. An important aspect of this is the ability to iterate through a large number of hypothesis in an efficient manner

ii. Weed out networks that have a poor chance of fitting the experimental time course data before applying costly parameter optimization technique;

iii. Constrain the parameter values of the network to fit the experimental dynamic time course measurements using optimization and sensitivity analysis algorithms;

iv. Assign a cost to the network based on several criteria including the cost to fitting the dynamical experimental data

v. Those networks with the minimum cost are deemed highly probable networks; and finally

vi. Billions of hypotheses are weeded out and a subset of hypothetical networks are used to predict biological output. The final output of the methodology is an ensemble of biological networks and their corresponding simulation output that approximate output of the original biological network used for prediction.

e. Validating predictions experimentally and ititeratively refining the model. This technique also includes algorithms that can:

i. Determine which components to measure to discern between various models and model predictions outputted

7.2 Creation of the Core Simulation

This technique is otherwise known as the forward modeling approach. Here one generally begins with knowledge of the components and their interactions in the biological system, and uses it to create a mechanistic mathematical and dynamical simulation of how the cellular constituents work in concert under a number of conditions. Previous efforts in this field tended to focus on creating forward models with relatively few components and interactions (cite previous efforts). However, even the smallest known organism the bacterium M. genitalium contains roughly four hundred genes and at least a similar order of magnitude number of proteins, and a Mammalian cell contains over 30,000 genes and possibly at least an order of magnitude more proteins. It is estimated that at least 10% of the genes in a Mammalian cell are active for a given condition. Thus, a model on the level of the entire cells needs to be of the size of thousands if not tens of thousands of genes and proteins. The methodology described here allows for creating a core skeletal simulation that can scale to the genome wide level. In this technique methods are also described that can simplify the models via creating modules from a group of components and their interactions. A complete cell model can be built up from concrete modules and simplifications can be made for each module. One can use the modular model or description as needed to simplify, understand, and predict the underlying dynamics without having to deal with full complexity of the possible thousands of genes and proteins that may be active in the cell.

The techniques involved in creating the core simulation are:

a) Mining the literature to extract skeletal network

b) Representing the biochemical circuitry using a mathematically concise and scaleable modeling language

c) Parsing the representation into a set of equations or computer instructions

d) Estimating initial values for parameters

e) Simulating the skeletal network

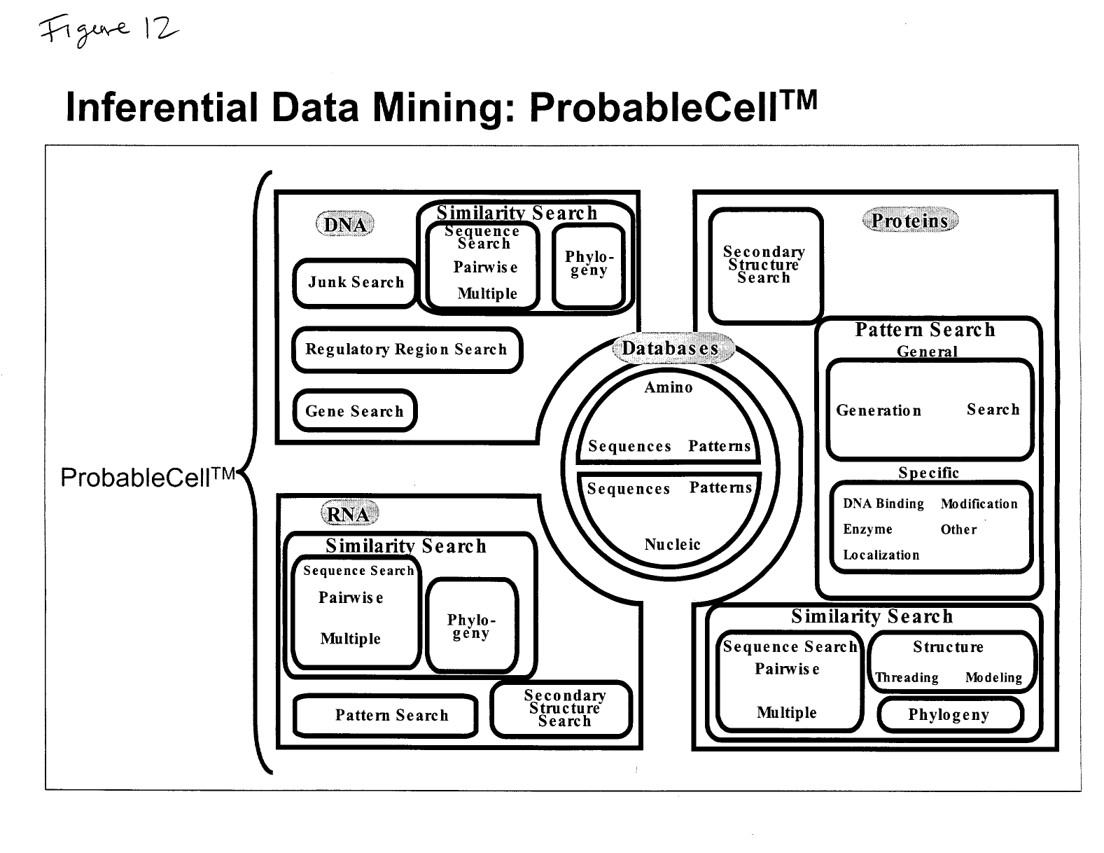

Each technique is described in further detail below. In addition, methods are described for creating a realistic whole cell simulation that includes compartments, modeling discrete events, and connecting the dynamical output of the model to a phenotypic outcome of the cell or biological system. A method for describing the whole cell model in terms of discrete modules allowing for scaling and simplification of the whole cell simulation is described and its extension from the whole cell level to the organism level. FIG. 4 shows the main techniques in this application using the software tools that enable the application.

a) Mining the Literature to Extract Skeletal Network

In this technique a combination of text mining tools and human curators is used to extract the model of the cell or biological system.

As a first technique in this process, text-mining tools can mine PubMed, the full text of journals, and public biological databases to extract the names of genes, proteins, metabolites and other constituents of the cell and then to link these components via the reactions that occurs between them. The relationship extracted by the Text-mining tool can be as simple as the co-occurrence of the gene/protein name in the sentence with another, higher order relationship describing some kind of biological interaction such as a binding between two proteins, or an even higher order relationship describing a more complicated reaction such as protein A promotes the transcription of gene C in the presence of transcription factor D. The text mining tools aid in helping curators find the relevant relationships. Some of the tools that are publicly available can run on massive amounts of text and extract the relevant biology and store it ahead of time in a repository. When the human curator goes in to further define the relationships to create the model, they will already find a rough network in storage. This is an important technique to speeding up the time it takes to curate the biological literature. At some point into the future, text-mining tools may become sophisticated enough that it would abrogate the need for human curation.

The second technique then proceeds to the human curators. They can search for relationships between the genes and proteins directly or use the output of the text-mining tool. Here one is interested in further defining the relationships between components. Then in establishing the validity of the interaction. Many biological interactions in the literature are either ill defined or contradictory. For example, one can look for interaction data on what are the particular binding components of protein A. In one article they might list B and C and the other just B and claim that C does not bind. A system and method is needed to rate the interactions found in the biological literature and determine the validity of what is being stated. Should the curator create a model where protein A binds to both B and C or only B leaving C not bound? To address this, a methodology has been developed for appropriately rating biological knowledge and dealing with contradictions and inconsistencies.

This methodology consists of rating each biological interaction found in the body of biological literature based on the types of experiments done to discover the interaction. The rating system, would for example, evaluate the experiments used to show that protein A binds to B and C versus the experiment used to show that protein A binds to B and definitely not C. The evaluation would consist of categorizing the experiment for:

1) Experiments done in vivo with endogenous protein

2) Experiments utilizing stable transfection

3) Experiments utilizing transient transfection or viral transduction

4) All bioinformatics inferences, in vitro experiments, and high-throuput high error experimental assays such as yeast two-hybrid

The experiments are rated as high, medium high, medium, or weak respectively or via any other appropriate degradation of ratings. All reactions rated as “high” make it into the core skeletal simulation. All other reactions are rated according to the above criteria and put into the probable reactions repository to be used in the Computational Hypothesis Testing framework described below. The Computational Hypothesis Testing framework would still take into account the relative rating of a medium rated interaction versus a weakly rated interaction. The key point to this technique is to identify a rating system for dealing with contradictions and inconsistencies in the biological knowledge based on evaluating the actual experiments, then to create a core simulation that takes into account the most highly rated link and treats the rest in a probabilistic manner based on those links leading to a better fit the experimental data.

b) Representing the Biochemical Circuitry Using a Mathematically Concise and Scaleable Modeling Language

Once the components of the biological system to be modeled have been defined and the interactions between them elucidated from the body of biological knowledge (literature, databases, or even direct experimental measurements), then one needs to assemble this knowledge into something that represents the connectivity between the components: a circuit diagram of the gene and proteins underlying the cell or biological system. One method for doing this is to devise a diagrammatic representation that is concise enough to be translated into a mathematical framework and or a set of computer instructions, but scaleable such that one can use it to represent the complete wiring diagram of the cell. An example of such a language is the Diagrammatic Cell language described in the patent “Cell Language” cited above. The Diagrammatic Cell Language has a diagrammatic component that allows for visual representation of the connectivity of the cell or biological system and an underlying mathematical and computational component that allows for automatic translation to computer code. Using this language, GNS has been able to construct a whole cell mammalian model with over 500 genes and proteins and 2000 states/chemical species and reactions (a state is define as a component in a modified form such as a protein being phosophoryalted, or bound to another protein, or both bound and phosphoryalted).

Diagrammatic Cell Language (DCL)—Knowledge Representation

Biology is Computation The Diagrammatic Cell Language

Biology is not just chemistry; biology is chemistry with a function. But function is ill defined. When a protein binds to DNA, it may produce a protein. But is this the function? Perhaps the protein will also block a second molecule from binding. Is this the function? Perhaps the binding will prevent the protein from catalyzing a reaction in some other pathway, far off. It is difficult to disentangle the different functions

from one another. But there is one central dogma that gives an unambiguous meaning to function: the computational interpretation of biological function. The function of the molecule is the algorithm it executes on its surroundings.

The Diagrammatic Cell Language allows a biologist to describe the function of a biological component in a mathematically precise way. The diagrams allow biologists to communicate with modelers, and modelers to communicate with machines, because it describes the structure of the network in a form that is both human and machine-readable. The mathematical equations that describe the biological system may be derived from a graphical representation that is designed to describe the function of molecules that transform one another; the transformed molecules then act to transform other molecules, and on, in a cycle. The processes taken together form a model for the cell.

The Diagrammatic Cell Language is a computationally complete language. The language is dynamic and contains complex objects which may inherit properties from their constituents. This type of inheritance is inspired by object-oriented languages, but it is different in one crucial way: the objects do not lie in a hierarchy. Whenever a number of objects produce a new one by any process, the resulting object inherits all the properties of all the objects, except for those specifically excluded.

The function of the components is what the language seeks to represent, not their sequence or structure, and this is a departure from the focus of classical biology. Although the structure of a protein is difficult to determine from general principles, once the effect of this protein on other proteins is known, the structure is no longer important. Whatever the structure, the function of the protein determines the same graph. To be more precise, it determines one of a number of equivalent graphs. As in any other description of an algorithm, there is some redundancy in the graphical description of the network. In practice, the smallest graphs are very concise.

The Language is designed for mechanistic parsing into a simulation: deterministic, stochastic, or discrete. The result is a state-machine that can reproduce the biology of the cell. The two central concepts are linkboxes and likeboxes. Linkboxes surround a physical combination of different objects, which could be binding sites on a protein, regulatory regions of DNA, or different conformations of a chain. Likeboxes are combinations of objects that act alike, that have a similar function. A sample of the objects that empower the language is shown in FIG. 5.

### ILLUSTRATIVE EXAMPLE

A biologist mines the literature and extracts the following textual information on the reactions that the various isoforms of Ras undergo as a result of interacting with two particular enzymes:

“Farnesyl protein transferase (FPTase) catalyzes the addition of a famesyl group onto C-N-Ras, C-K(A)-Ras, and C-K(B)-Ras. Faresyl transferase inhibitor (FTI) blocks this reaction. Some classes of FTIs work by irreversibly binding to FPTase. Geranylgeranyl transferase 1 (GGTase 1) catalyzes the addition of a geranylgeranyl moiety onto the same group of Ras molecules. After lipid modification, each Ras molecule translocates from the cytosol to the membrane.”

This information can be modeled concisely in DCL by representing the three isoforms of Ras in a like box shown in FIG. 6. Then drawing an equivalence line from that structure to an arbitrary link box called C-N/K(A)/K(B)-Ras, where it can get modified by a Farnesyl group abbreviated as F, and a Gemysal group abbreviated by G. The component FPTase causes the addition of F and the component GGTase1 causes the addition of G. When C-N/K(A)/K(B)-Ras, representing each isoform of Ras singly, is modified and in either the state [1] or the state [2] it undergoes a reaction where it undergoes a translocation from the cytosol to the membrane. The biologists would continue to uncover biological information and map more and more of the circuitry until a complete model is developed. There is not limit to the scale of the model using this tool.

c) Parsing the Representation in to a set of Equations or Computer Instructions

Once a circuit diagram is constructed of the gene and proteins underlying cellular function, one then needs to “parse” or translate the description into a mathematical formalism or a set of instructions readable by a computer. Starting from the sub-pathway shown in FIG. 6, the specific reaction steps can be directly read off as:

1.GGTase1 catalyzes the reaction of c-N-Ras to c-N-Ras_G

2.FPTase catalyzes the reaction of c-N-Ras to c-N-Ras_F

3.c-N-Ras_G transforms to c-N-Ras_G_membranebound

4.c-N-Ras_F transformed to c-N-Ras_G_membranebound

5.GGTase1 catalyzes the reaction of c-K(A)-Ras to c-K(A)-Ras_G

6.FPTase catalyzes the reaction of c-K(A)-Ras to c-K(A)-Ras_F

7.c-K(A)-Ras_G transforms to c-K(A)-Ras G_membranebound

8.c-K(A)-Ras_F transformed to c-K(A)-Ras G_membranebound

9.GGTase1 catalyzes the reaction of c-K(B)-Ras to c-K(B)-Ras_G

10.FPTase catalyzes the reaction of c-K(B)-Ras to c-K(B)-Ras_F

11.c-K(B)-Ras_G transforms to c-K(B)-Ras_G_membranebound

12.c-K(B)-Ras_F transformed to c-K(B)-Ras_G_membranebound

(*Note the compactness of the DCL representation)

These reaction steps can then be correlated with a specific kinetic form such as mass action balanced kinetics or higher order kinetics like Michaelis-Menten given by: [Substrate] k/(Km+[Substrate]), where k and Km are rate constants. For example for the steps 1-4 one can choose the reasonable mass action kinetic forms and stoichiometry of:

| Reaction Step | Kinetic Form | Stoichiometry |
|---|---|---|
| 1. GGTase1 | [GGTase1] [c-N-Ras] kb_GGTase1 c-N-Ras | 1 [c-N-Ras] is removed |
| catalyzes the | [GGTase1:c-N-Ras] ku_GGTase1_c-N-Ras | 1 [c-N-Ras G] icreated |
| reaction of c-N- | [GGTase1:c-N-Ras] kt_c-N-Ras_G | [GGTase1] no change |
| Ras to c-N- | | |
| Ras_G | | |
| 2. FPTase | [FPTase] [c-N-Ras] kb_GGTase1_c-N-Ras | 1 [c-N-Ras] is removed |
| catalyzes the | [FPTase:c-N-Ras] ku_GGTase1_c-N-Ras | 1 [c-N-Ras_F] created |
| reaction of c-N- | [FPTase:c-N-Ras] kt_c-N-Ras_G | [FPTase1] no change |
| Ras to c-N- | | |
| Ras_F | | |
| 3. c-N-Ras_G | [c-N-Ras_G] kt_c-N-Ras_G_membrane | 1 [c-N-Ras_G] is removed |
| transforms to c- | | 1 [c-N-Ras_G_membrane] |
| N- | | created |
| Ras_G_membranebound | | |
| 4. c-N-Ras_F | [c-N-Ras_F] kt_c-N-Ras_F_membrane | 1 [c-N-Ras_F] is removed |
| transformed to c- | | 1 [c-N-Ras_F_membrane] |
| N- | | created |
| Ras_G_membranebound | | |

Where the kb_, ku_, kt_{—} represent the rate constants for those reactions.

The basic methodology of going from a compact and concise diagrammatic representation such as the one shown in FIG. 6 is: (1) state the reaction steps encoded in the diagram, and then (2) assign a kinetic form to each reaction step. An algorithm can then be programmed that compiles the various reaction steps into a set of differential equations, their stochastic counter parts, or a hybrid method described below. Several software packages exist that can take as input reaction steps tied to kinetic forms and solve the system as a deterministic set of differential equations, stochastic equations, or hybrid methods. An exemplary embodiment of such a tool is the DigitalCell simulation package that contains this functionality.

As an example, the differential equation for c-N-Ras is:

d[c-N-Ras]/dt=−[GGTase1] [c-N-Ras] kb_GGTase1_c-N-Ras+[GGTase1:c-N-Ras] ku_GGTase1_c-N-Ras-[FPTase] [c-N-Ras] kb_GGTase1_c-N-Ras+[FPTase:c-N-Ras] ku_GGTase1_c-N-Ras

An important aspect to being able to efficiently model biological systems both in terms of speed and scalability is to create a software environment that allows for easy implementation of the model, both in representing it diagrammatically and in translating the representation to mathematical code automatically. Specific embodiments of the tools used to perform this are the VisualCell graphical environment and the DigitalCell simulation environment described below.

Unified Modeling and Simulation Environment-From DCL to VisualCell to DigitalCell

Two main software tools that enable the ease of using DCL to represent pathways and the simulation of those pathways are the VisualCell and DigitalCell, respectively. VisualCell is a highly graphical application which enables visual editing of cellular signaling networks and other biochemical networks. DigitalCell is a dynamical simulation engine currently supporting numerical integration of ordinary differential equations and stochastic time evolution.

VisualCell is currently used to create, annotate and communicate the network. The networks are typically constructed by biologists specializing in a particular pathway and then passed either digitally or via printouts to a mathematical modeler who uses the diagram and the annotations to create lists of reactions that s/he types into a specific text file format. This file format is input to and parsed by DigitalCell, which constructs differential or stochastic equations, and solves them via a variety of user-selected methods. The output from DigitalCell is in the form of a time course for each chemical in the network that the user specified s/he wanted to see.

While this method has been successful so far, it is inefficient and will not scale to very large networks. It is desirable to eliminate the need for the intermediate text files, and to directly integrate the visualization of the network, the numerical simulation of the network, and the simulation results. An environment in which a biologist can construct and manipulate a dynamic, graphical depiction of a signaling or metabolic network will enable much larger systems to be modeled than are currently possible. A schematic of the unified environment is shown in FIG. 7 and a screen shot of the interface in FIG. 8.

The environment allows for the software-assisted, conversion of the network diagram into the lists of reactions, chemicals, and parameters from which differential or stochastic equations are generated by DigitalCell. Since the simulation of even a simple network can require transcribing thousands of reactions, this step is particularly important. The environment also enables the seamless communication between a graphical application that typically runs on a personal computer with a numerical application that typically runs on a computer cluster. Some optimizations can take days to run, so the interaction has to include asynchronous as well as synchronous management of simulation runs.

d) Estimating Initial Values for Parameters

Before solving the systems of equations underling the circuitry of the cell to produce simulation output, initial values for parameters in the model must be set. These parameter values include rates for transcriptional activation, translation of proteins, binding between proteins, binding rates between genes and proteins, translocation rates of components between various cellular compartment, . . . etc. The parameters also include initial concentrations of molecules in the cell and any other parameter values that the simulation depends on. One source for getting parameter values is the biological literature and databases. This can give the exact values needed in the simulation of give a reasonable starting value for the optimization algorithms to search around. The second is via direct experimental measurements. Another way is to estimate the initial value from known values of genes and proteins with similar functionality.

For example, one can use the initial value of a particular ligand binding to a particular receptor that is representative of other ligand receptor binding rates. For Mammalian cells one finds binding and unbinding rates that range from x to y. Similarly for concentration levels one find that receptor levels range from a few thousand molecules per cell to tens of thousands. This can be used to give a reasonable starting value for concentrations or a range to restrict the parameter over. Another method for estimating initial values is to use data on the timing of the reaction or reaction steps in a pathway. Does the signal take a few seconds to get propagated or a few hours. The rates of such processes would then be on the order of 1/seconds to 1/(few hours) respectively. It is actually not required that one give reasonable starting estimates for rate constants and concentrations. In doing so, though, the optimization algorithms described in section can more quickly and efficiently find the minimum as it gives it reasonable starting place to search around and can be used to restrict the region over which the optimization searches over.

e) Simulating the Skeletal Network

Once the reaction steps of the model have been defined and the parameter values set, one proceeds with solving the equations underlying the model to create a dynamical simulation. The possible ways that one can compile the reaction steps is to use differential equation framework, a stochastic framework, and hybrid methods that treat some components deterministically and others stochastically. Also, it is desired to combine algebraic statements and or explicit computer instructions (such as a switch that gives the one value of the component before a certain time or event, and another value after) with the above mentioned simulation frameworks. Time delays are also included to approximate the solution to a component undergoing many reaction steps before the final transformation. Note that often time when a simulation is started the system is not at equilibrium. The equilibrium solution must first be found before taking the system and perturbing to observe its behavior forward in time.

DigitalCell—Numerics

DigitalCell is an exemplary embodiment of a simulation/optimization engine. It takes as input the chemicals (species), parameters and reactions describing a bio-chemical network. DigitalCell is written in C++ and makes use of object oriented programming techniques such as polymorphism and hence is highly extensible. For example, all reactions are derived from a Reaction base class. Each derived reaction class must provide methods for calculating the rate and the contribution to the Jacobian matrix. This structure makes adding new reactions to the code very simple.

i. Numerical Integration of Multi-scale Systems

a. Deterministic and Stochastic Time Evolution, Including Stiff Systems ODE and Stochastic Solvers

DigitalCell has both deterministic and stochastic integrators. Both types require a set of reactions as input. The deterministic integrators use the reactions to construct a system of ordinary differential equations (ODEs). These systems of ODEs are usually stiff. Deterministic ODE Solvers. DigitalCell has two choices for solving systems of ODEs. The first uses the CVODE solver which is part of SUNDIALS (Suite of Nonlinear and Differential/Algebraic equation Solvers) developed in the Center for Applied Scientific Computing (CASC), at the Lawrence Livermore National Laboratory [67]. CVODE can solve both still_(using Backward Di_erentiation Formula (BDF) methods) and non-sti_(using Adams-Moulton methods) initial value problems (IVPs). For sti_systems, the linear systems that arise can be solved by direct (dense or band) solvers or by a preconditioned iterative method (GMRES) for which the user can supply a preconditioner. CVODE can also be used (in a variant called CVODES) to compute sensitivities.

The other deterministic ODE solver in DigitalCell uses routines from the NAG Fortran Library. The NAG library provides both sti_(using BDF methods) and non-sti_routines. The stiff solvers can use the direct sparse solvers in the NAG library to solve the linear systems.

b. Stochastic Solvers:

The stochastic method provided by DigitalCell is the Next Reaction Method developed by Gibson and Bruck [17] which is a modification of Gillespie's First Reaction Method. At each iteration of Gillespie's method, the propensity of each reaction is computed, then for each reaction a “putative” time is generated. The putative time is the time at which that reaction would occur if no other reaction occurred first. Finally the reaction with the smallest time is executed and all the propensities are recalculated. Gibson's method makes this process more efficient by using a dependency graph to determine which propensities need to be recalculated after a given reaction occurs. Thus each iteration becomes much cheaper since it is not necessary to recalculate all the propensities. In addition, it uses an indexed priority queue to keep track of the times at which each reaction will occur, so it is not necessary to search all the times to find the smallest. Since there is nothing in each iteration that is proportional to the number of reactions, this method scales well to very large networks.

c. Hybrid dynamics

The bacteria model has two formulas for the volume of the cell—one which depends on the chemical levels and their densities, and the other which depends on the geometry of the cell (width, length, number of septa and their lengths). There are a number of discrete events in the model as well. For example, when the cell divides, the surface area and volume are halved, the number of septa decreases by one, what was the secondary septum becomes the primary septum, etc. Thus, the bacteria model is an example of the type of hybrid differential Algebraic Equation (DAE) system that is handled by the DigitalCell.

d. Equilibrium solvers

The DigitalCell has equilbrium solvers that utilize two methods. One is Newton(s) method which tries to solve the system of equations of:

d[A]/dt=0;

The other integrates out to infinity (where infinity is a very large time step) to find the starting equilibrium solution. The two methods also work in combination using the infinity solution as a starting point for the newton's solver.

Additional Aspects to Creating a Realistic Whole Cell Simulation Extensible to the Tissue, Organ, and Organism Level

In addition to identifying the cellular constituents to model such as the genes, proteins, and metabolite one needs to identify spatial aspects and input their static or time dependent behavior. For a whole cell simulation cellular compartments need to identified such as the cytosl, nucleus, mitochondria, and endosome. Components can be localized in these compartments or can translocate between various compartment. In addition, (see the exemplary examples of the E. coli can cancer models), the time evolution of continuous components needs to be tied to discrete stochastic events. Such as cell volume changes, DNA replication, cell division, and mitochondrial disruption. These events tie the detailed biochemical processes modeled to physiological processes that must be modeled in order to predict both expression of genes, proteins, and metabolites their localization and the final phenotypic response of the cell on the physiological level.

The biochemical pathways within the whole cell model can be divided into discrete modules, where a module is defined as a group of interacting components with some input and output signal going into and out of the module. The modules can be modeled and optimized separately as a strategy, then connected up. Components can be shared with more than one module (as is often the case with the abundant cross talk in biological systems). Modules are then hooked up together and modeled as a complete unit. Additional components can be added to each module and scaled accordingly. In this way, the entire cell can be thought of as a module. From this populations of cell modules can be modeled and organized spatially to form models of tissues, organs, and eventually the entire organism level.

1.2 Quantitative and Qualitative Experimental Data Collection

In order to train the model to infer kinetic parameters and missing interactions and components, a data set can be generated that reflects the dynamic behavior of the biological system. Several methods exist for measuring components on the RNA, protein, and metabolite level. For protein measurements one can utilize anything from Mass Spectrometry methods to protein chips. For RNA one can use real time—PCR for low through put measurements and microarray chips for high-throughput measurements. Methods that utilize high-throughput means are desired as one can generate enough data on as many constituents as possible to fit a large-scale model.

Data can also be used on the physiological level as long as there is a way to correlate the physiological rezones to a component in the simulation. For example, caspase activity can be indicative of cell death via apoptosis. For typical mamalina cells dynamical data can be generated by:

Treating with desired perturbation

Generate enough time

points to capture

dynamical behavior

Time range can vary over 12-48 hour

per treatment

15-30 time points/exp (15-30 DNA chips)

Repeats of each measurement to obtain error bars

FIG. 9 contains a time course measurement of Caco-2 cells under EGF stimulation that can be used to constrain a model.

1.4 Probable Reactions Database

As was mentioned, many of the functionality and interactions between components (e.g. genes, proteins, metabolites) in the cell are not known. How does one then account for the unknown components and interactions and then realistically incorporate this information into a dynamical simulation designed to predict the behavior of the cell? One method for extracting additional information on the circuitry of the biological system is to use bioinformatics tools that identify patterns and correlations within the chemical sequence (e.g. DNA sequence and protein sequence of the organism). The other is to analyze data emanating from high-throughput experimental methods such as measurements of mRNA levels using microarrays. The analysis of such data can be combined with sequence analysis to generate additional and more robust predictions. Precedence for using such tools to generate bioinformatics predictions exists in the literature (for an example, see). What has not been dealt with is methods for normalize predictions emanating from different data-mining algorithms and methods to generate more robust predictions. In addition, ways to then combine predictions from sequence analysis, high-throughput measurements, combinations of the two, and other sources in a hypothesis for a complete reaction. The various hypothetical reactions generated are all stored in a central repository called the probable reactions database. Once this is established, the various hypothetical predictions inferred and stored in the probable reactions database can be tested in a more rigorous computational framework described in the Computational Hypothesis sub-section. FIG. 10 shows the process used to analyze derivative and experimental data and the repository for the data called probable reactions database. FIG. 11 shows specific tools for analysis of such data described in the patent “Bioinformatic data mining system”.

1.4.1 Data-Mining Inferences from Derivative and Experimental Data

Two types of data lend themselves to analysis for deriving predictions on unknown interactions in a biological system. The first is derivative data such as gene sequence and protein sequence of an organism. The second is experimental data on the expression and localization of constituents in the cell. For example, this includes protein expression measurements using mass spectrometry methods and mRNA expression measurement using microarrays. Analysis of such data types can yield information on possible transcription factor motifs, transcription factor binding sites, possible transcription factors that bind to these sites, protein binding motifs, protein-protein interactions, and predictions on protein modifications, to name a few. Once a prediction is made on, for example, whether a binding motif for a transcription exists and the possible transcription factors that bind to it, this can then be used as a possible hypothetical reaction to further investigate. This can be done by direct experimental measurement or using the Computational Hypothesis testing framework described below which essentially tests the possible hypothetical reaction within the context of the core simulation and compares it to experimental data. Reactions that improve the cost to fitting the data are deemed highly probable and would warrant further investigation. Inclusion of such reactions also improves the predictive power of the core simulation, which may be missing many relevant biological interactions.

To improve the inferences made by the bioinformatics tools the methodology integrates all non-redundant bioinformatics algorithms. Thus multiple tools that use inherently different methods are used to generate inferences. An inference where there is agreement using more than one tool would have a higher probability of having biological relevance. These are the inferences that are usually retained and passed on to the probable reactions database for testing within the Computational Hypothesis framework. The bioinformatics algorithms can mine sequence data alone or a combination of sequence and expression data.

Specific Embodiment in the ProbableCell

The ProbableCell is a specific embodiment of a platform that integrates all non-redundant bioinformatics algorithsms shown in FIG. 12. The ProbableCell is an object database that is a repository for all the information that is obtained by bioinformatics or high-throughput measurements. The basic architecture is an object structure, with messages corresponding to bioinformatics algorithms that either lead to the construction of a new type of object (e.g. gene prediction algorithms returning a list of predicted gene 34 objects in a portion of a microbial chromosome), or return some property of an object (e.g. a helix-turnhelix predicting algorithm returning a probability of the occurrence of such a structure in a protein as a response when applied to that protein object). This architecture is modular, easily allows the addition of new algorithms, and is able to handle the many-to-many relationships that arise in these predictions.

To date, the embodiment implemented has a set of algorithms in the object structure indicated above; constructed an object database schema for storing these objects in an object database, and tested the performance of object databases as implemented by Objectivity; constructed a preliminary graphical user interface for the object database, which allows users to click on an object and apply different bioinformatics methods to that object, either to generate other objects in the database, or to derive some property of the object being considered; and constructed a unified preliminary format for presenting all known bioinformatics results for a given protein.

Protein sequences carry information about the function of the protein. Correlations between two or more amino acids in different relative positions along the chain relate, statistically, to specific biological properties. Although the space of sequence-function correlations will always house mysteries, many methods are available to search a query sequence for these signals. Some tools match sequences against a database of patterns that fit some general format (e.g. the PROSITE database). Such tools are capable of recognizing different patterns within the same sequence. Other tools rely on algorithms tailored for identifying one specific pattern, e.g. a helix-turn-helix motif. Another tool is capable of combining the results of multiple signal sequence identification algorithms into a prediction of subcellular localization. At present the embodiment has 15 such “motif search” tools automated. These include database search tools for the PROSITE, PRINTS, BLOCKS, and PFAM databases (cite references for the websites of those tools).

1.4.2 Normalization of Data-Mining Inferences

The methodology described in this patent is designed to generate, and to invalidate hypothetical interactions, in biological systems. There are an extremely large number of hypothetical molecules with putative functions that are the building blocks of models of biomolecular function. As such, the number of hypotheses that may be generated is super exponential in the number of components, and must be prioritized for systematic investigation. This prioritization starts with consistent and commensurate confidence levels for bioinformatics predictions. Most bioinformatics algorithms provide confidence levels for predictions. The problem that must be treated carefully when integrating results from different algorithms is to convert all confidence levels to a common standard, e.g., to probabilities, and then use the calculus of probabilities to compute when different algorithms predict the same information. If the algorithms are fundamentally different computations, e.g. taxonomy based on secondary structure vs. tertiary structure calculations, predictions that are in agreement should be ascribed a lower probability of consistency with an appropriate null hypothesis, as opposed to predictions that disagree. The calculus of probabilities will allow this provided that the probabilities are computed in the same framework for with prediction.

Thus, the aim of this part of the project will be to

separate algorithms carefully into algorithmically distinct groups, and calculate probabilities for each algorithm against a consistent null hypothesis;

formulate appropriate null hypotheses for each type of prediction

evaluate consistency with the null hypothesis for any given prediction.

Two possible ways of calculating such probabilities that will be investigated are: (a) Using statistical distributions of the algorithm's confidence levels to convert the confidence levels into probabilities; and (b) For sequence based algorithms, to use quasi-random sequences, with correlations similar to the actual biological sequences up to some base or residue separation, as the null sequence' to ascertain the likelihood of the predicted structure in the quasi-random sequence.

1.4.3 Incorporation of all Interaction Data into the Database of Probable Reactions

The restrictions obtained on network architecture from the pattern discovery algorithms, and taking into account the information extracted from the literature still leave too many possible interactions in the biochemical network governing the behavior of the microbe that are undetermined in their detailed form. In most regulatory systems not only is a protein involved in binding DNA to affect transcription, but that protein is activated or deactivated in response to some signal, typically a small molecule effectors. The identities of those effectors are usually unknown. Bioinformatics predicts some properties of biological molecules, based either on ab initio computational algorithms, or on algorithms that search for similarities with experimentally obtained information about other molecules. These bioinformatics predictions are usually not in themselves the basis of a hypothesis that could be directly tested in a computational model of biomolecular dynamics. For example, a transcription factor binding site algorithm might predict a putative binding site, but usually will not provide information on the cognate transcription factor(s). On the other hand, a protein secondary structure prediction algorithm may predict a helix-turn-helix structure, but will not have any information on the appropriate binding site for this putative transcription factor. A major effort in bioinformatics will be devoted to evaluating different rule based approaches to derive hypothetical interactions based on integrating bioinformatics predictions, text-mining and the analysis of high-throughput datasets. We refer to this set of hypothetical interactions as the Probable Reaction Database in the following. Several important aspects of this problem are the key focus of this research:

Evaluation of possible combinations of predictions that amount to a hypothesis for a complete reaction.

‘Interactions’ between items in the Probable Hypotheses Database, arising from competing or synergistic hypotheses.

Presentation of these results in a manner suited for modelers to consider incorporating into a model.

Use of these results in automated Computational Hypothesis Testing on a large scale.

The Probable Cell may be thought of as a repository of data regarding putative biological molecules. This part of the project will aim at producing putative biological interactions from this data, in other words, attempt an integration of molecule-centric data into interaction-centric information. Even when some of the data, for example, data arising from automated text-mining, is actually in the form of suggested interactions between molecules, it still requires analysis before it can be treated as a hypothesis suitable for incorporation into a dynamical model. As mentioned in, putative transcription factors and their cognate binding sites are computed with some element of uncertainty, even with phylogenetic information taken into account. This is a source of simple hypotheses for interactions.

1.5 Computational Hypothesis Testing

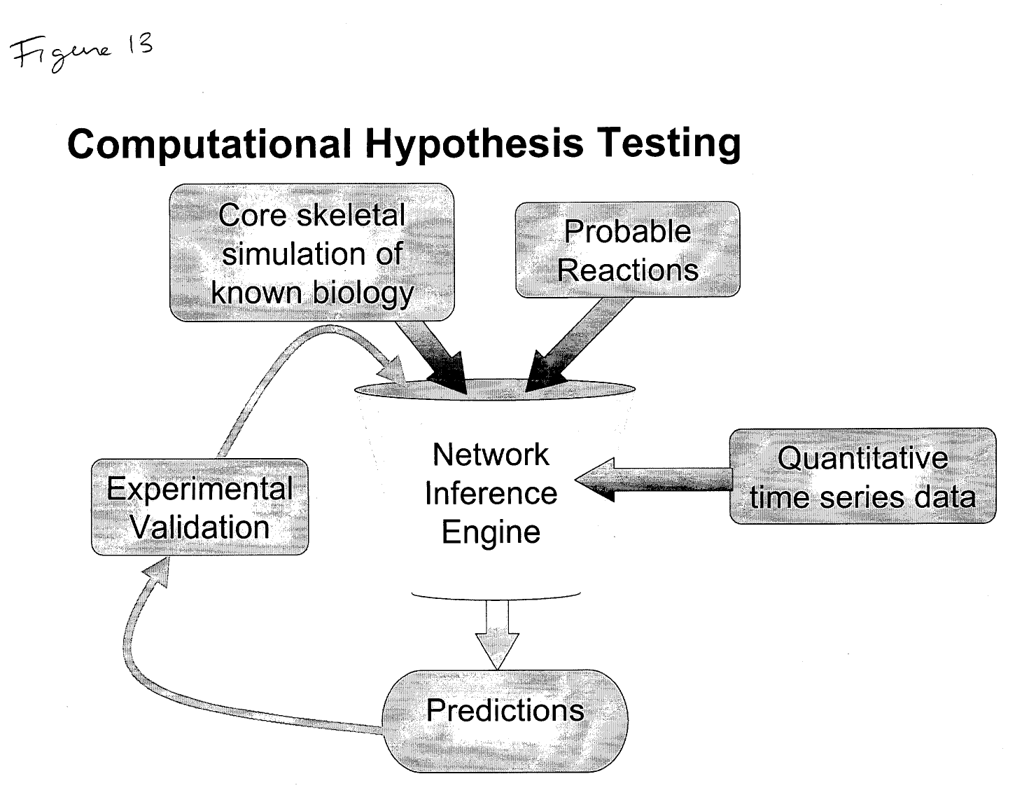

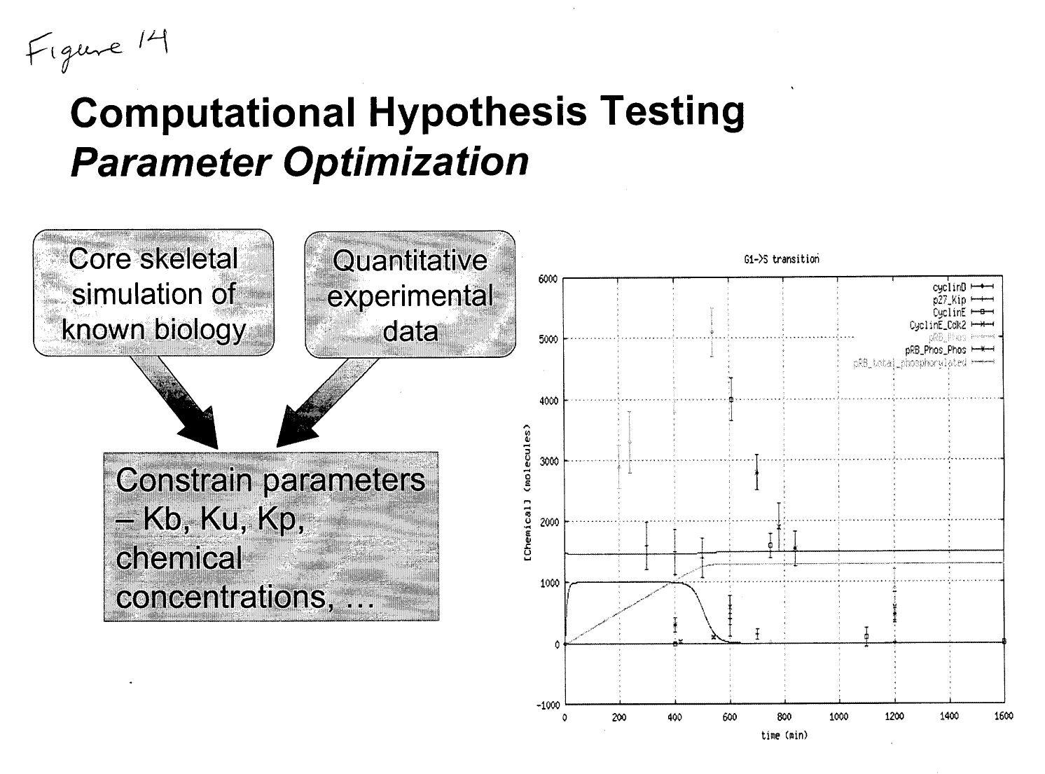

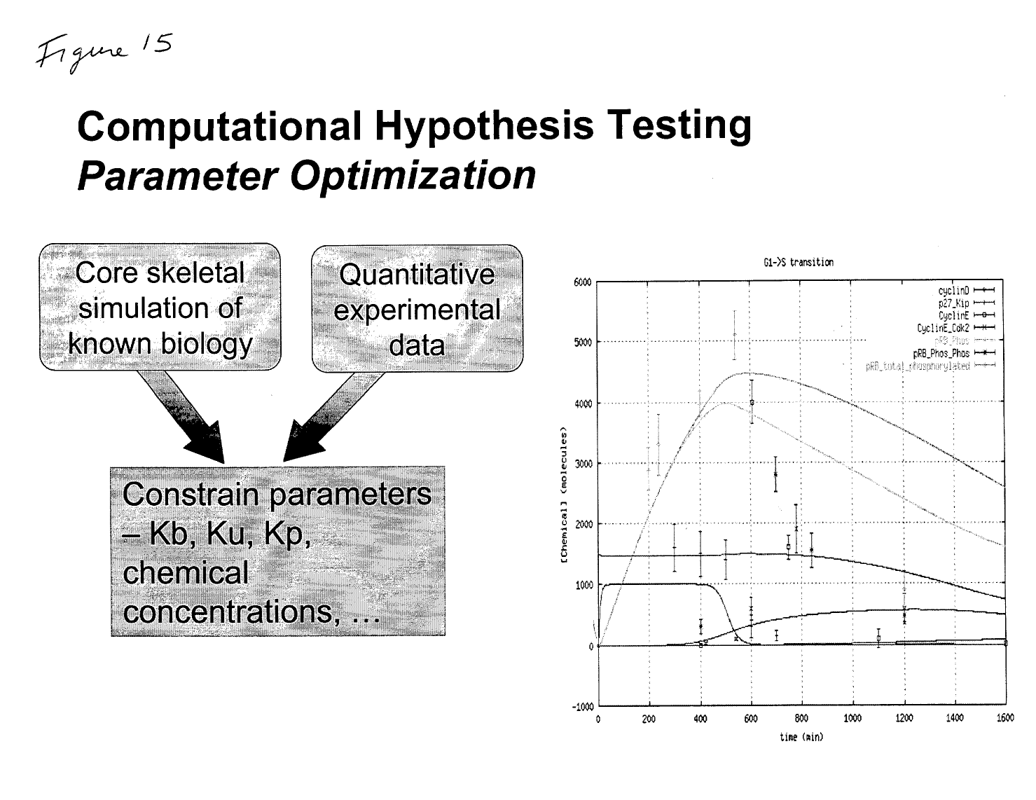

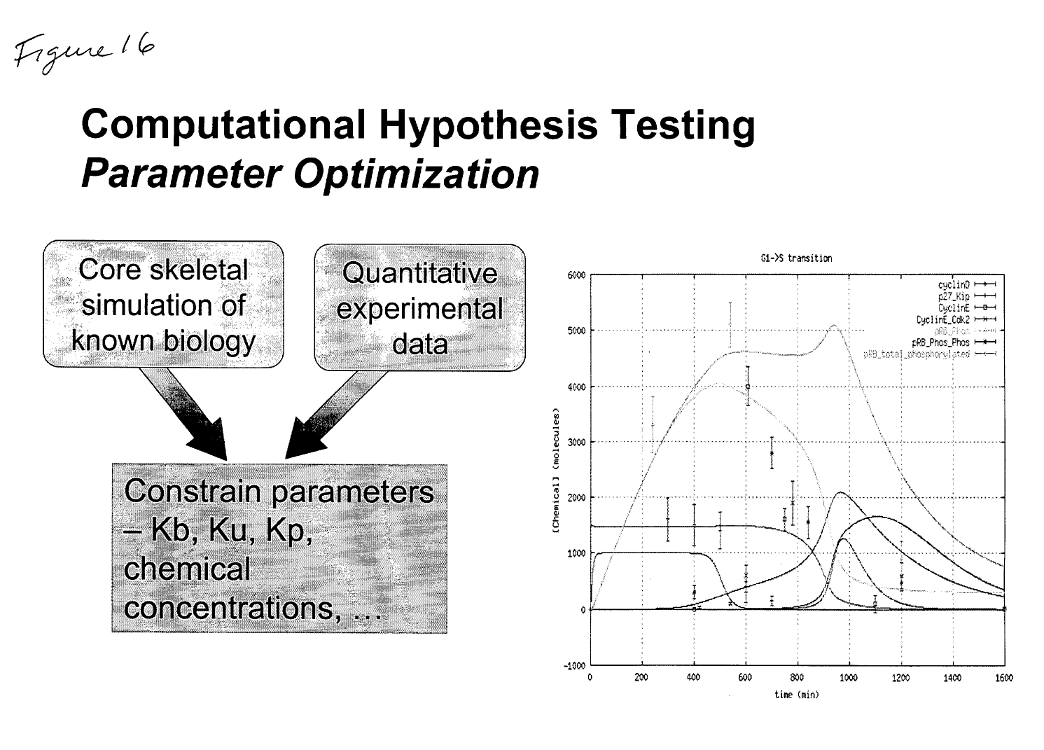

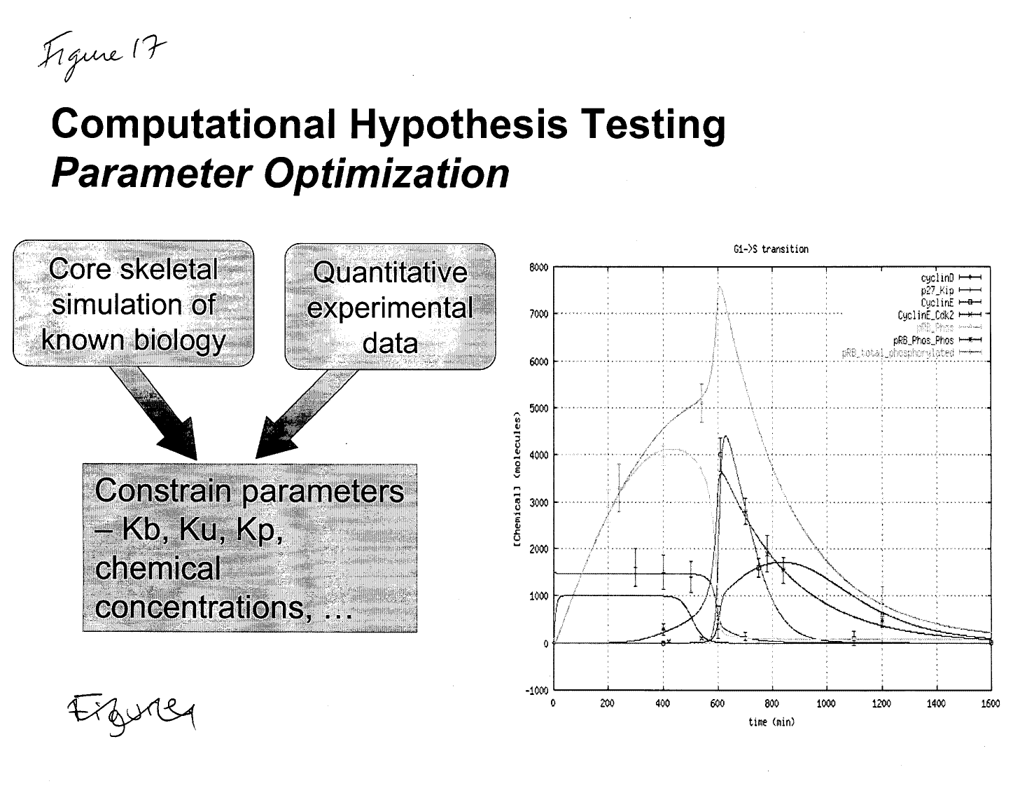

Each component described so far comes together via the Computational Hypothesis Testing methodology shown in FIG. 13. The main inputs into this methodology is the core simulation of known biology, the database of probable reaction, and dynamical time course measurements. These serve as inputs into what is labeled the Network Inference Engine in FIG. 13. The Network Inference Engine contains the algorithms that will evaluate the core simulation for being able to fit or match the experimental time series data and then successively iterating through the reactions in the probable reactions database to find a better network topology or topologies that fit or match the experimental data.

The general techniques performed by the Computational Hypothesis Testing framework are: (a) loop through reactions from the probable reactions database to generate possible network topologies; an important aspect of this is the ability to iterate through a large number of hypothesis in an efficient manner; (b) weed out networks that have a poor chance of fitting the experimental time course data before applying costly parameter optimization technique; (c) constrain the parameter values of the network to fit the experimental dynamic time course measurements using optimization and sensitivity analysis algorithms; (d) assign a cost to the network based on several criteria including the cost to fitting the dynamical experimental data; (e) those networks with the minimum cost are deemed highly probable networks; and finally; (f) billions of hypotheses are weeded out and a subset of hypothetical networks are used to predict biological output. The final output of the methodology is an ensemble of biological networks and their corresponding simulation output that approximate output of the original biological network used for prediction.

The kinds of things predicted might be new biological pathways that describe the functionality of new gene and proteins and their interactions. The function of new genes and proteins beyond what has been characterized in the literature or what is known, such as additional interactions that a particular gene or protein might under go. The methodology can also predict the mechanism of action of a drug or chemical compound. In this case a researcher in the drug discovery field may have a drug discovered via chemical compound screen that gives the desired biological output, but may not know what genes or proteins it is hitting. Via the Computational Hypothesis Testing platform, he or she can treat the drug as an unknown component with unknown interactions and discover the possible genes or proteins it interacts with. Finally, by iterating through various network topologies and generating an ensemble of networks that is a better fit to the data than the original input network, one can then simulate the behavior of this ensemble and use it to predict the phenotypic outcome of the cell and corresponding molecular concentrations. This could be a better predictor of the cell or biological system than the original core network simulation that has not been fit to experimental dynamical measurements in a rigorous way.

Two of the main things to account for when applying this methodology are the assignment of cost and the ability to iterate through a number of hypotheses in an efficient manner. The number of hypotheses available is enormous and cannot be exhaustively studied. Even ten independent hypothetical reactions will lead to approximately a thousand different architectures. It is therefore crucial to use statistical means to incorporate large numbers of hypothetical interactions at a time in the network being tested, and to keep track of the synergies between different hypotheses in matching experimental data. Such a statistical approach can also incorporate the case where there are contradictory functions ascribed to a biological component. Below we describe the assignment of cost to the biological network as well as sensitivity analysis and optimization methods that determine the cost to fitting to a given data set. In addition, we describe a Bayesian scoring algorithm for generating the pool of networks from the probable reactions database and the cost criteria. This allows for the ability to efficiently test out the billions of hypothesis that might be represented in a probable reactions database consisting of a few thousand or more possible hypothetical reactions.

1.5.2 Parameter Optimization

Initially, when a core simulation is constructed a lot of the parameter values are not known. Some can be gleamed from the literature, but most are usually given initial estimates. Parameter values can be identified that better fit the experimental data given a network with some initial parameter values using optimization methods. These methods require the computation of an objective function that can be of the form:

$${CF}^{ k} ( p ) = {\sum }_{i , j} \frac{{( {Y}_{data}^{i , j} - {Y}_{simulation}^{i , j} )}^{2}}{{( {σ}^{i} )}^{2}} ,$$

where p represents all parameters in the simulation such as kinetic rate constants and chemical concentrations, the numerator is the deviation from the simulation to fitting the experimental data, and the denominator the error in the experimental measurement. This basic cost function can also take on other forms such as those that would incorporate error in the time dimension or for data structures with various statistical distributions. This accounts for the deviation of the simulation output from one data set. One usually tries to compare the network output to several data sets. In fact, the more data sets under as many conditions as possible, the better. For each network iteration, one minimizes the global cost function over all possible experimental conditions k given by:

$$CF ( p ) = {\sum }_{k} {CF}^{ k} ( p )$$

The types of data that can be optimized over in a dynamical simulation are: dynamical measurements of the components and chemical species in the simulation under various perturbations and measurements on phenotypic response in the system under various conditions. Conditions can include the cell or biological system under different growth and or cytokine media, with the addition of a chemical compound or drug, a knock out of a particular gene, knock on the RNA level using RNAI or antisense methods, . . . etc. The model is then fit to data by choosing different vectors p of parameter values, integrating the system, then using the simulation to evaluate CF(p). This is iterated until an absolute minimum is achieved. Networks with minimal cost are the ones kept and iterated through the Network Inference Algorithms.

Optimization algorithms encoded in the DigitalCell environment include local and global methods.

Local Minimizers: Local minimizers (which are usually gradient based) start from an initial vector in parameter space and attempt to make “downhill” moves towards the nearest local minimum. The local methods provided by DigitalCell are a Levenberg-Marquardt method (see [12]) and a Sequential Quadratic Programming (SQP) method which is part of the NAG Fortran library. Both these methods require that the cost function be written as a sum of squares as in Eq. (1). Other local methods that are slated for addition to DigitalCell are the Nelder-Mead (simplex) method (see [49], [36]), which does not require any derivatives, and a Nonlinear Programming (NLP) method from the NAG library. The NLP method is gradient based, but does not require that the cost be a sum of squares.

Global Minimizers: In contrast to local methods, global minimizers search the entire parameter space in an attempt to find the lowest possible cost. Global methods can be either deterministic (such as Branch and Bound) or stochastic. DigitalCell provides two global methods, Simulated Annealing and a (μ, λ)-Evolutionary Strategy, both of which are stochastic. Both these methods have been parallelized using MPI. Note that in general, searching the parameter space thoroughly is very difficult when there are a large number of parameters. As a simple example, suppose that each parameter can have only two possible values. If there are five parameters, then the total number of different parameter vectors is 25=32, but if there are 100 parameters, the total number of different vectors is 2100 which is greater than 1030. If computing the cost for one set of parameters takes only one second, it would take approximately 1023 years to test all the different possibilities.

A global method must be able to make uphill moves in order to avoid becoming trapped at a local minimizer which is not the global minimizer. In Simulated Annealing a new parameter vector is formed by generating a random value for each parameter. This value is generated according to a “parameter temperature”. At high temperatures, the parameter values are approximately uniformly distributed across the entire range, and at low temperatures, they are approximately distributed according to a double exponential distribution whose mode is the current parameter value. The cost is computed for the new parameter vector. The new vector is always accepted if the cost is lower. If the cost is higher, the new vector is accepted or not depending on another temperature. At high temperatures, the vector with higher cost is more likely to be accepted. As the algorithm runs, the temperatures are gradually lowered. For slower cooling schedules the algorithm performs a more thorough search.

In the (μ,λ)-Evolutionary Strategy μ is the population size and λ is the number of offspring produced at each generation. To produce a new parameter vector, parents are chosen randomly from the current population. Each parameter value is determined by comparing a random number to a “mutation frequency”, and either generating a random value for that parameter or taking the value of the parameter from one of the parents. The objective function is evaluated for each child, then the μ best children are kept to form the population at the next generation. This method has been shown to perform very well compared to other stochastic global methods (see [46]).

An example of how a global optimization algorithm works is shown in FIG. 14 where the plot figures shows simulation output in solid lines and experimental data points with error bars. The simulation is of the G1-S module which is inputted into the optimization engine along with the experimental data. At the start of the simulation, the parameter values (kb, ku, kp, chemical concentrations, . . . ) were given initial estimates. The initial estimates are such that the solid simulation line does not match the experimental data points. Then a global minimizer is called. The global minimizer varies the parameter values as described above, simulates the system and re-computes the cost. It continues to do this until the simulation curves match the data points. This illustration is shown in FIGS. 14-17.

An important aspect to being able to apply this methodology to large-scale models is to improve the efficiency of the global optimizers since for each iteration over possible network topologies the parameter values have to be constrained to fit the experimental data. Both in terms of the number of parameters they can handle and the amount of time it take. The optimizers in the DigitalCell are encoded on parallel machines. This can be done by dividing the computation of the sampling over each processor. Efficiency improvement roughly scales with the number of processors. Another strategy to use for the optimization, is to optimize each module separately then connect the modules and optimize of the entire cellular network. A typical module would contain roughly 30-100 components and roughly 150-500 parameters. The optimization algorithms need to be able to handle models of this size in a quick manner in order to be able to sample through the billions of hypothetical network strucutures. Another way to handle the large number of parameters is to use sensitivity analysis algorithms that allow one to determine which parameters are the most sensitive to fitting the data. The optimizer can then focus its search on those parameters thereby reducing the number of parameters over which it has to search over.

1.5.1 Cost Assignment to Network Topology

In order to weed through the various hypothetical network possibilities efficiently and also generate biologically relevant netoworks, one must consider other cost criteria besides fitting to the actual experimental time course measurements. These costs are evaluated separately for a variety of criteria (not limited to this list):

(1) robustness against random mutations deleting interactions, or activating interactions that are naturally suppressed, (2) insensitivity to parameters in a probabilistic sense, so that a randomly selected parameter is likely to not require fine tuning in order to match experimental data, (3) insensitivity to experimental uncertainties, in that parameter estimates do not depend sensitively in general to small variations in experimental measurements, (4) approximately scale-free architecture, based on a GNS patent (pending) showing that scale-free networks are less likely to exhibit chaotic behavior, (5) overall bioinformatics cost, calculated from the likelihoods obtained from the probable links database, and finally (6) the cost to fitting the experimental course data.

1.5.2 Bayesian Scoring Algorithm for Network Topology Selection

A specific embodiment for a method to test out the various hypothesis stored in the probable reactions database is a is a Bayesian scoring algorithm. Given a network, consisting of some well-understood biological interactions, and some hypothetical interactions, we want to score the value of the hypothetical interactions in fitting the experimental data. Suppose we wish to classify a hypothetical interaction in terms of either improving fitting to the data (I), making the fit to the data worse(W), or leaving it unchanged(U). We assume that we have a list of network attributes that we can compute (e.g.a partial list is contained in (1) through (5) above), which for simplicity of exposition we will assume are either true (T) or false (F), and we have previously specified the probabilities P(attribute |class) for each of the attributes. We will return to a discussion of how to specify these probabilities later. We start from the a priori expectation that a new hypothesis could be in any of the three classes. The aim is to refine this a priori probability with respect to computed properties using Bayes' theorem:

P(A|B)P(B)=P(B|A)P(A)

to compute

P(class|attribute)=P0(class)P(attribute|class)/(P(attribute)=1)

for each class, and then renormalizing the left hand sides by

Σclass P(class|attribute)

to obtain posterior probabilities. We then use these probabilities as the prior probabilities P0(class) for the next attribute on our list, until we have exhausted all the attributes. The final P(class) probabilities are the numbers on which we base our future sampling of this hypothesis for inclusion in other networks as we continue sampling in the space of networks.

This iterative refinement of this pool of networks leads to certain hypotheses exhibiting falling into the improving' class, and the union of these hypotheses with the well-understood core of the network is our best prediction according to these multiple criteria. In the GNS architecture, a pool of networks is simulated in order to make predictions, and the overall prediction for the behavior of the microbe in a new environment is the cost-weighted sum of the pool of networks' predictions. Thus, networks which mostly comprise hypotheses that are highly likely according to all the five criteria listed above are dominant in the prediction. This is the proposed approach to dealing with the problem of unknown structure and partial observability in the biochemical network.

A last point that must be addressed is the following: Where did the probabilities P(attribute |class) come from? These are parameters in our approach and must be validated against experimentally known facts. These measure the relative importance of the different attributes being computed as good criteria for constraining network architecture. This algorithm can allow for the testing of large number of hypothetical networks in an efficient manner. Again, using the Computational Hypothesis Testing platform one is trying to generate network models that account for the missing information inherent in most biological systems by both inferring parameter values and actual components and interactions. The space of possible interactions is therefore very large and must be efficiently sampled.

1.5.3 Using a Population of Networks to Generate Predictions

As stated in the introduction to section 1.5, the final output of the methodology is an ensemble of biological networks and their corresponding simulation output that approximate output of the original biological network used for prediction.

Predictions are generated using the populations of networks generated as having the lowest overall cost determined by the cost criteria mentioned in section 1.5.2. To predict simulation output of chemical components in the simulation, one can take an average over the predicted time courses in the population weighted by the overall cost. To generate predictions on a predicted new component or interaction, then for a given new predicted component and or interaction that is present in at least one or more of the network, one can determine the number of networks that predict that the component and or interaction needs to be present to minimize the cost. Those components and interactions then have a high probability of being part of the original network in the biological system.

If one only takes the process to the level of parameter optimization, then there is only one network model. However, if there is not enough data to constrain the simulation, then there may be populations of parameters that fit the data (e.g. multiple minima in the global cost function space). The patent “Methods and Systems for the Identification of Components of Mammalian Biochemical Networks as Targets for Therapeutic Agents” discusses methods for weeding out models where more than parameter population can fit the data.

1.6 Experimental Validation and Iterative Refinement of the model

Once predictions are generated, they can then be tested experimentally and used to iteratively refine the model. Predictions can be generated on the expected time course and or static expression of the components in the whole cell model or biological system. Experimental methods that measure mRNA levels, protein levels, metabolites, and their localizations can be used to test the validity of the prediction. Predictions can also be generated on the physiological level and assays that test for physiological changes can be performed to test the prediction.

Whole cell simulation of E. coli

A description of building a complete large-scale data driven simulation of E. coli is provided. The methodology described here is general and can be applied to modeling any bacterial system.

From a Modular Description to a Detailed Description and Then Back

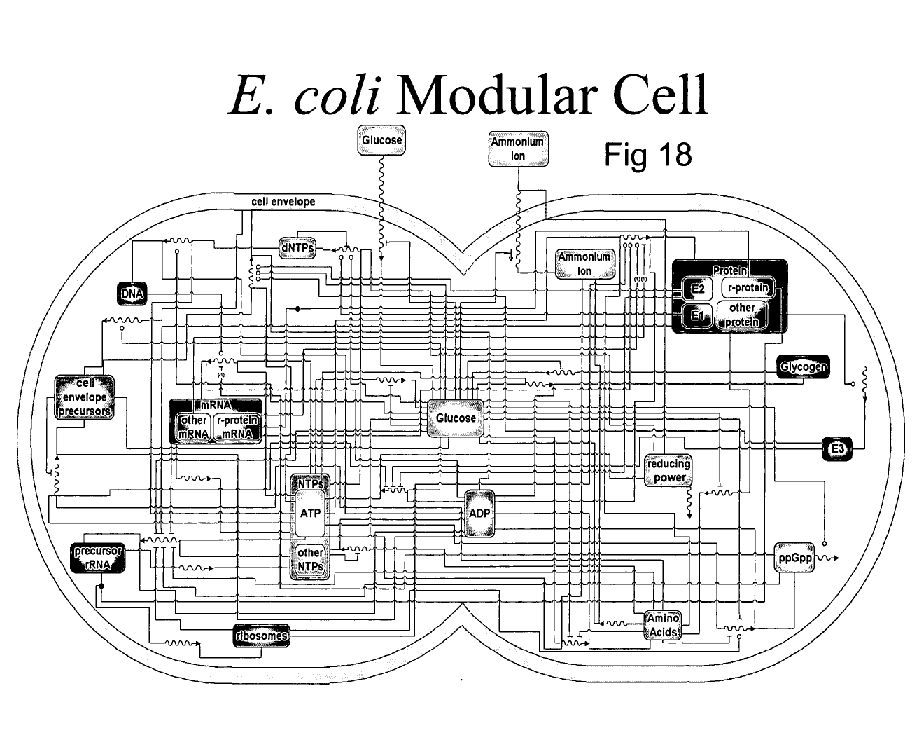

Previous attempts at modeling E. coli on a whole cell level have consisted of identifying major functional modules in the bacteria and using those modules to represent many cellular constituents (see the work by M. L. Shuler et al 1985 and 1991). FIG. 18 shows a representation of an E. coli whole cell module in the Diagrammatic Cell Language that treats various cellular components in a modular form. For example, the module amino acids refers to all Amino Acids, the module Glucose (within the cell) represents glycolysis and the citric acid cycle, cell envelope precursors allude to the lipids and sugars that form part of the cell wall, etc. The model responds to two external signals: glucose and nitrogen. Note that the module glucose in the external environment represents only glucose unlike its counterpart within the cell. Connections between modules are shown in DCL representing the detailed interactions between the modules. The description then translates to a set of differential equations written out for interactions/process in that can be solved within a simulation environment such as the DigitalCell.

The modular whole cell E. coli model predicts physiological outcomes such as cell division, cell growth, changes in cell shape and volume. It includes the processes of transcription, translation, replication, transport, catabolism, and anabolism; the output is dynamical and includes concentrations of proteins, amino acids, nucleotides, envelope components and other bulk cell components

While the modular E. coli model has been shown to predict E. coli response to a number of external and internal perturbations, the model has some limitations. Firstly, this model cannot be used to predict outcome on the molecular level. For example, if a particular transcription factor is knocked out, how will that effect the timing cellular division? A model that can predict phenotypic outcome on the molecular level (perturbations of genes, proteins, and metabolites) is required for current medical and industrial applications since this is the level that perturbations are occurring on. Secondly, The modular E. coli model has not necessarily identified the actual modules in the bacterial cell and can actually breaks down at some point. One needs an approach that can identify the actual modules starting from a model of all of the cellular constituents.

Starting from this basic modular model template, a detailed model is built that scales to include every known gene and protein in E. coli. One can then back track and use the detailed model to identify the minimal number of modules one needs to simulate to describe a whole cell E. coli model. In doing so, one will also identify the minimal gene set needed to account for a completely functional bacteria. The minimal gene set can serve as a basis for engineering new bacteria that contain enough components at their base to account for life, and then include the additional components for engineering bacteria with particular functionality (e.g. bacteria that can degrade human waste).

Creation of the Core Whole Cell E. Coli Simulation:

The goal is to build a detailed simulation of E. coli that includes every known gene in E coli. Such a simulation would respond to Glucos and Nitrogen to predict cellular division and growth, and also to other external cues such as Oxygen, acetate, temperature, availability of salts (Mg, Fe, etc.), inorganic phosphates, CO2, vitamins etc. The table below lists the known pathways in E. coli needed to build a complete E. coli model (the list is growing as new pathways are elucidated). It lists the pathway and end point of the pathway both in terms of final chemical output and physiological output.

TABLE 1.1

| | |
|---|---|
| 1. | Glycolysis-breaks down glucose into ATP and pyruvate. ATP |
| | provides energy for cellular functions and pyruvate is channeled into |
| | the TCA cycle. |
| 2. | TCA cycle-results in energy production and also yields precursors for |
| | amino acid metabolism and cell wall synthesis. |
| 3. | De novo pyrimidine synthesis-produces pyrimidine building blocks |
| | for DNA & RNA |
| 4. | De novo purine synthesis-produces pyrimidine building blocks for |
| | DNA & RNA |
| 5. | Salvage pathway for purine synthesis-produces pyrimidine building |
| | blocks for DNA & RNA |
| 6. | Salvage pathway for pyrimidine synthesis-produces pyrimidine |
| | building blocks for DNA & RNA |
| 7. | Pentose phosphate pathway-Generates reducing equivalents of |
| | NADPH as well as intermediates for nucleotide biosynthesis and |
| | central carbon metabolism |
| 8. | Arginine biosynthesis-synthesizes the amino acid arginine |
| 9. | Methionine, lysine and threonine biosynthesis-synthesizes amino |
| | acids methionine, lysine and threonine |
| 10. | Isoleucine, valine and alanine biosynthesis-synthesizes amino acids |
| | isoleucine, valine and alanine |
| 11. | Histidine biosynthesis-synthesizes the amino acid histidine |
| 12. | Asparagine biosynthesis-synthesizes the amino acid asparagines |
| 13. | Tryptophan, tyrosine and phenylalanine biosynthesis-synthesizes the |
| | aromatic amino acids, tryptophan, tyrosine and phenylalanine |
| 14. | Amino acid metabolic pathways for cysteine, glycine, leucine, |
| | proline-2 months. These end products are used in protein synthesis. |
| 15. | Vitamin and cofactor metabolism. Used as cofactors for metabolic |
| | enzymes. |
| 16. | Cell wall metabolism. Involves synthesis of components of the |
| | bacterial cell wall. |
| 17. | Lipid metabolism, signal transduction systems and transport |
| | processes. Lipid metabolism involves synthesis of fats, signal |
| | transduction pathways conduct information about changes in the |
| | external cellular environment to the cell interior. |
| 18. | Alternate carbon metabolism, energy metabolism and central |
| | intermediary metabolism (synthesis of ppGpp, etc) |

Note that the pathways 1-14 synthesize precursors that serve as building blocks for larger macromolecules, for example, amino acids serve as building blocks for proteins while, as stated earlier, nucleotides (purines and pyrimidines) serve as building blocks for DNA and RNA.

The E. coli whole cell model would include all of the above pathways and would be responsive to the following external cues:

TABLE 1.2

| | |
|---|---|
| 1. | Glucose—glycolysis, the citric acid cycle (TCA cycle), pentose |
| | phosphate pathway |
| 2. | Nitrogen—basically used in amino acid biosynthesis |
| 3. | Oxygen—glycolysis and the TCA cycle, electron transport chain |
| 4. | Acetate—TCA cycle and the glyoxylate shunt |
| 5. | Temperature—there is a different version of the model that takes |
| | temperature effects into account. This model includes heat shock |
| | proteins and predicts changes in reaction rates in response to |
| | temperature. |
| 6. | Availability of metals (iron, magnesium, etc)—Magnesium and |
| | iron act as metals cofactors for enzymes that catalyze metabolic |
| | reactions. |
| 7. | Vitamins—these also act as cofactors for enzymes and are needed |
| | for the catalytic activity of metabolic enzymes. |
| 8. | CO2—Carbon dioxide is generally released into the medium and I |
| | recall having read that this is generally bad in fermentation |
| | reactions because it means you are dissipating carbon flux in the |
| | form of CO2. |
| 9. | Inorganic phosphates—these are used to generate phosphate-based |
| | products such as nucleotides (ATP, etc), NADPH, etc. Phosphate |
| | derivatives are also used in amino acid synthesis pathways. |

The body of biological literature and databases is mined to construct detailed molecular maps of the pathways listed above indicating the interactions between the cellular components (gene, proteins, metabolites, . . . etc.). For each interaction, a rating is given to the validity of the interaction based on experiments used to verify the interaction. For example, for the interaction reversible cleavage of the component FBP to GAP and DHAP, the curator analyzed two experiments and rated them according to the experimental method used. This interaction is then represented in DCL and scaled and expanded on to include other interactions and components in E. coli.

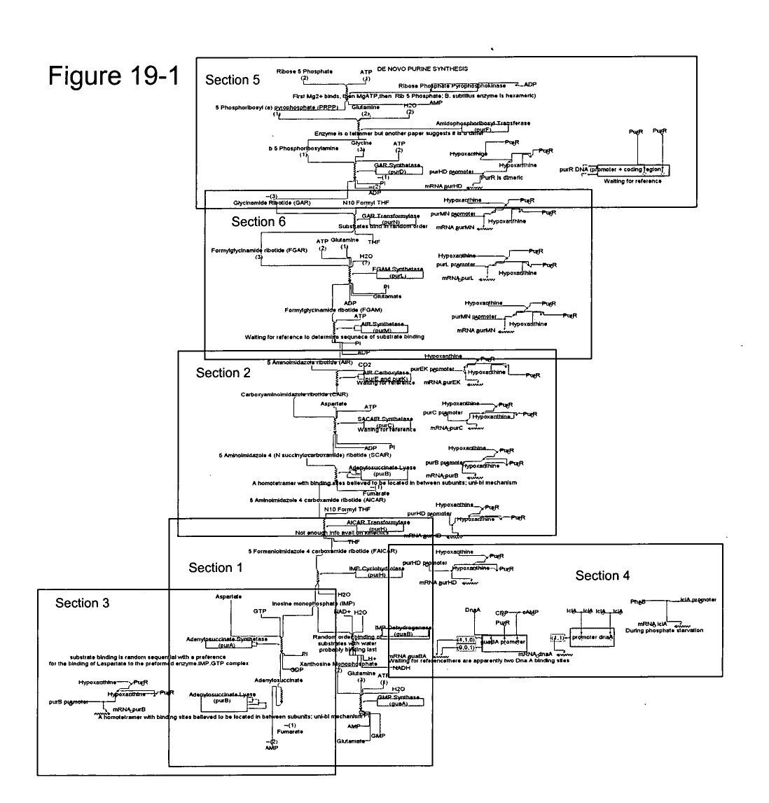

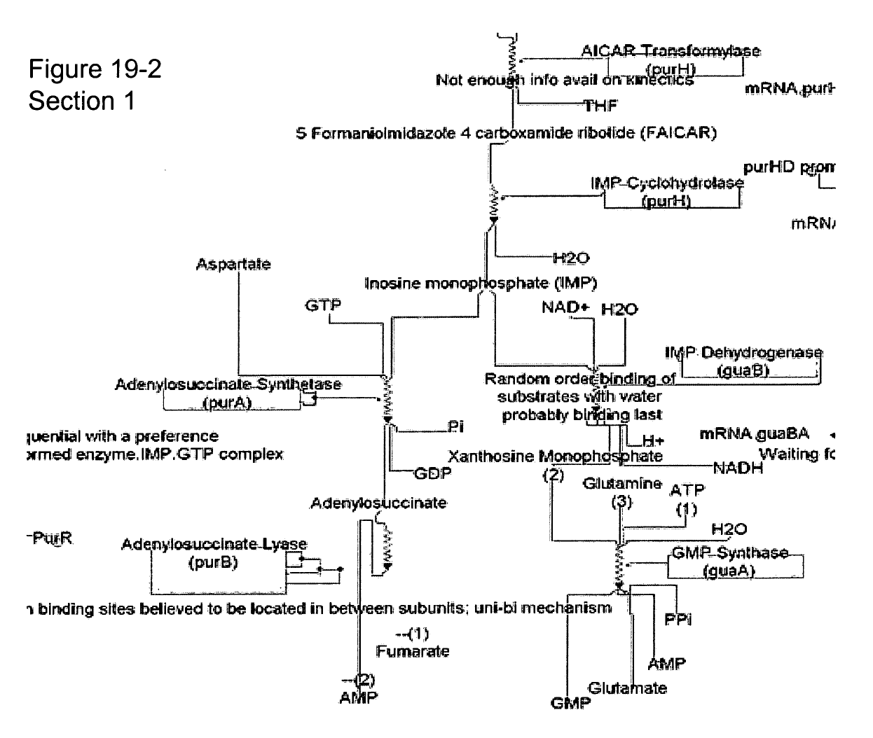

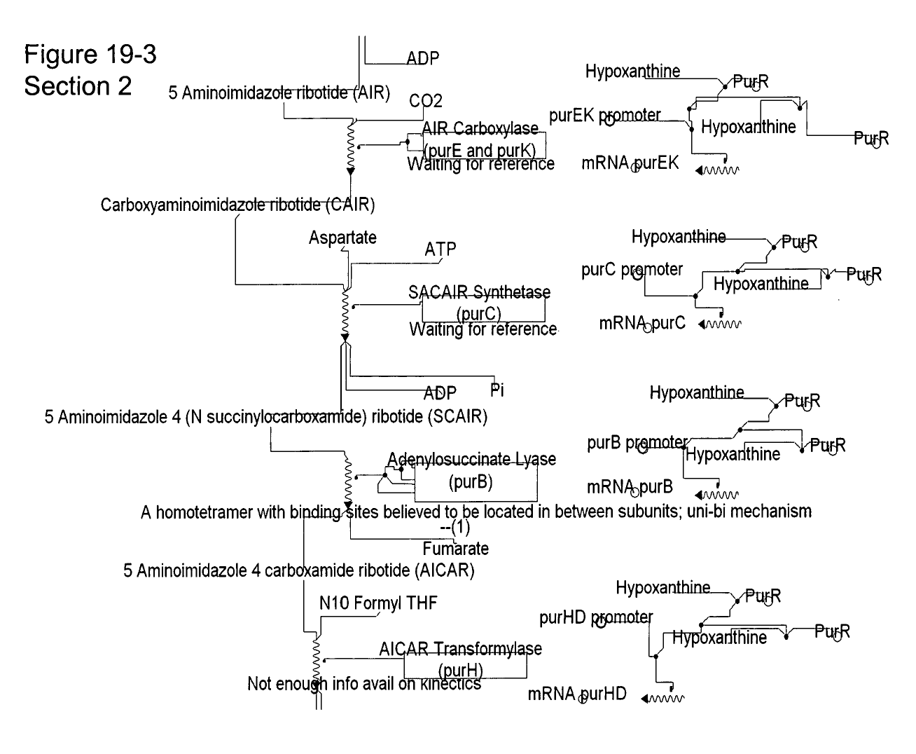

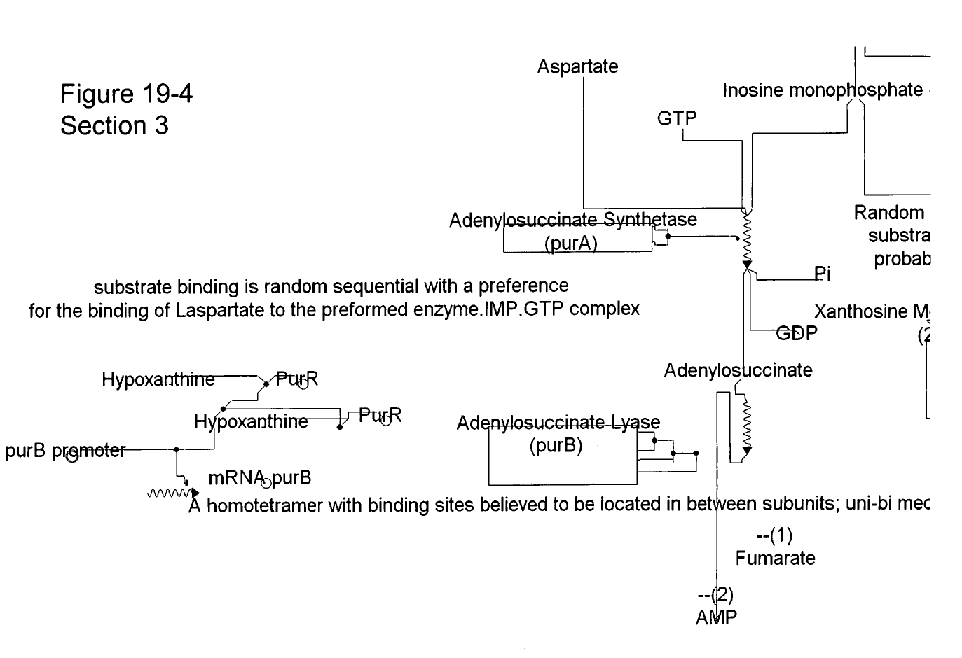

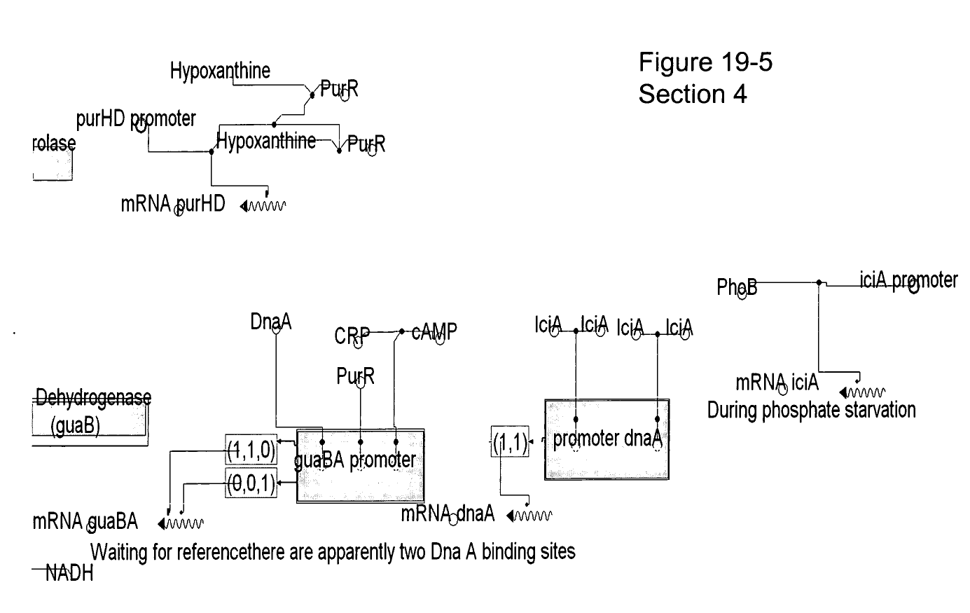

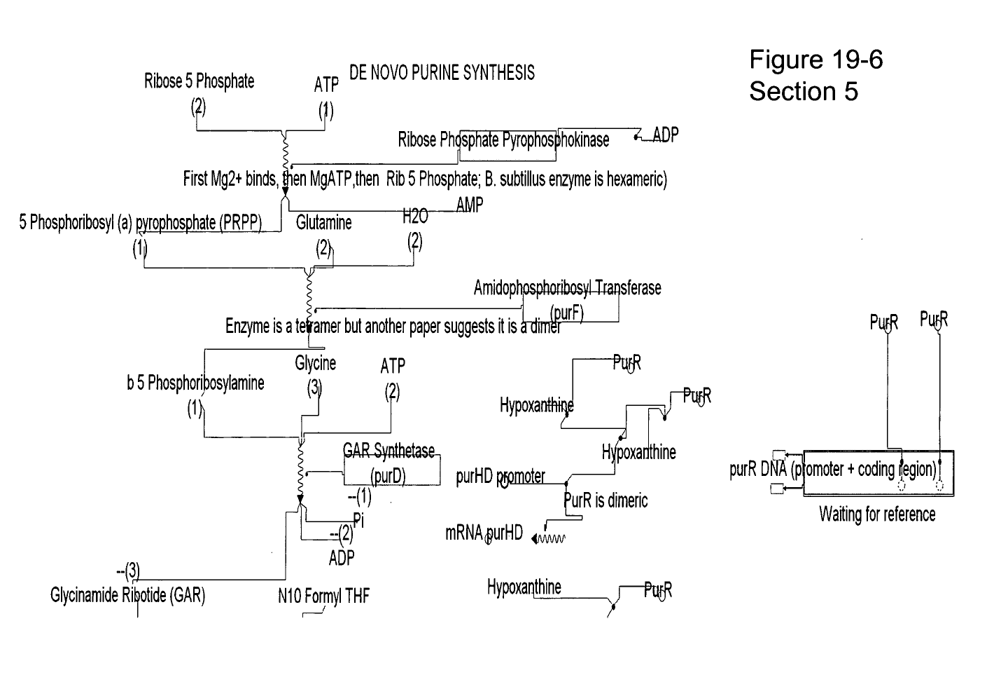

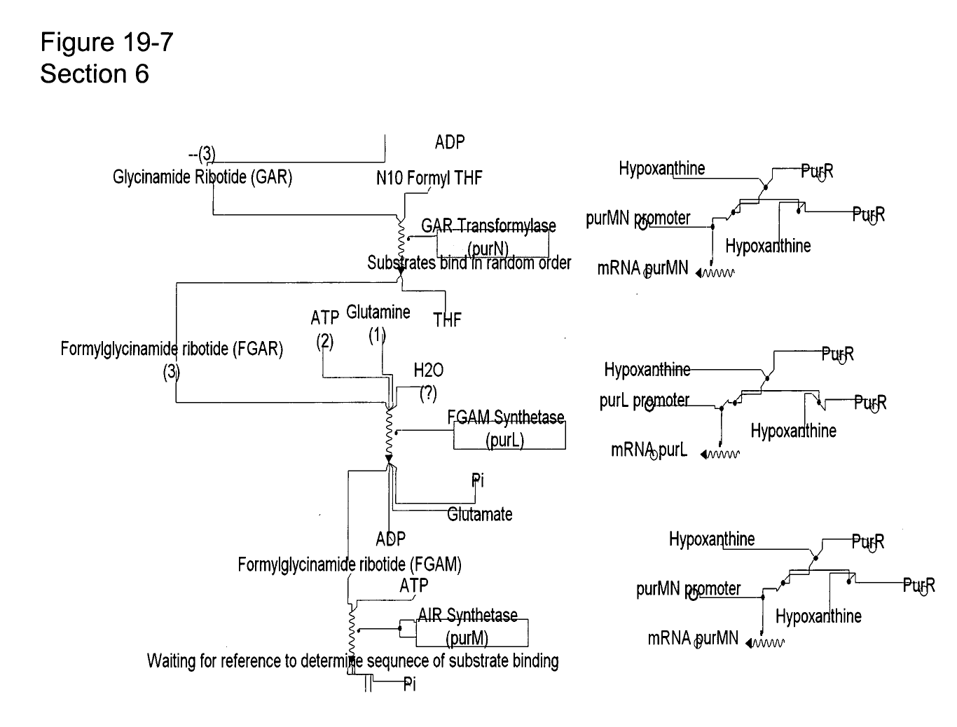

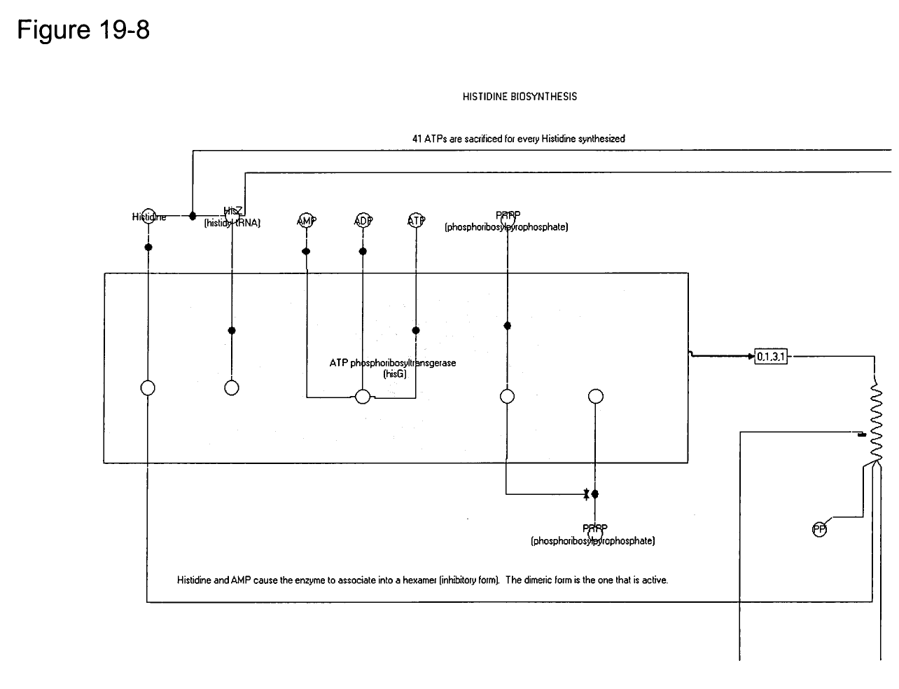

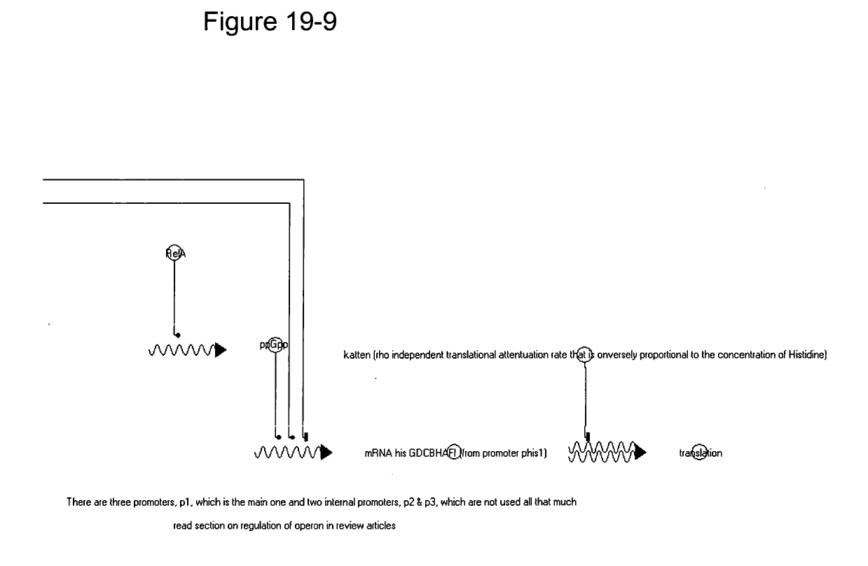

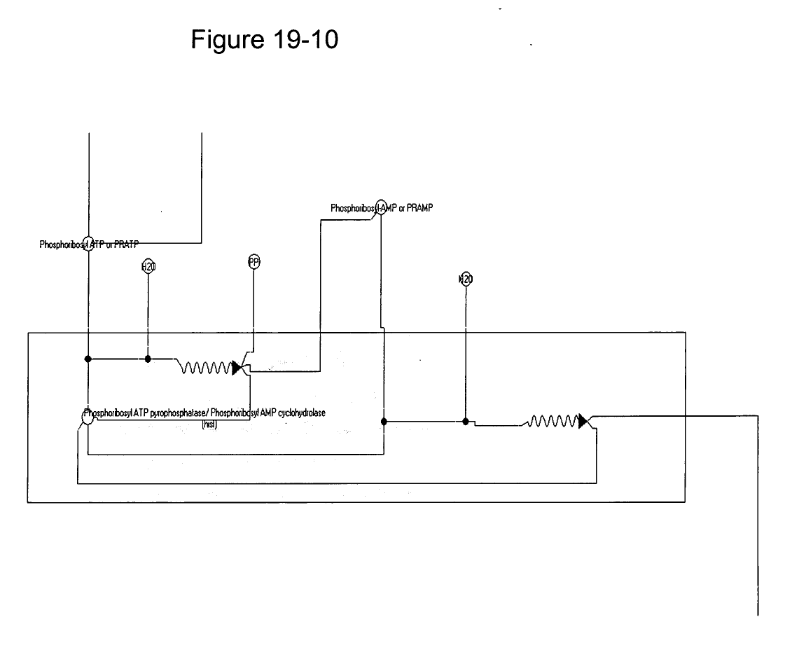

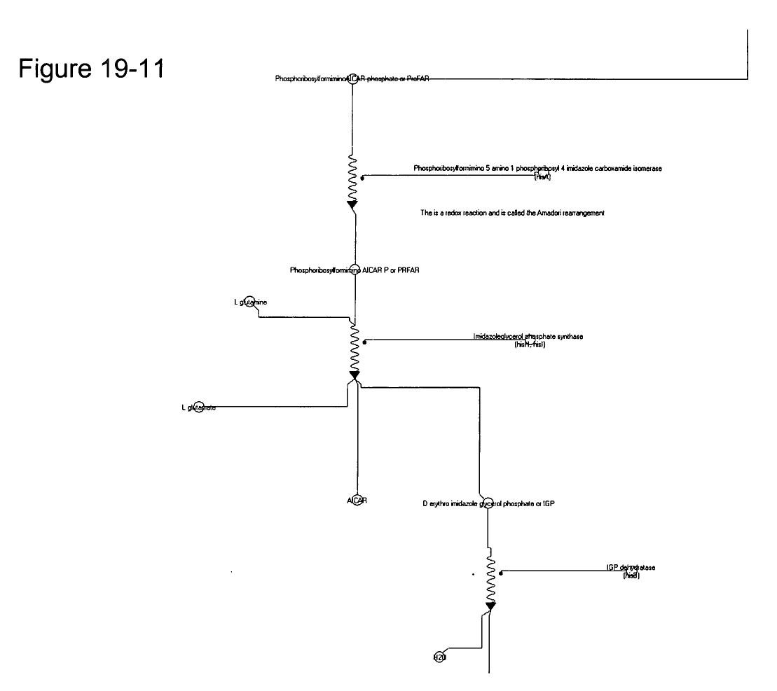

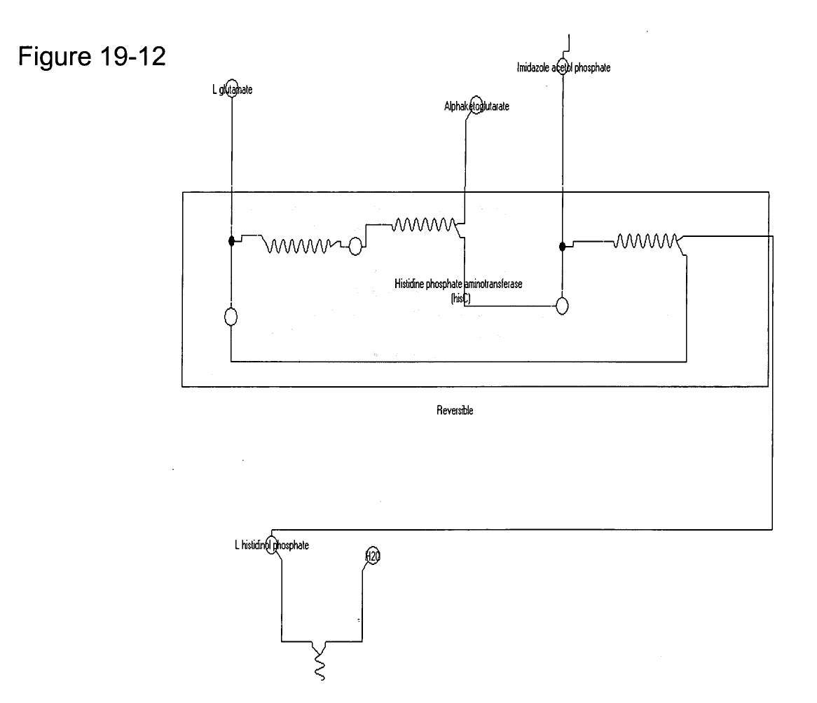

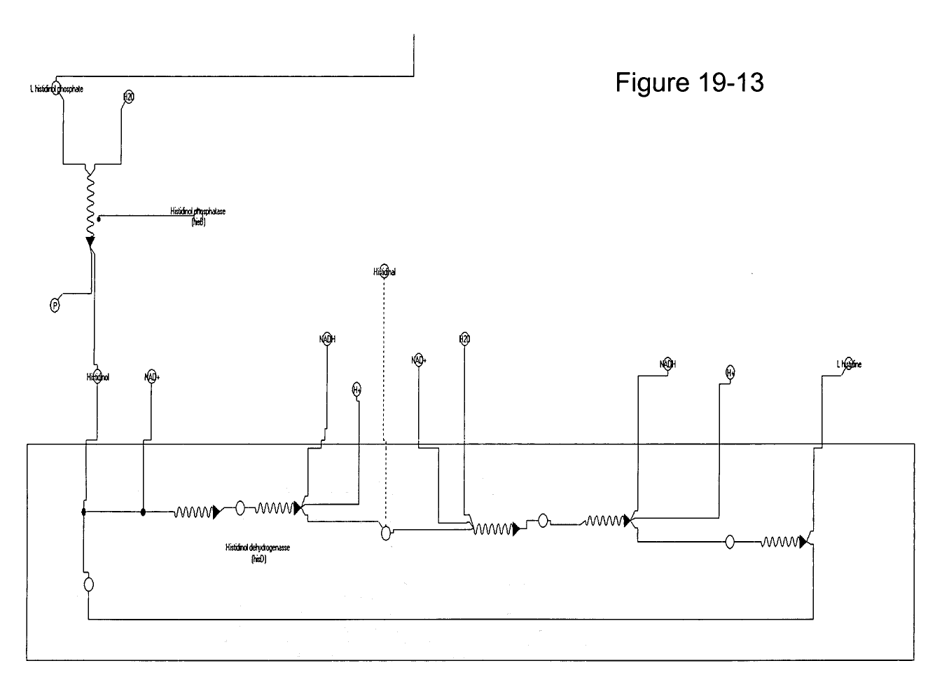

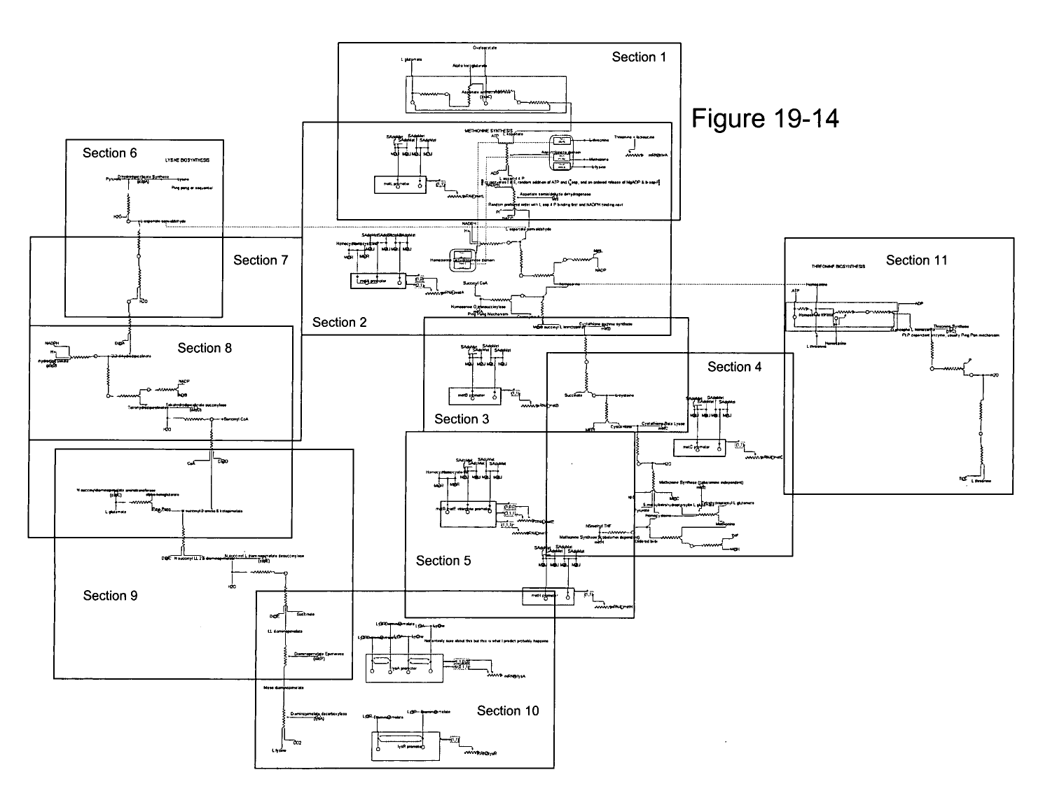

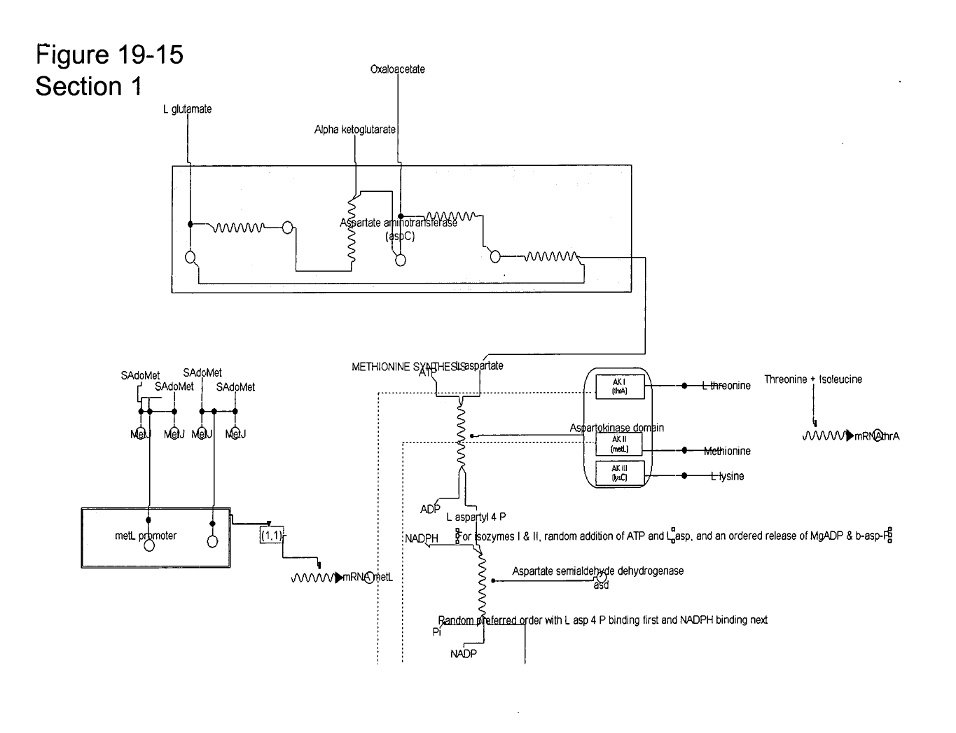

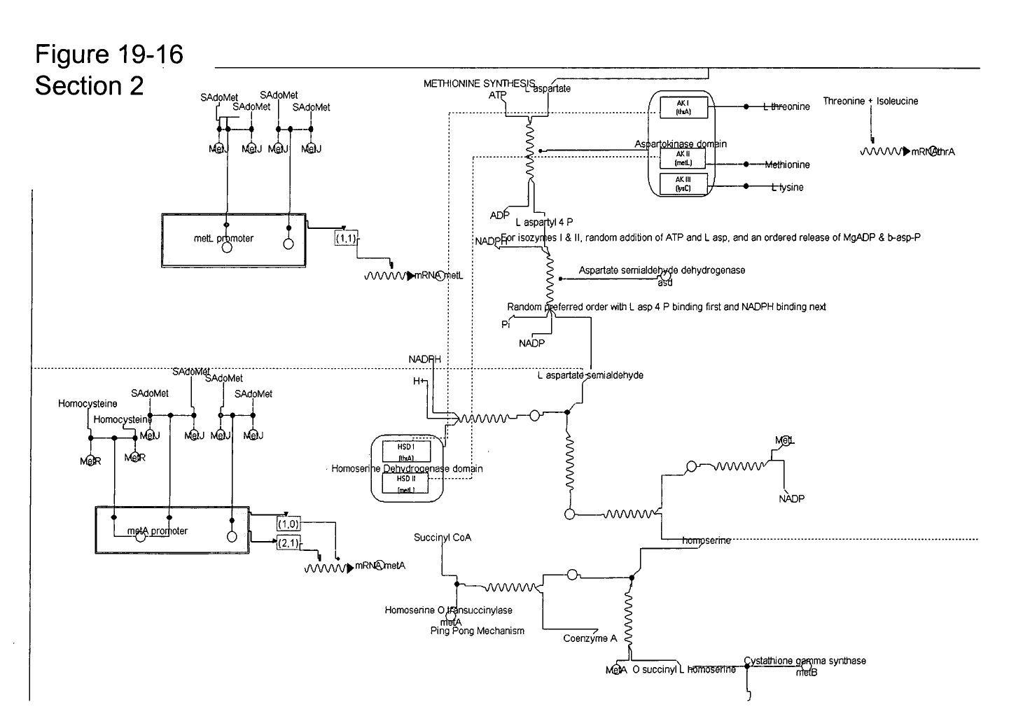

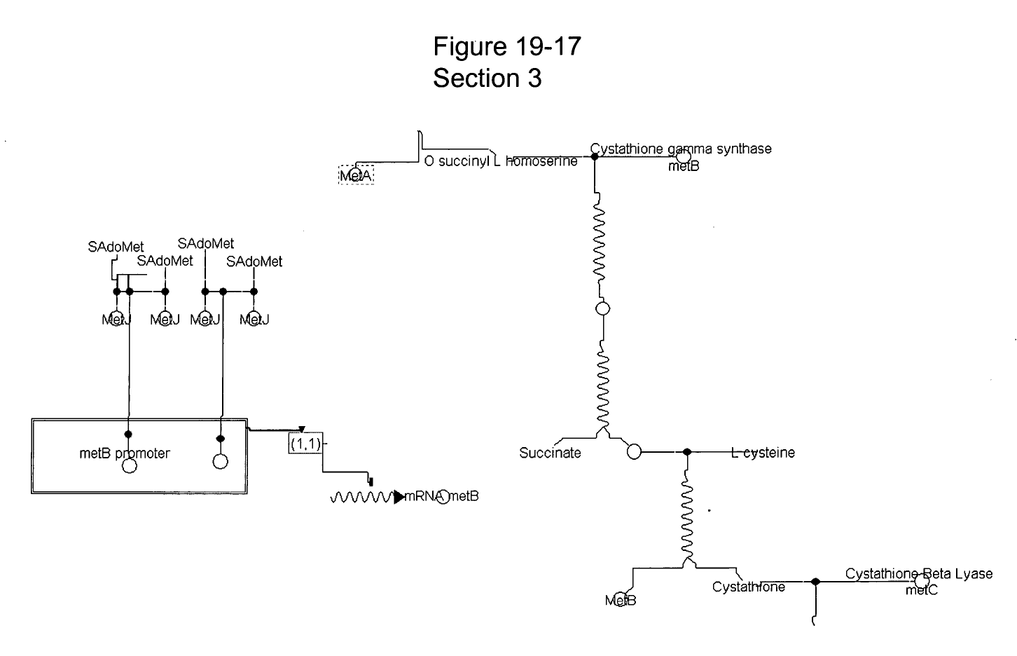

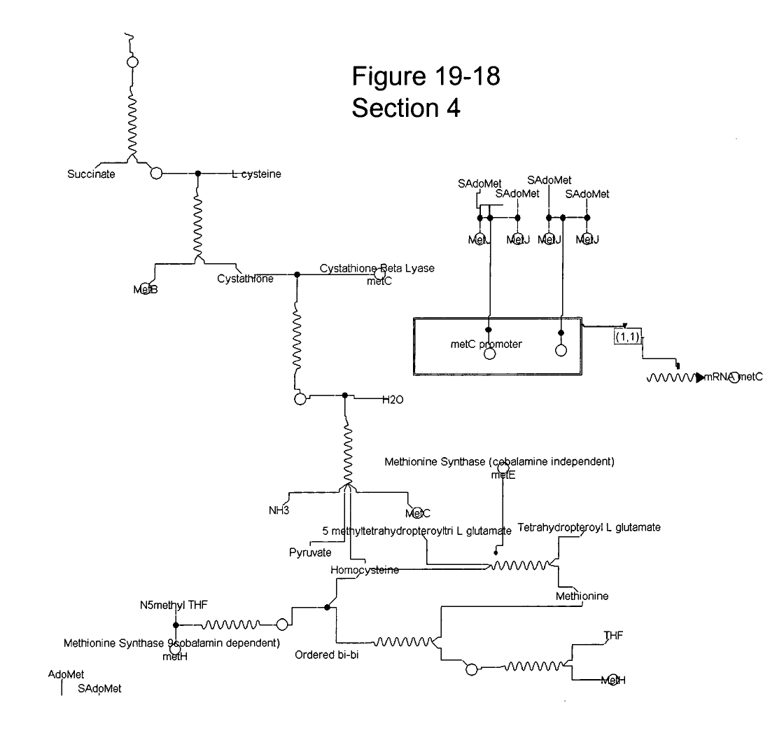

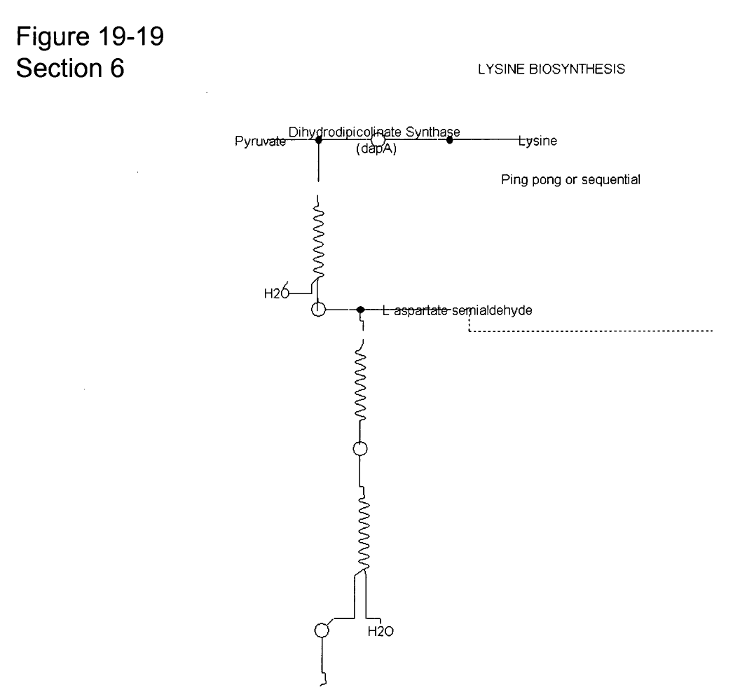

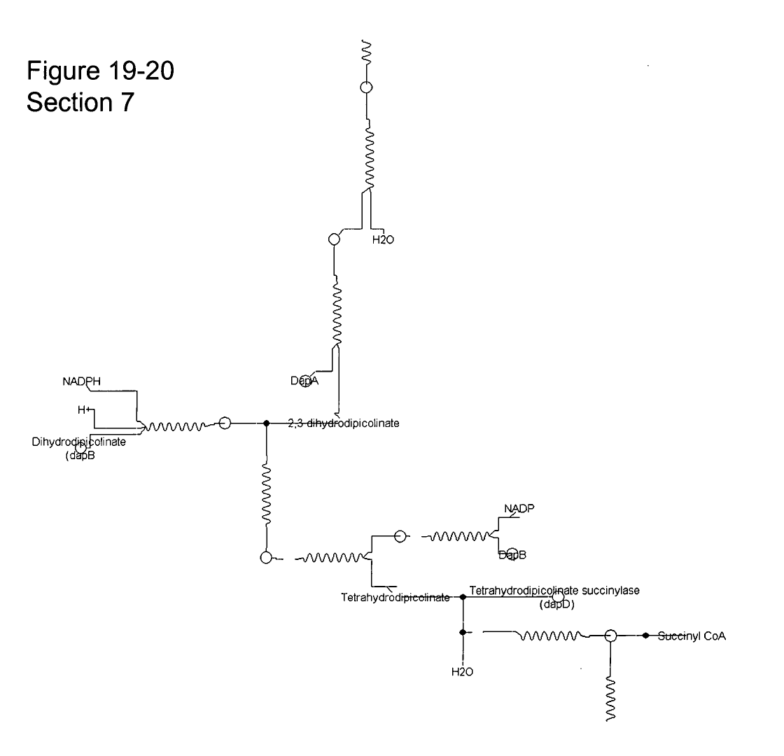

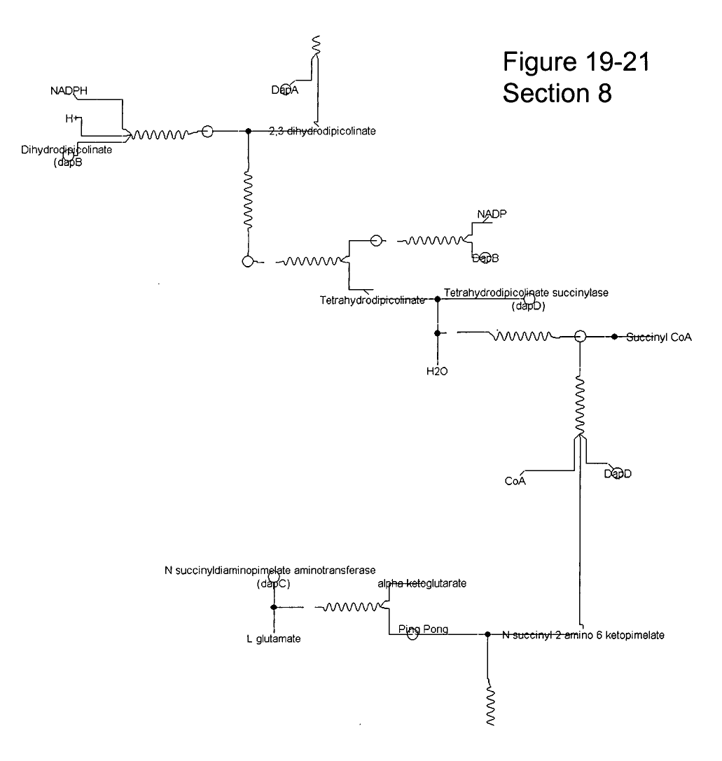

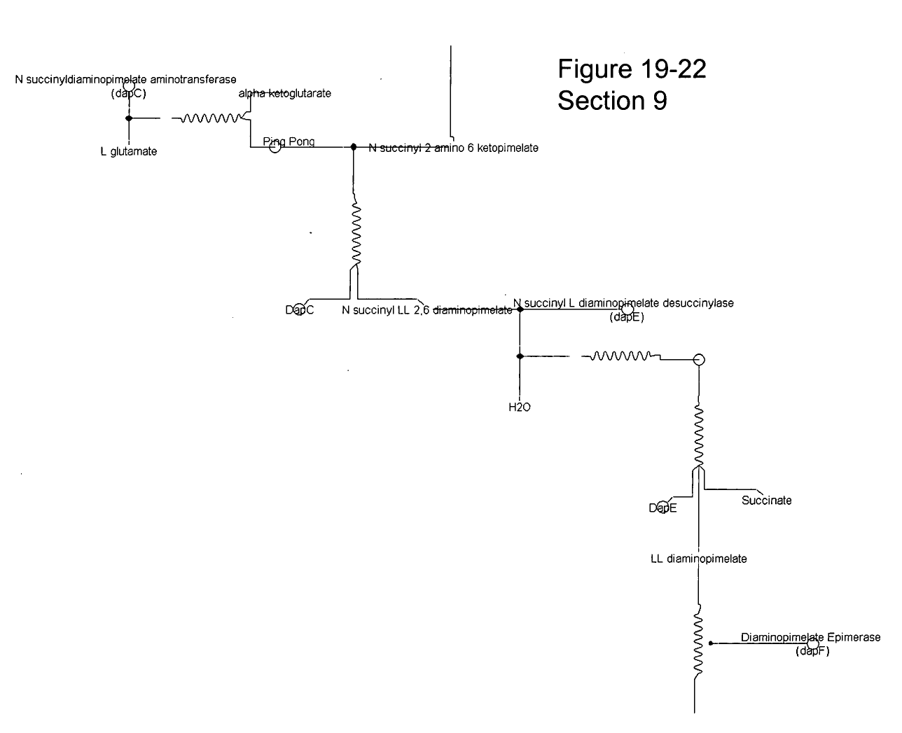

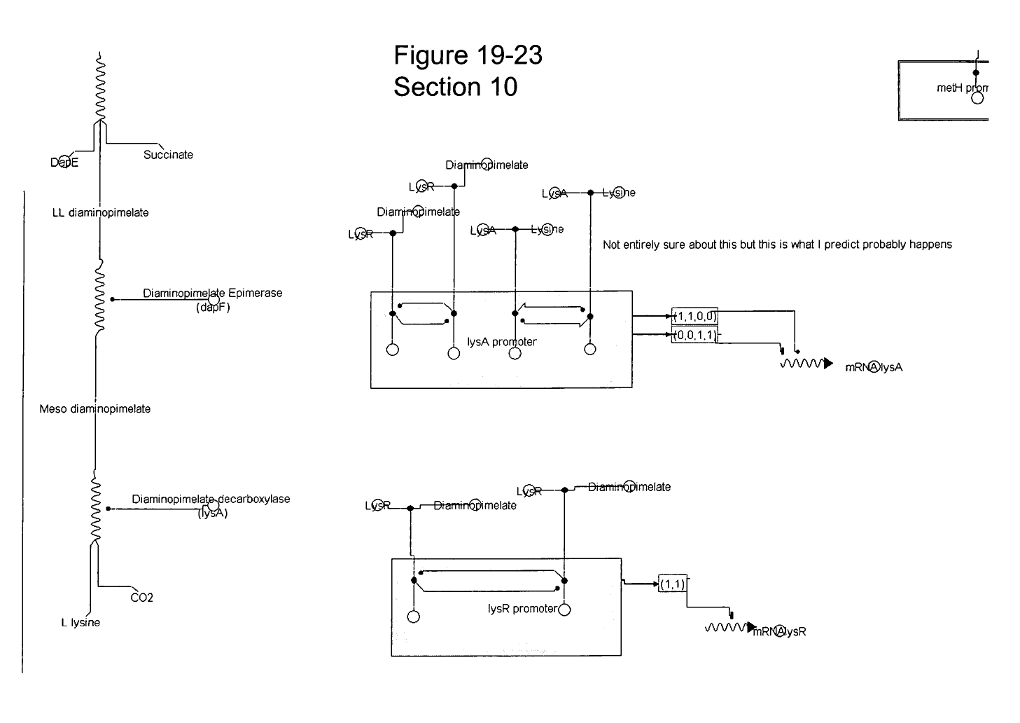

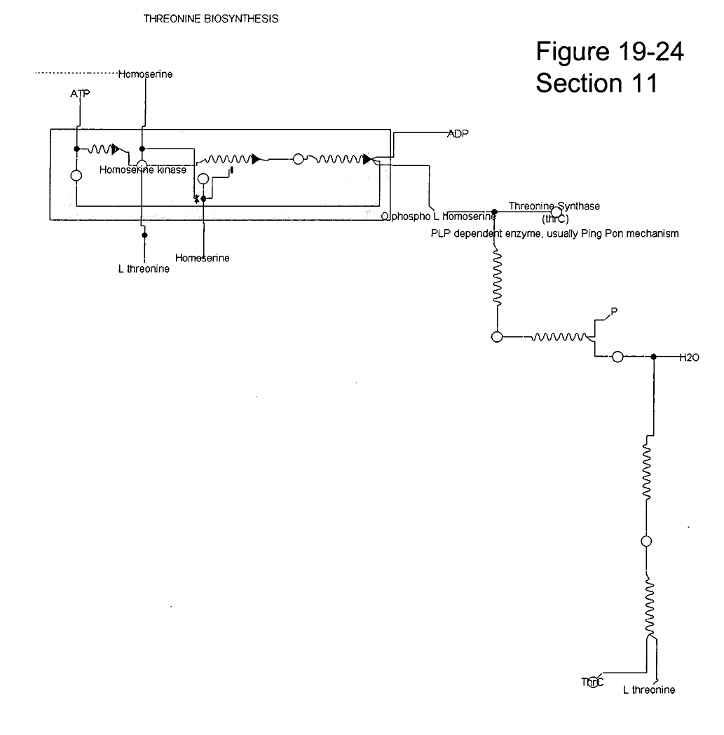

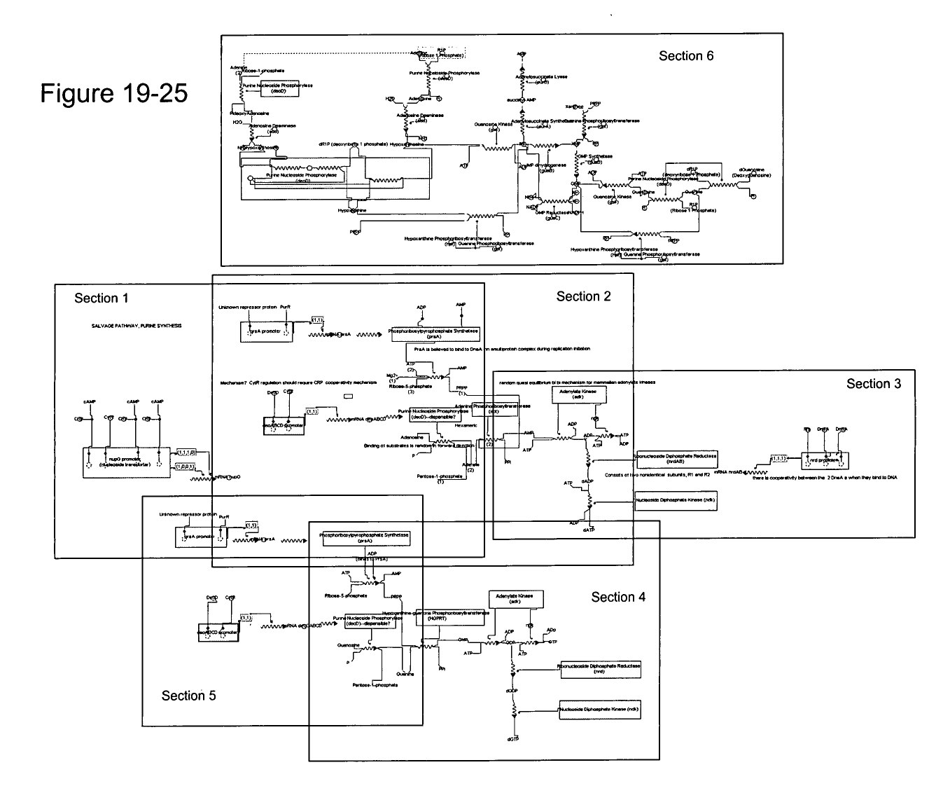

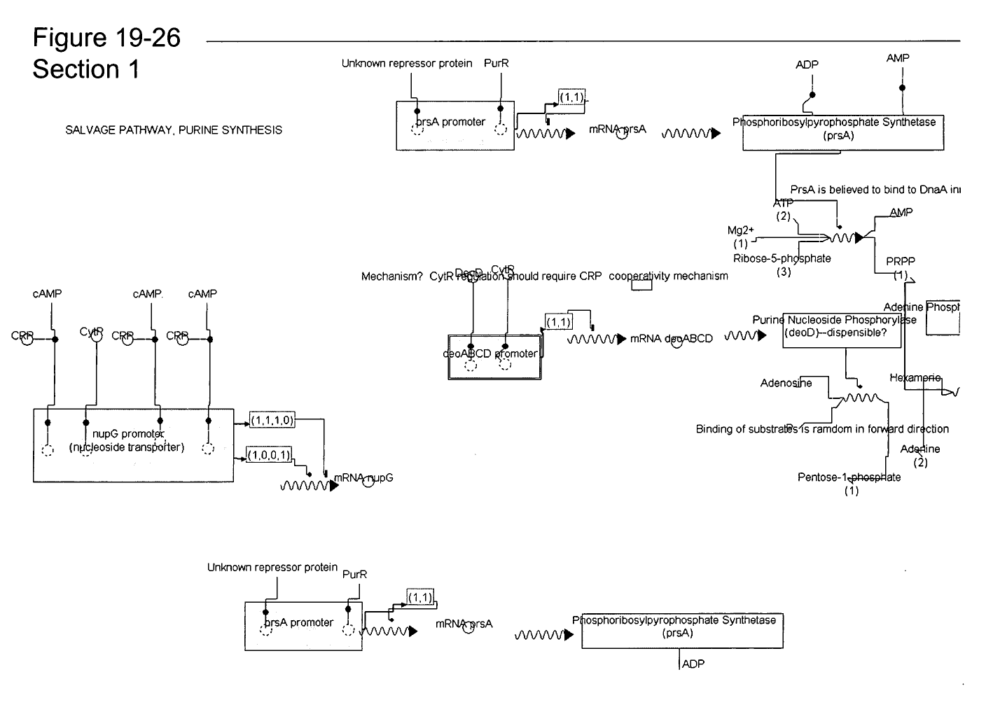

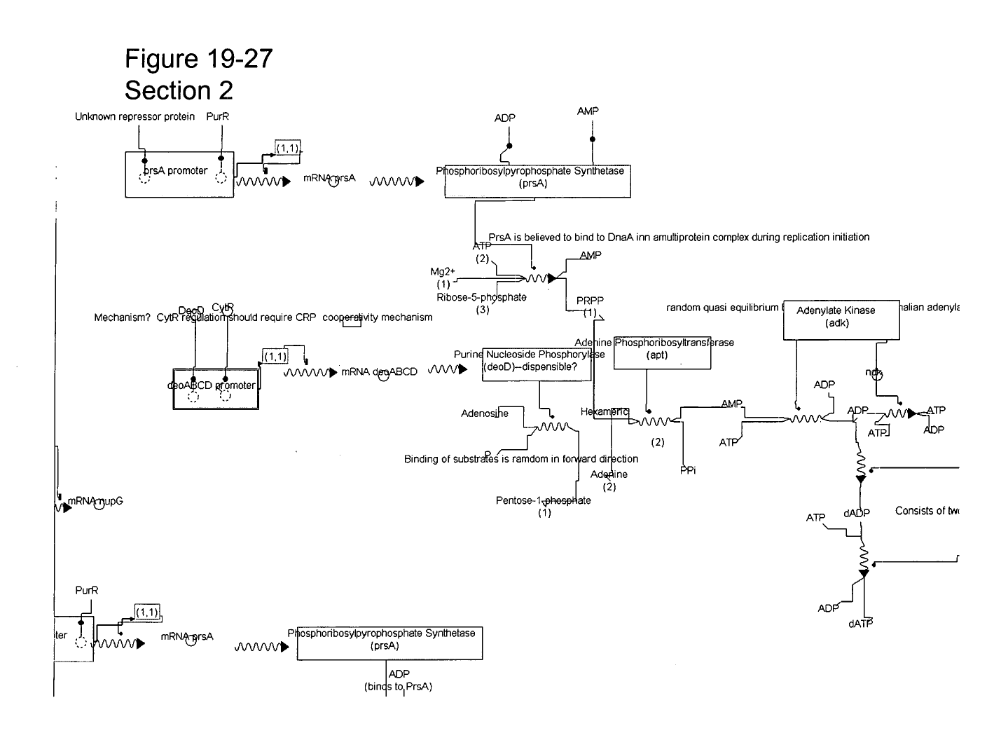

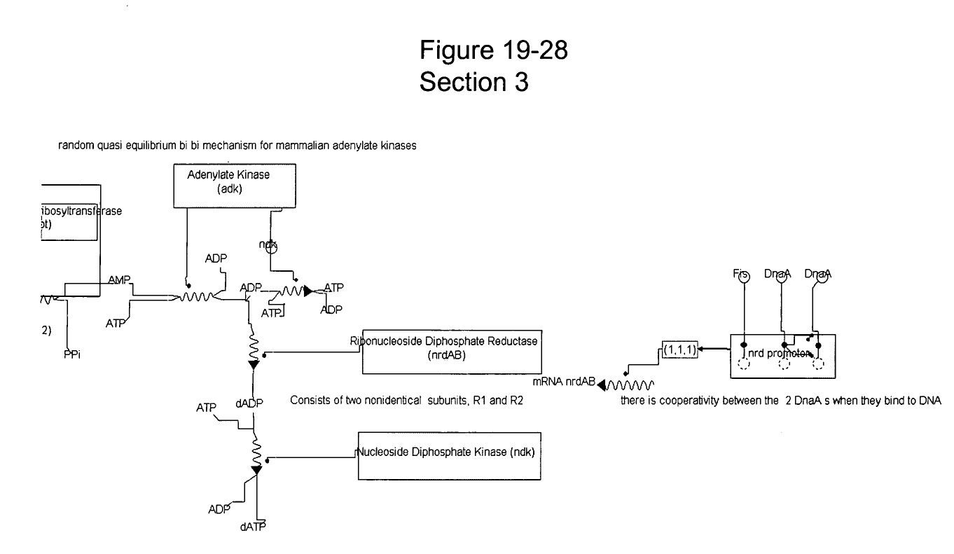

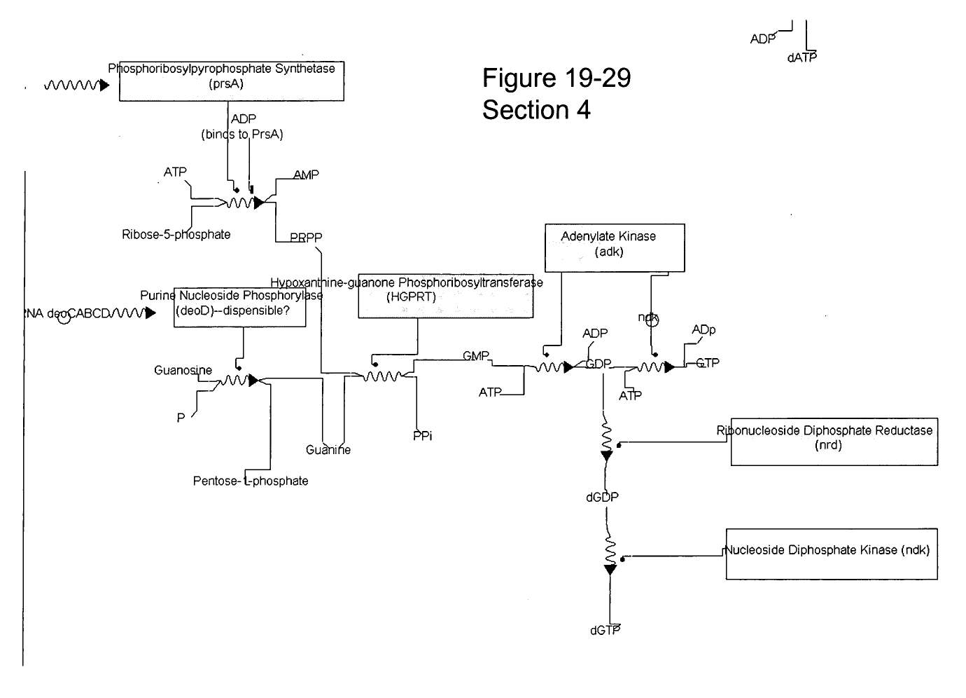

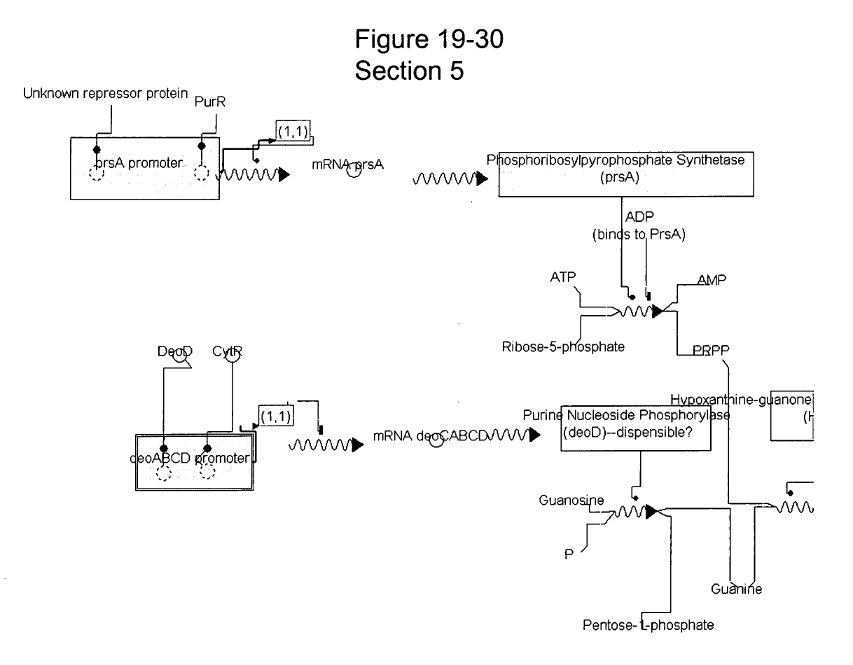

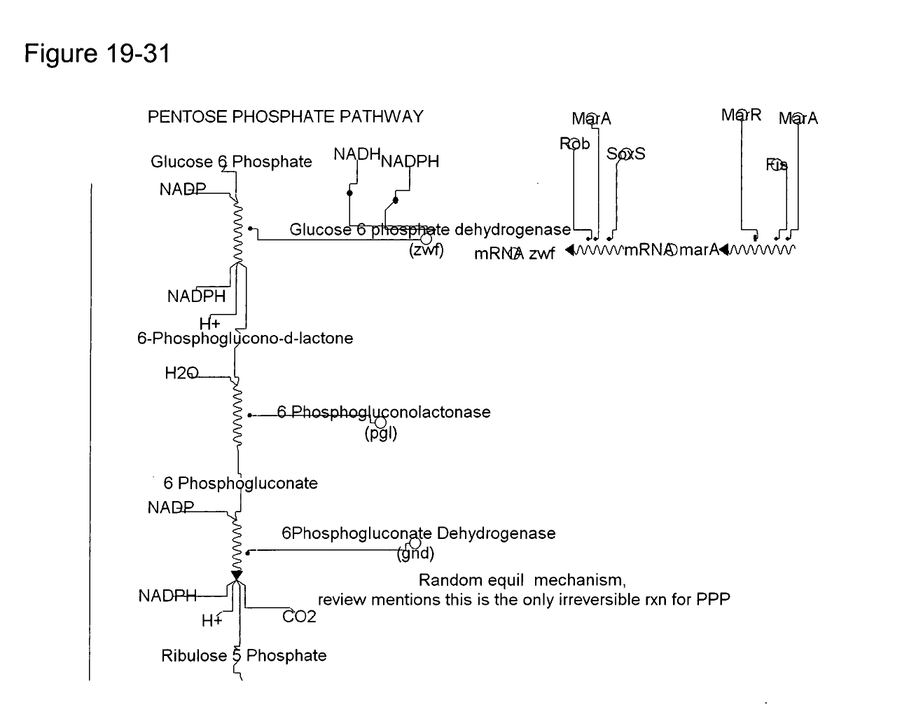

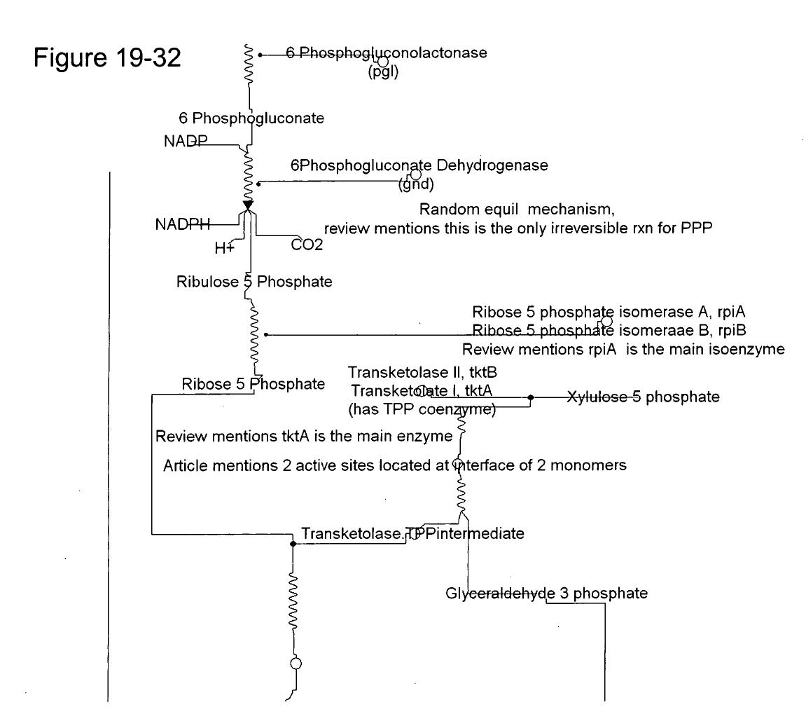

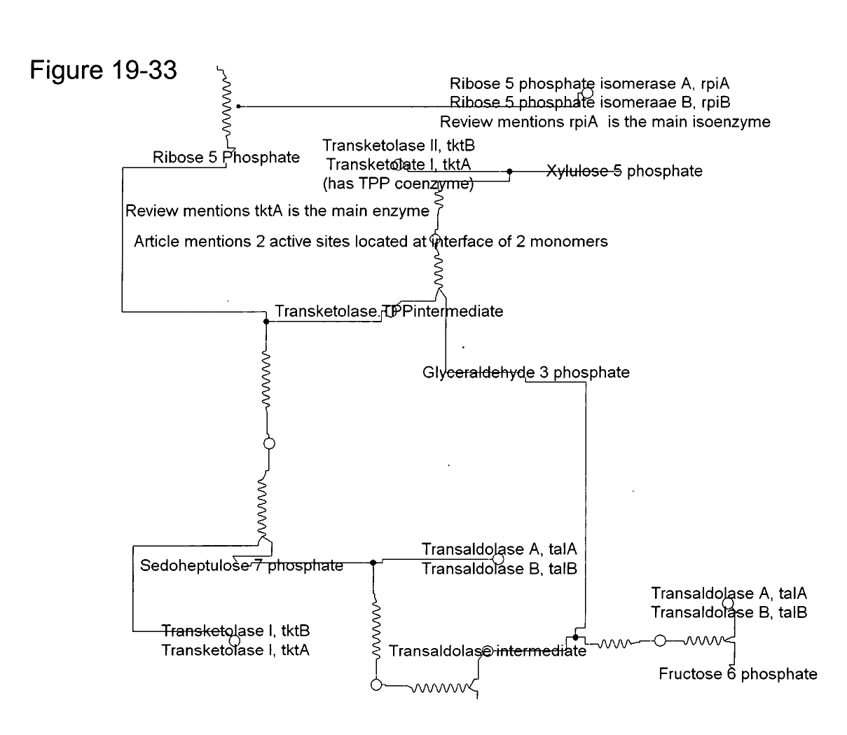

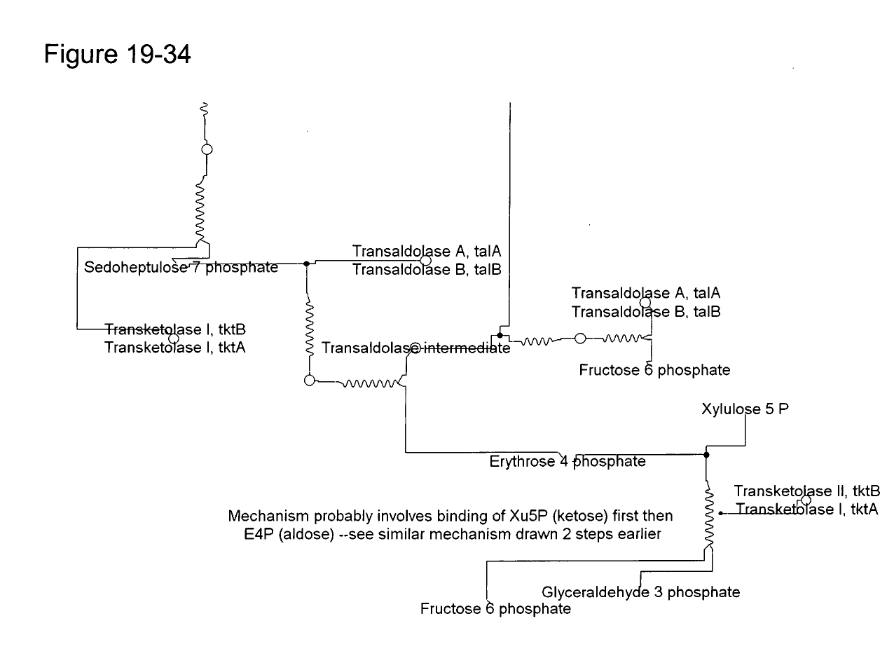

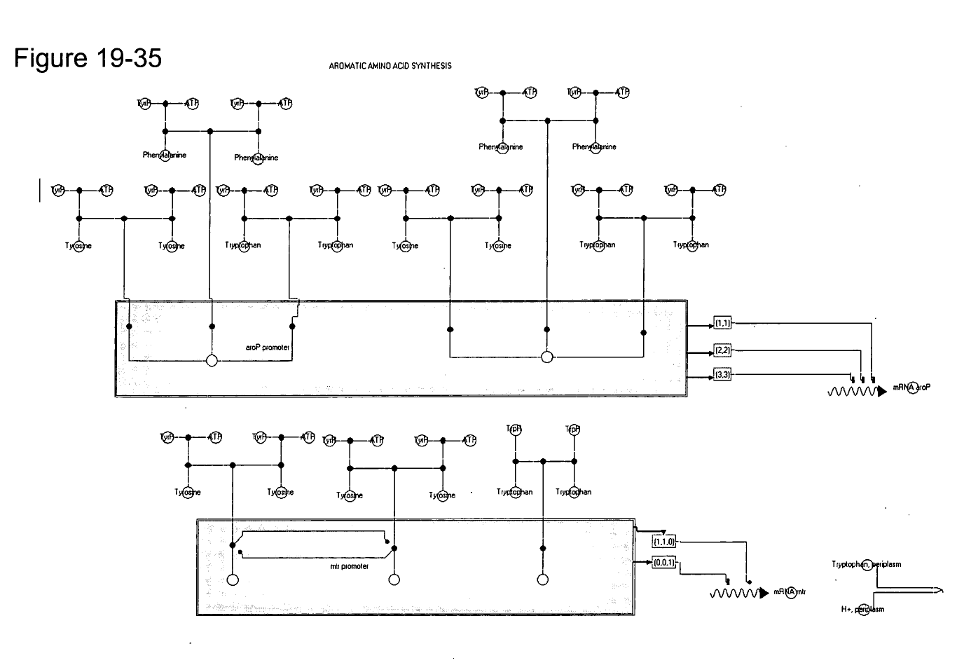

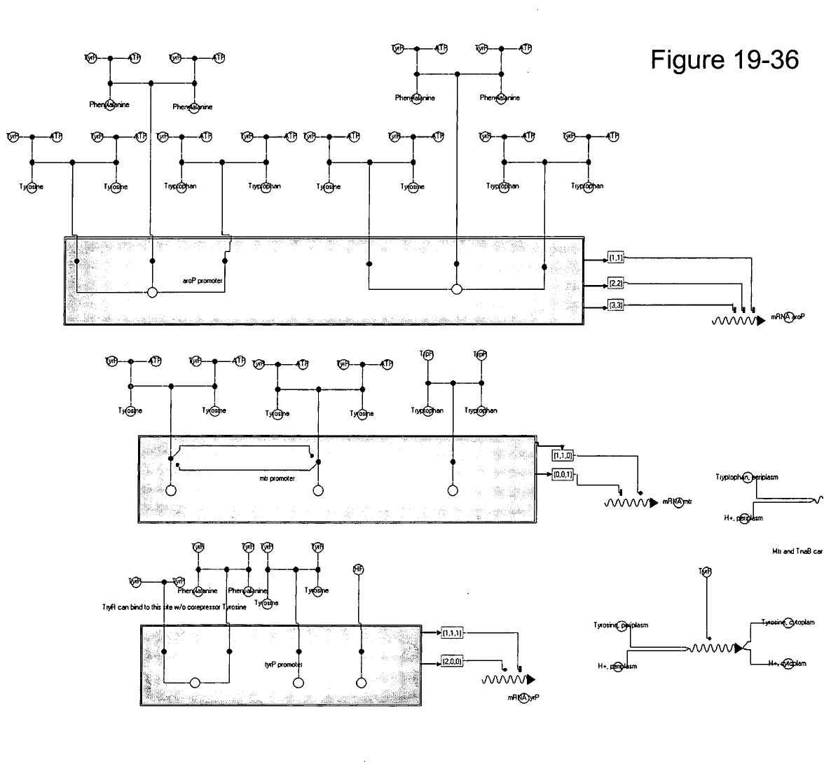

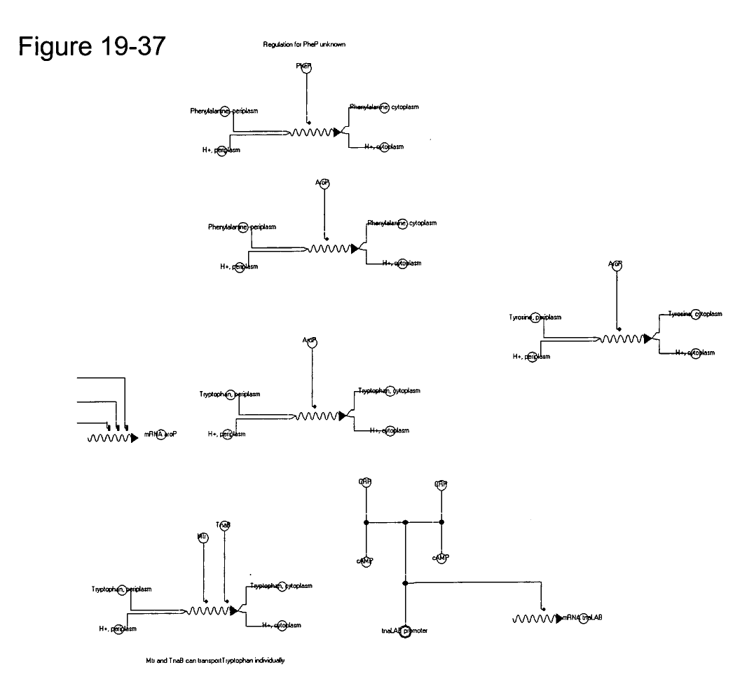

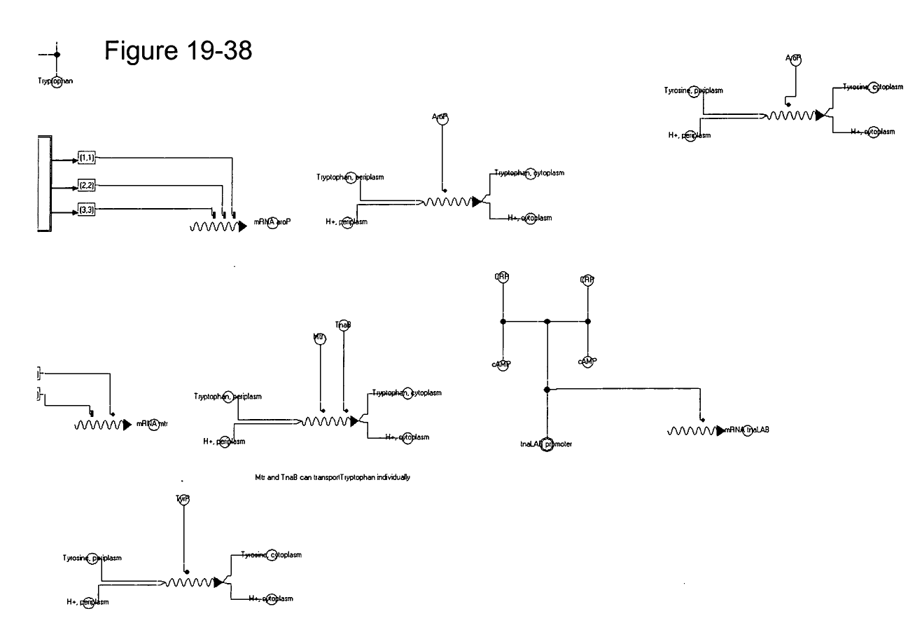

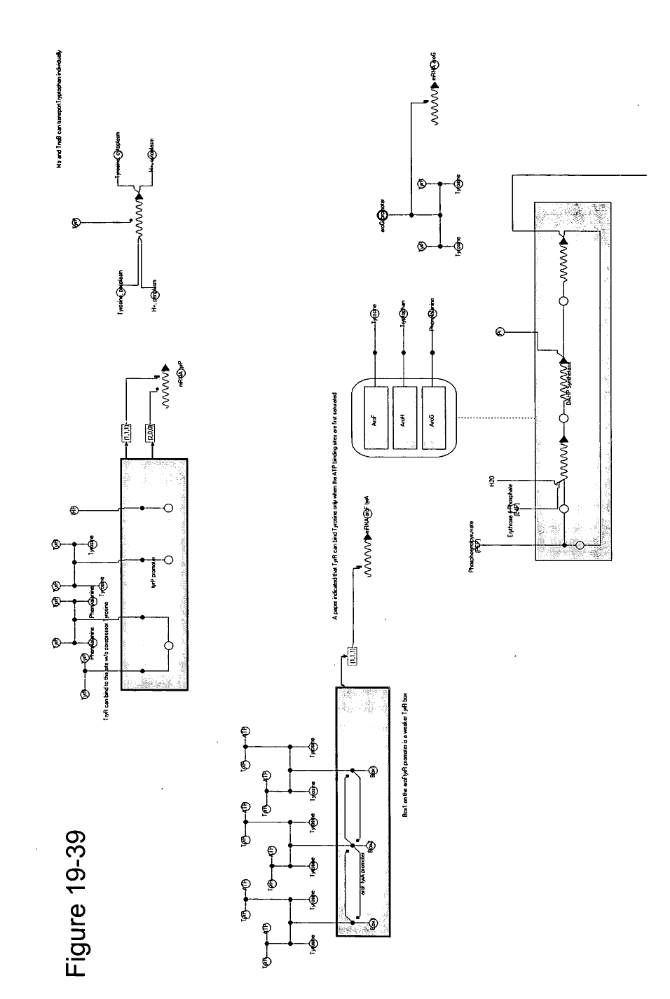

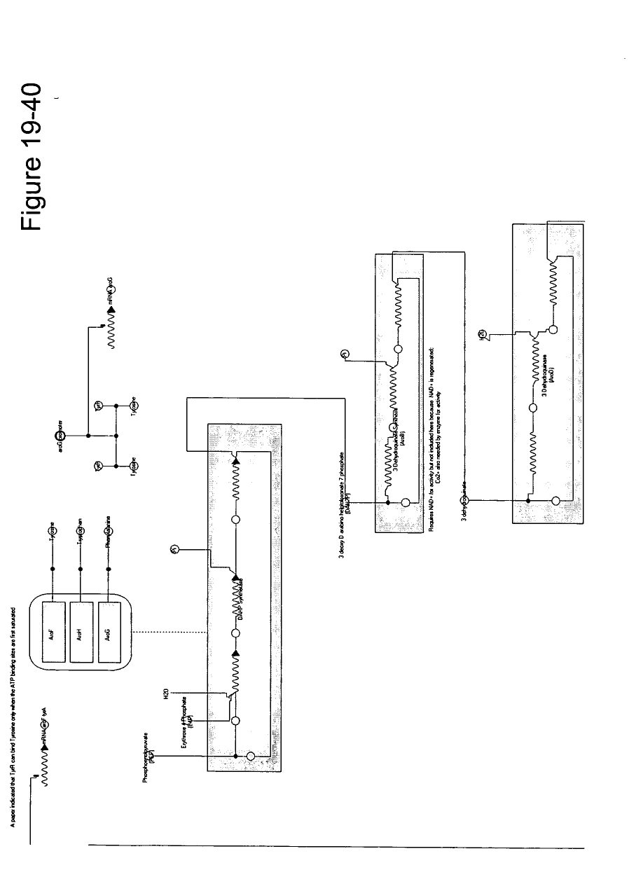

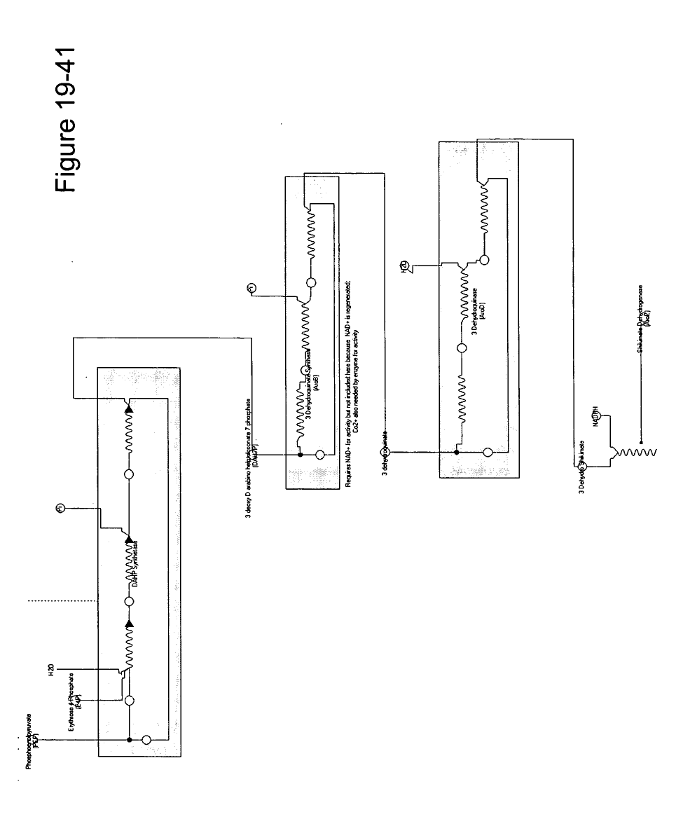

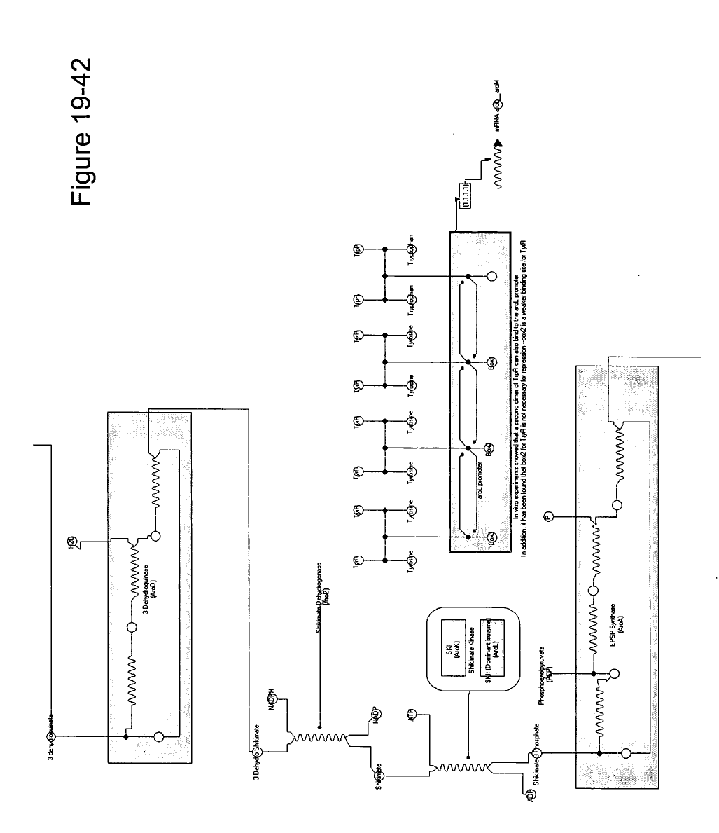

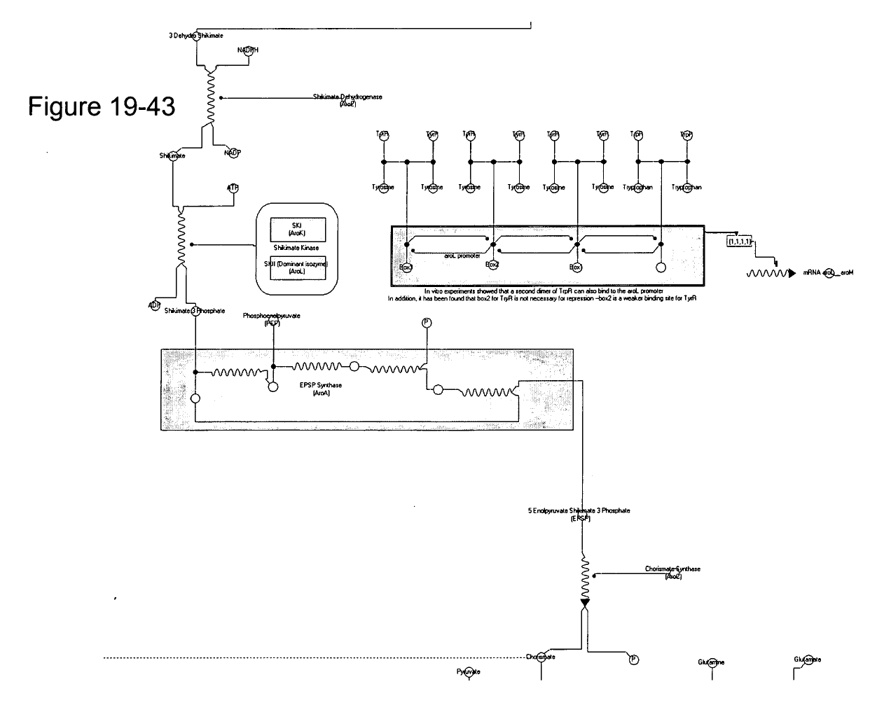

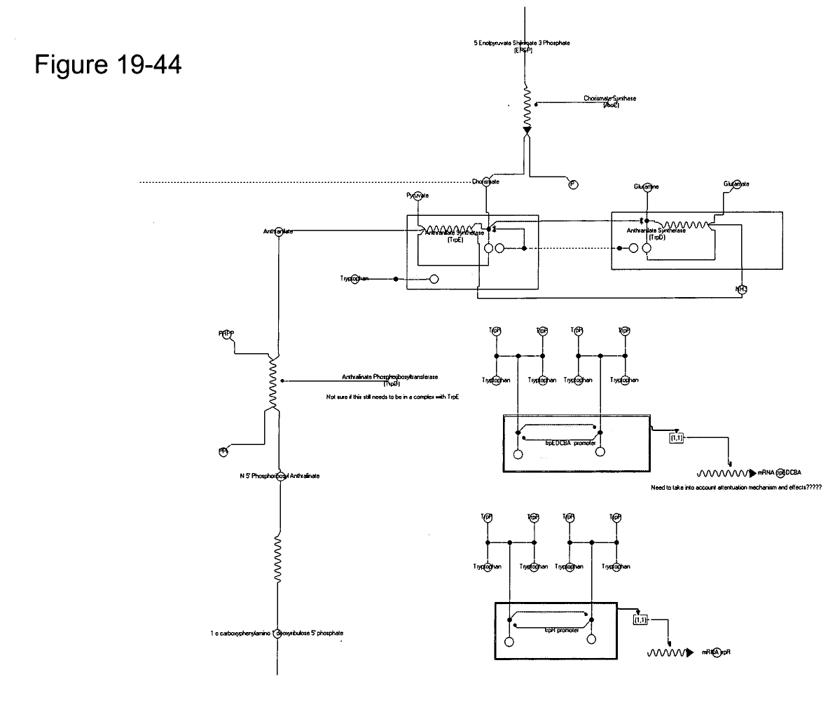

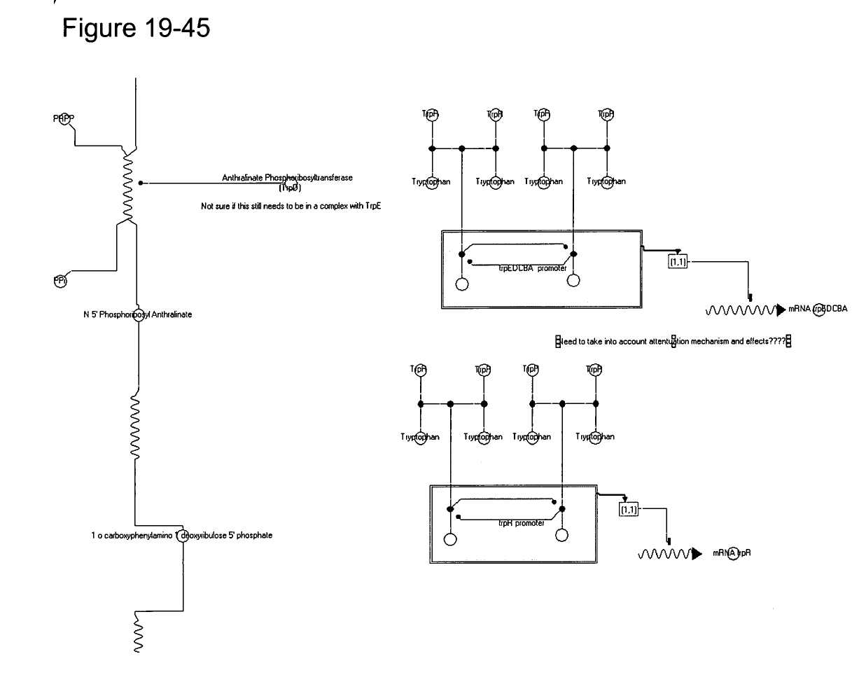

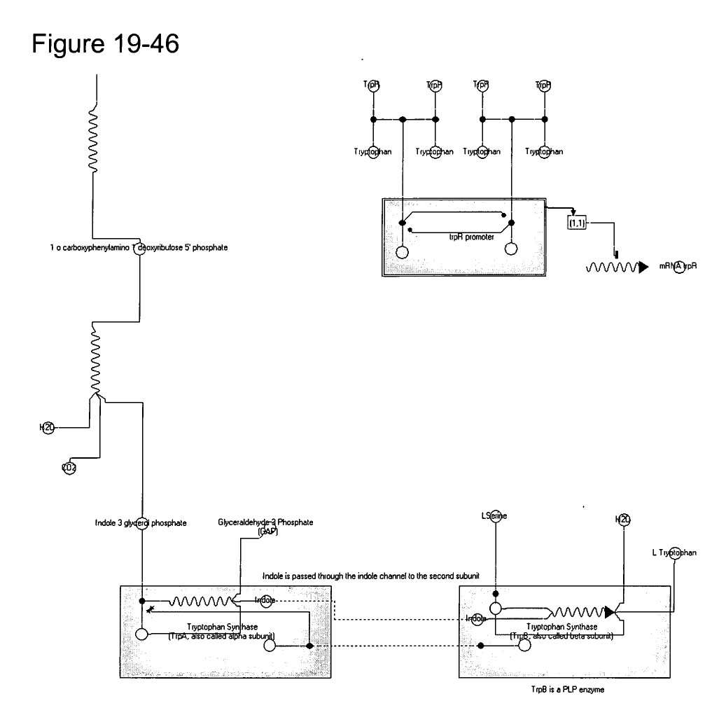

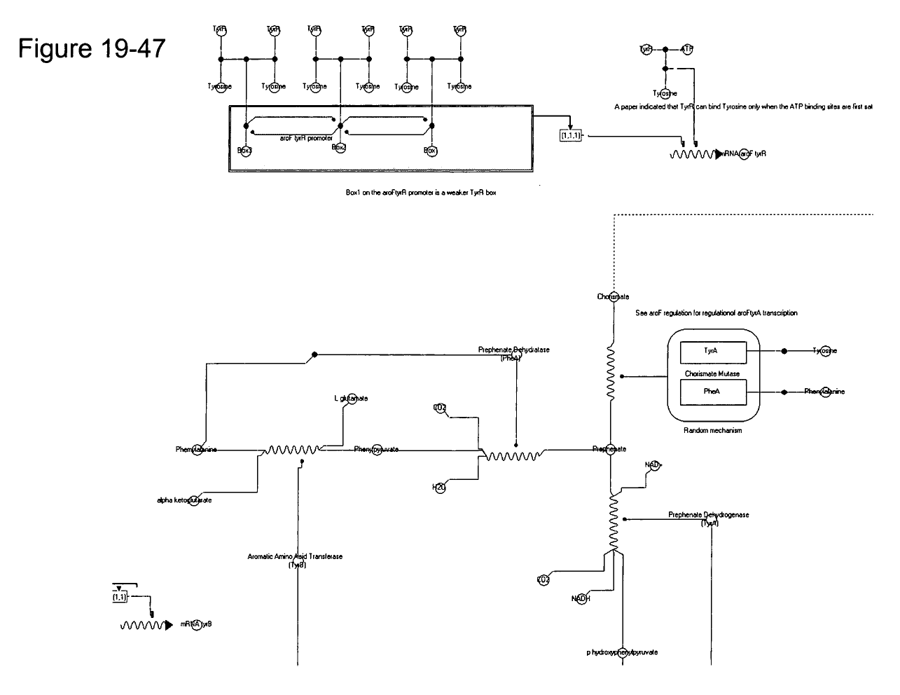

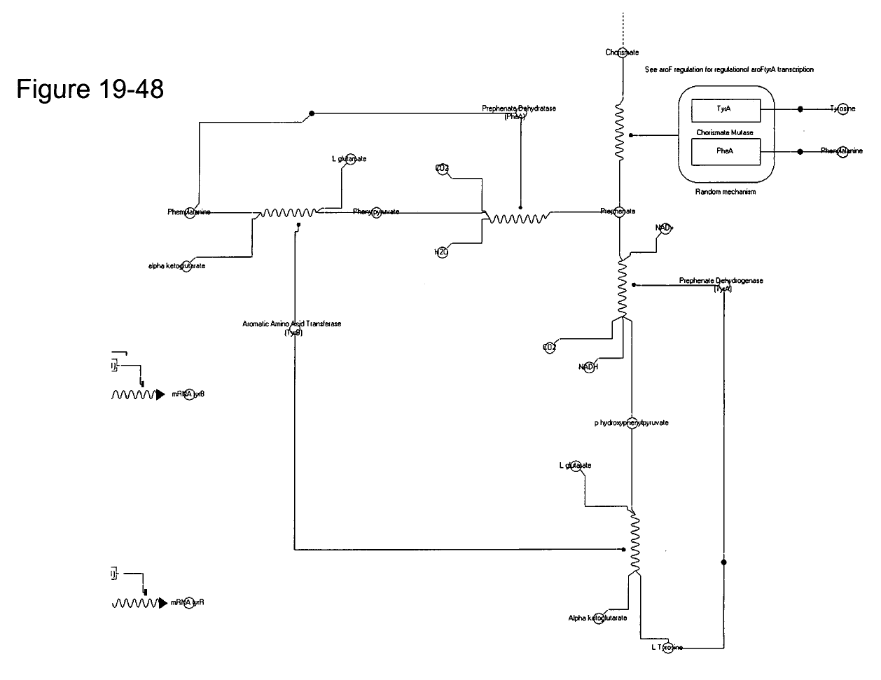

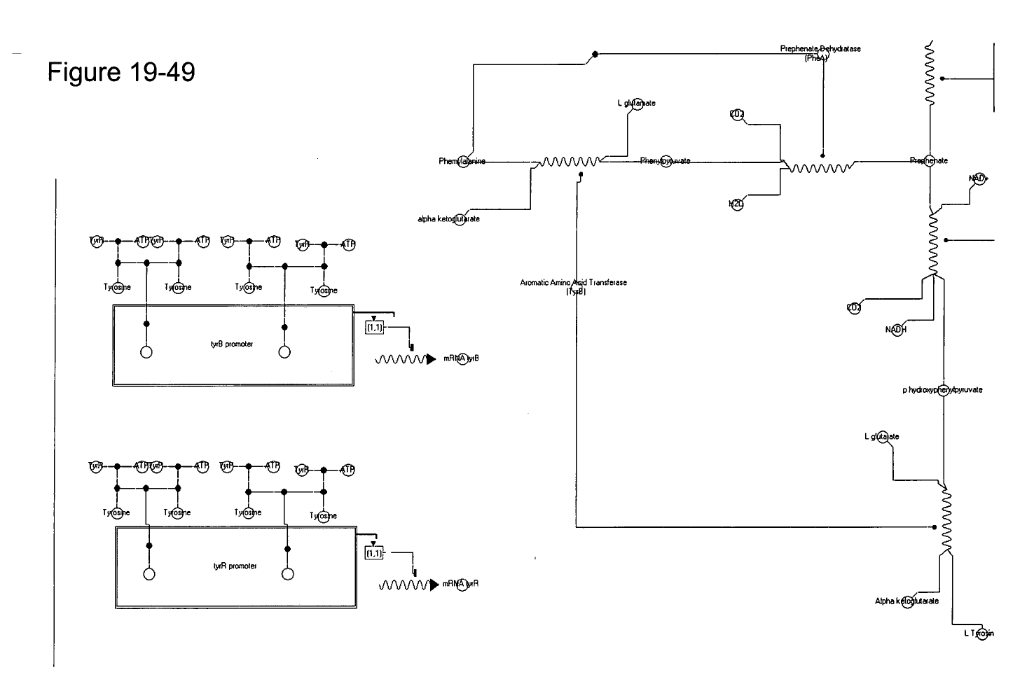

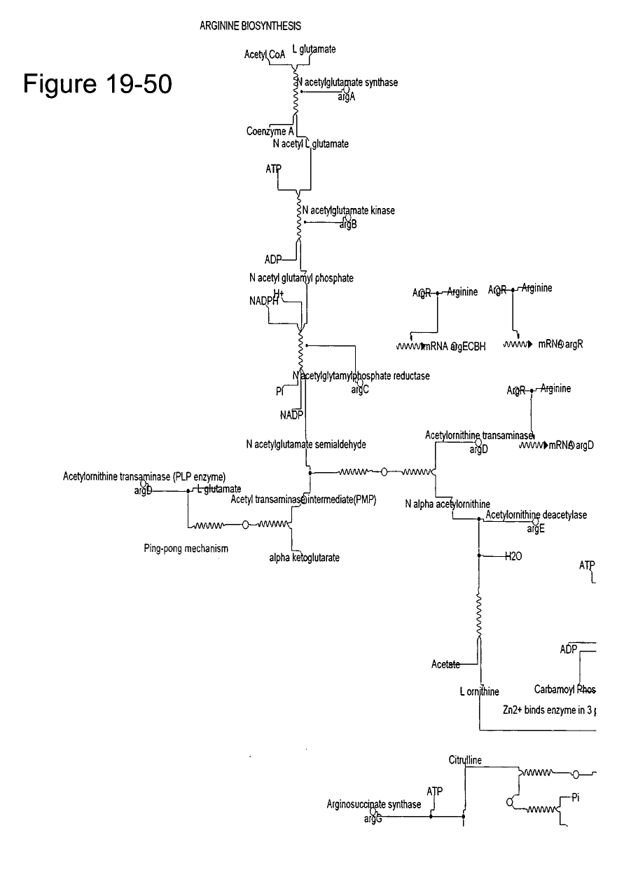

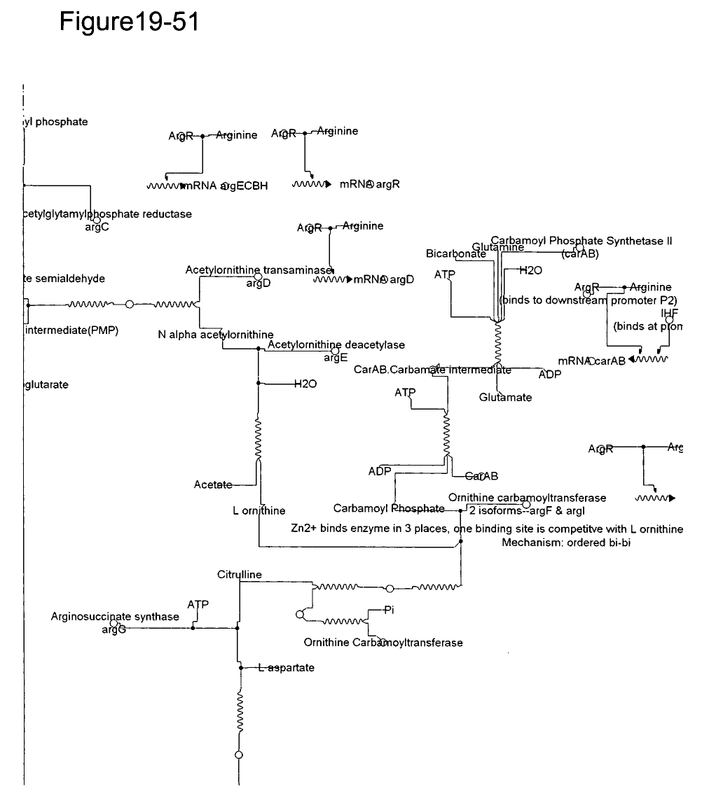

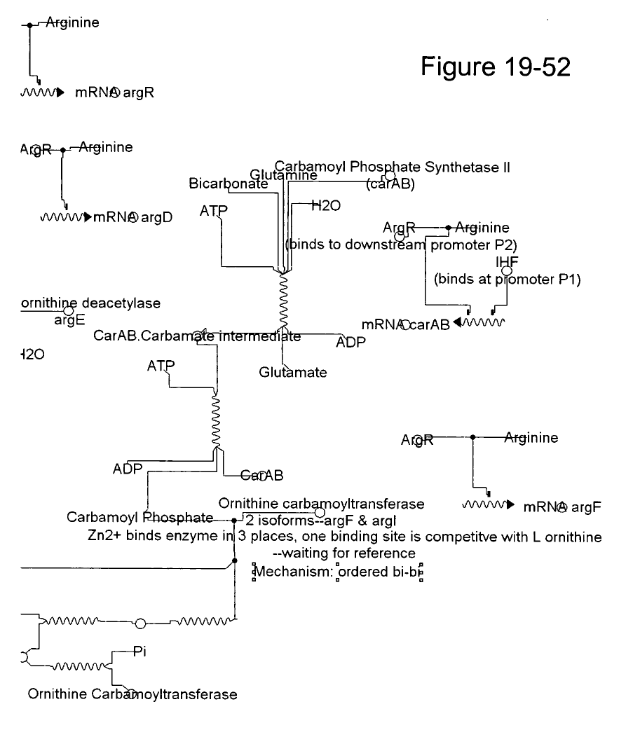

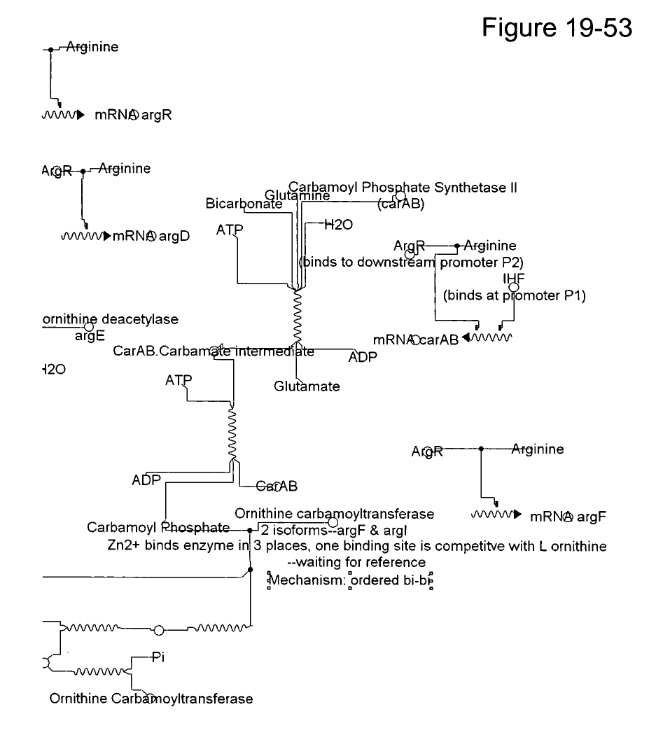

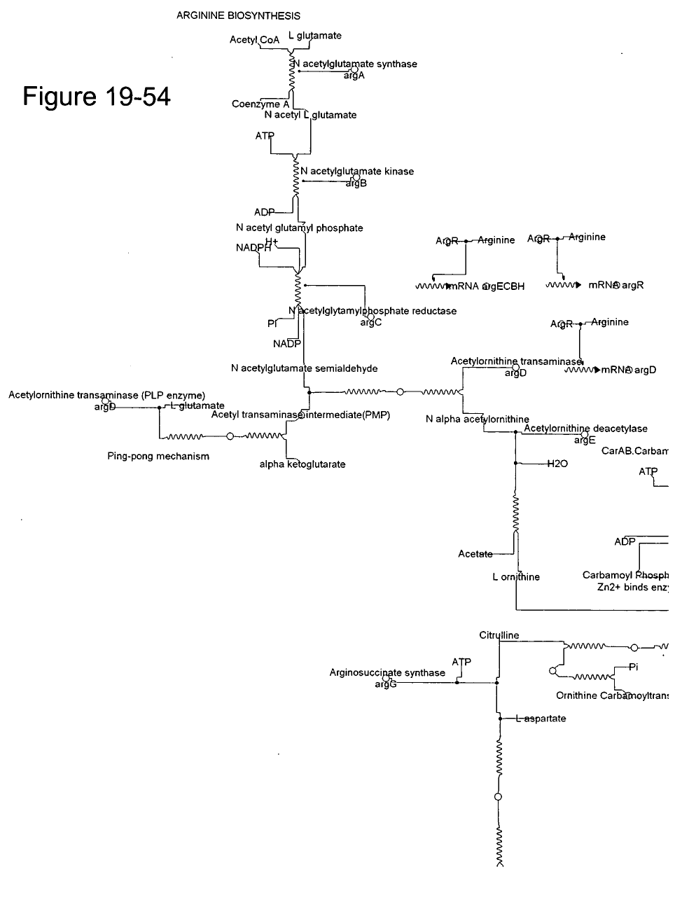

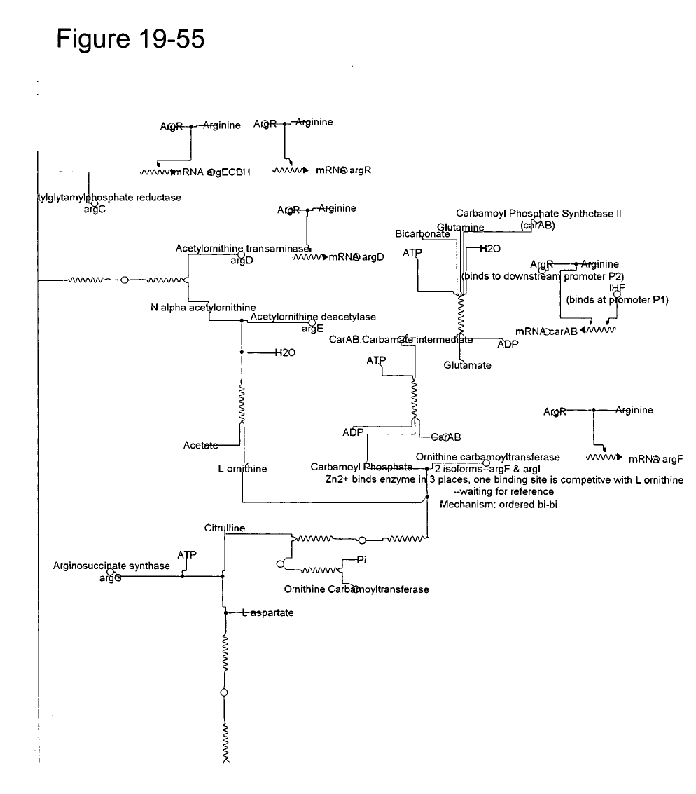

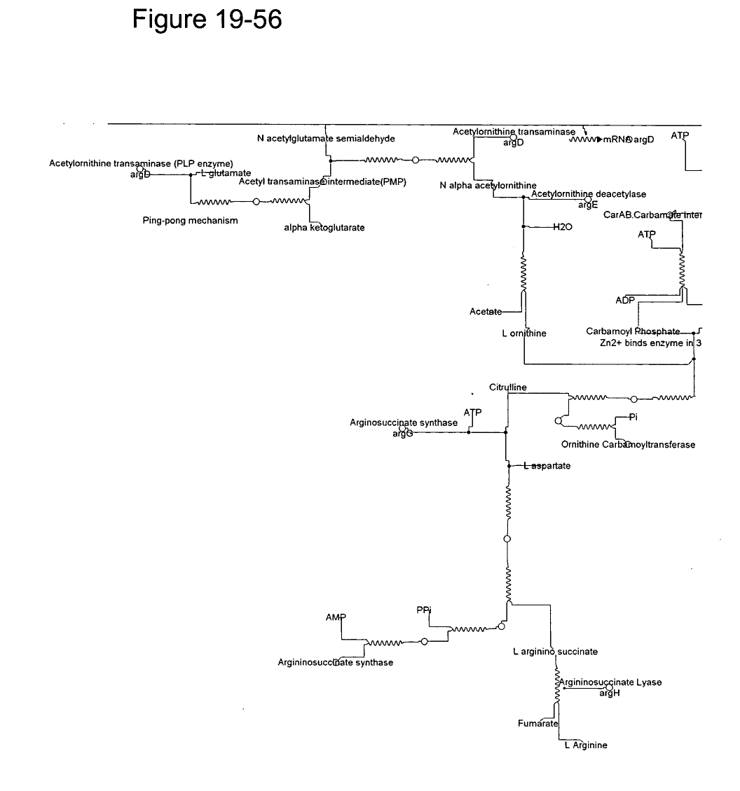

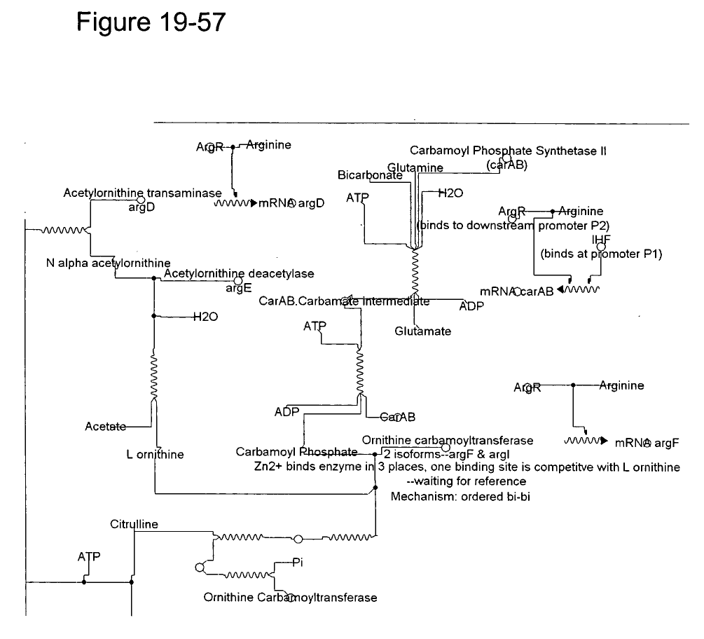

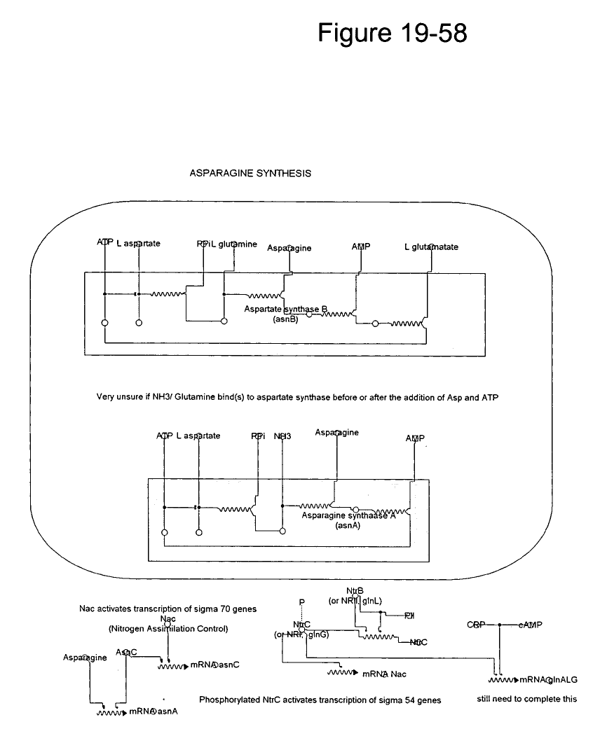

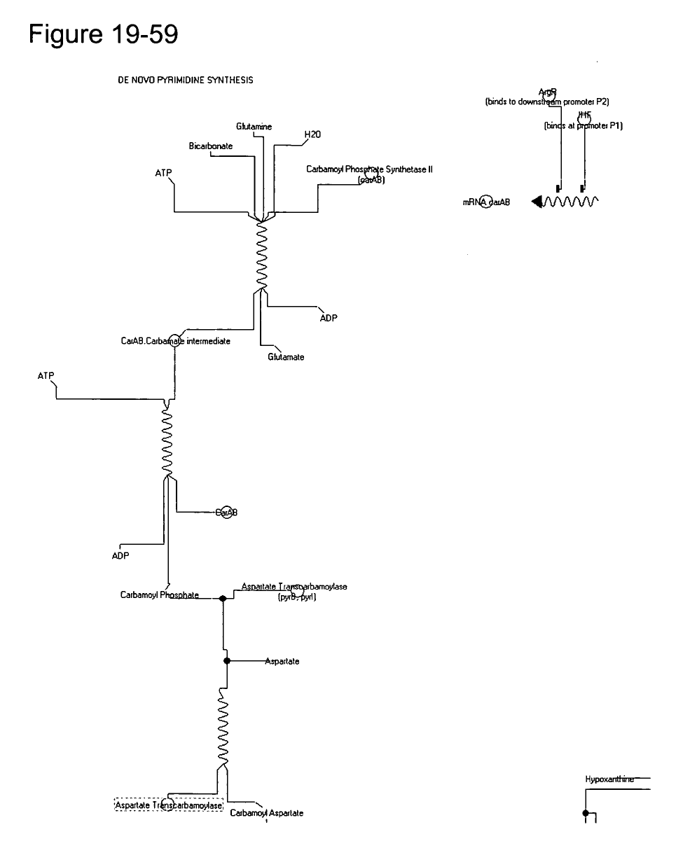

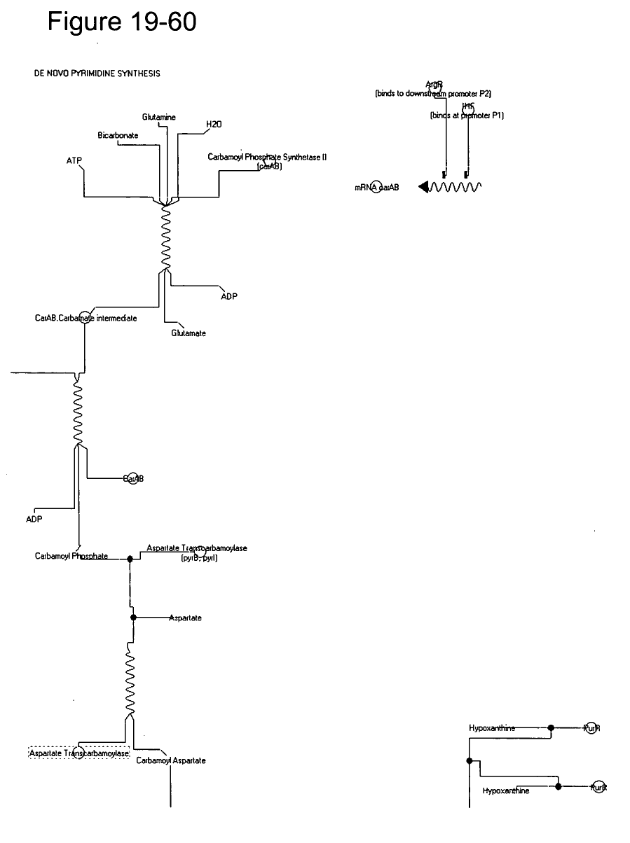

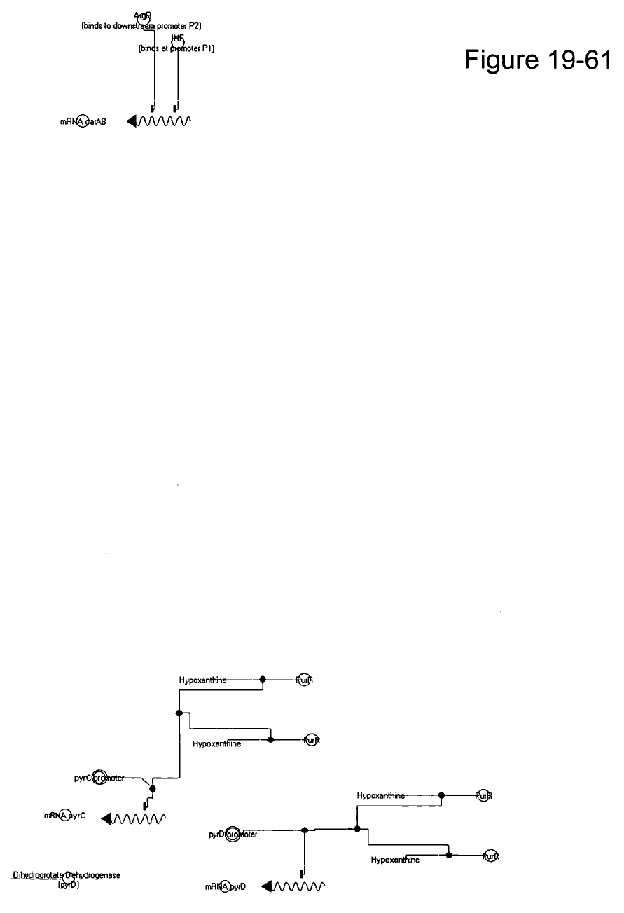

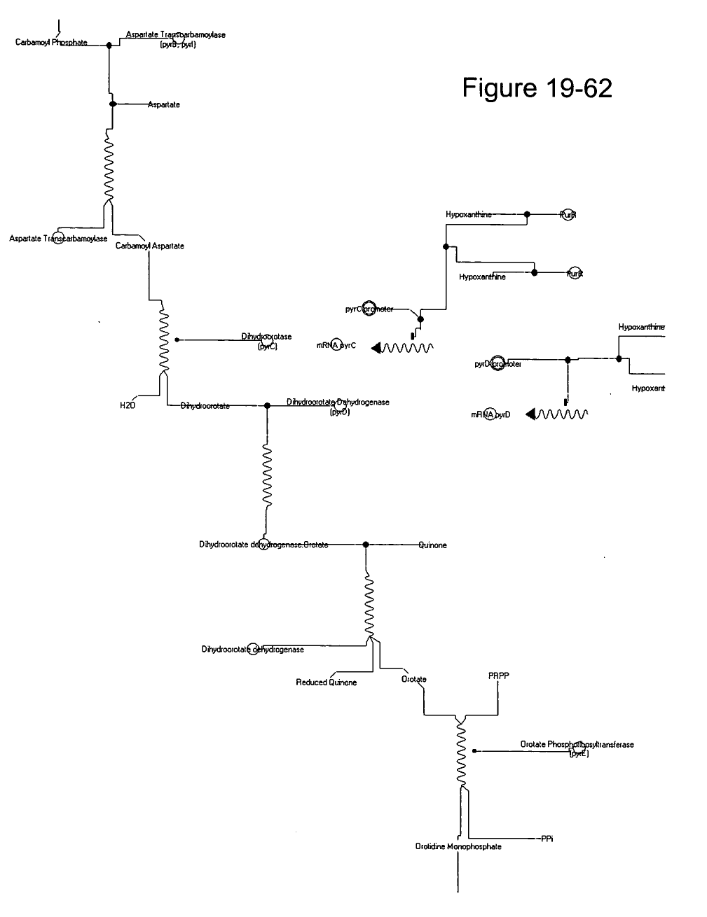

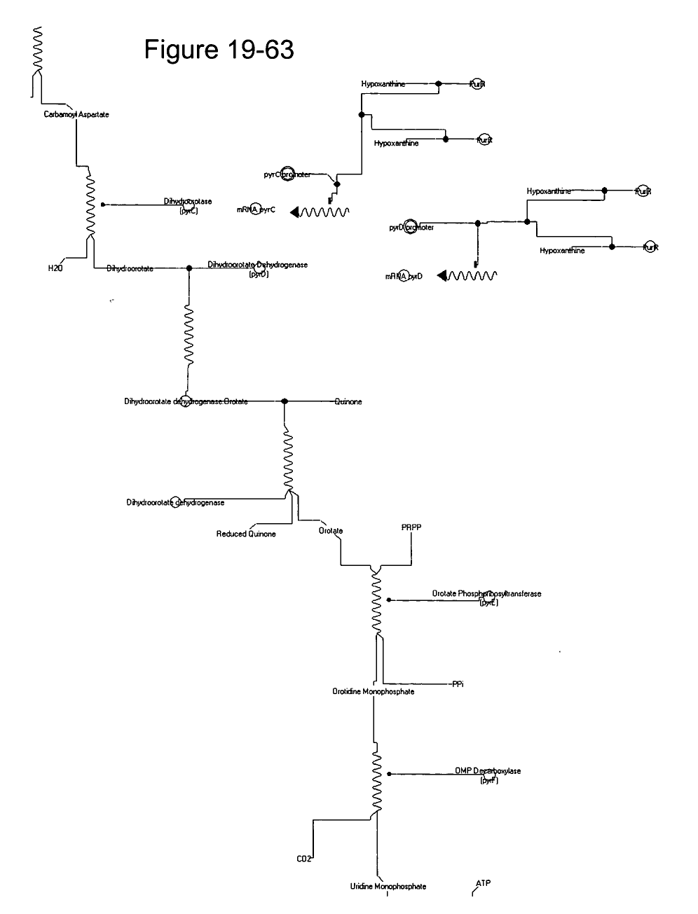

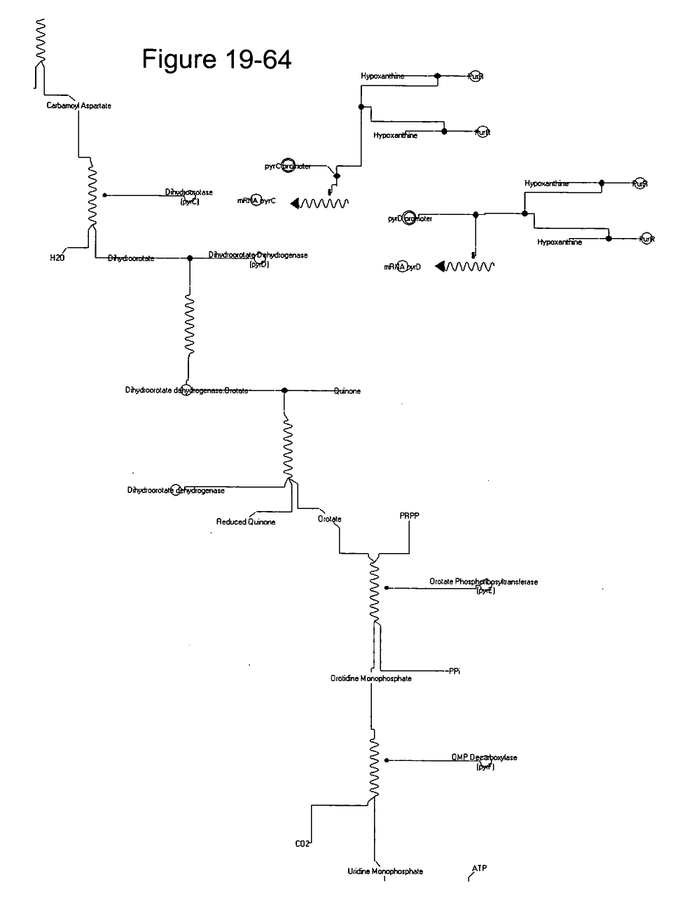

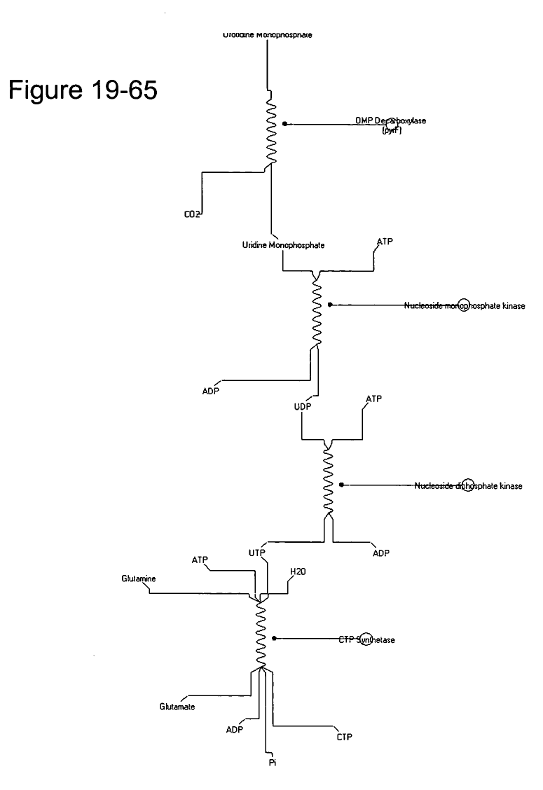

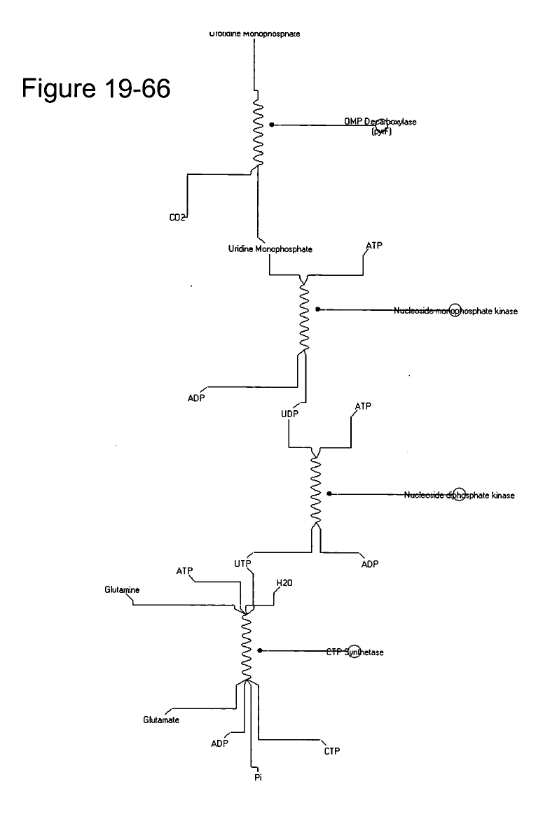

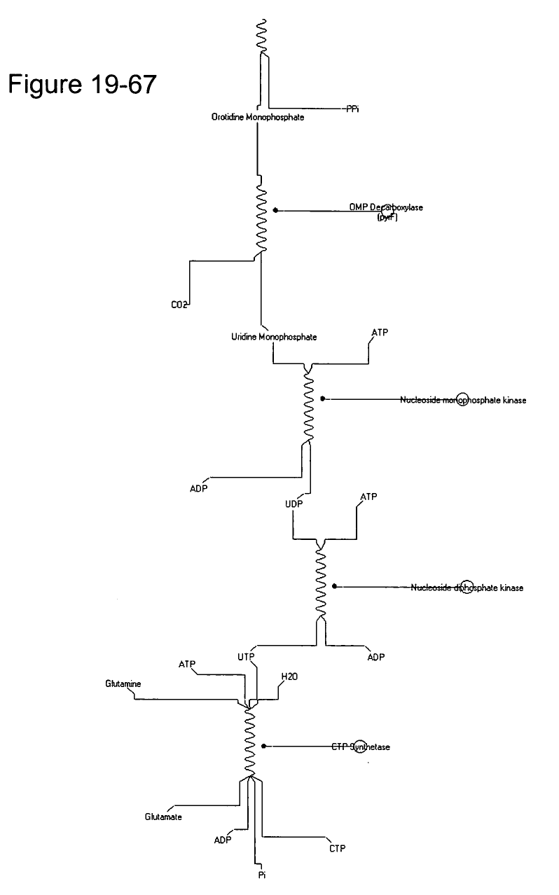

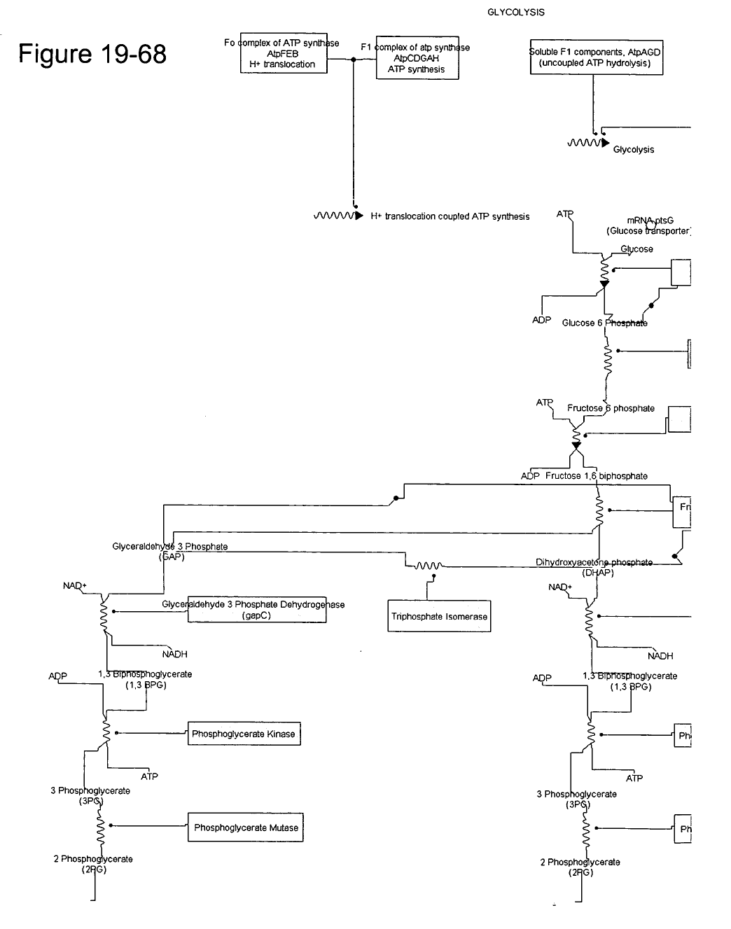

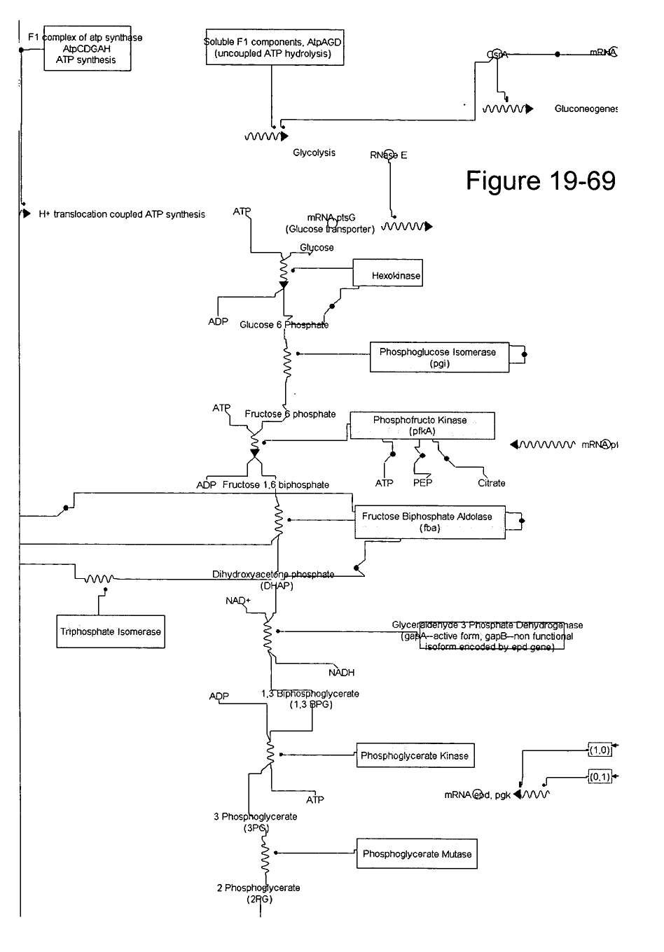

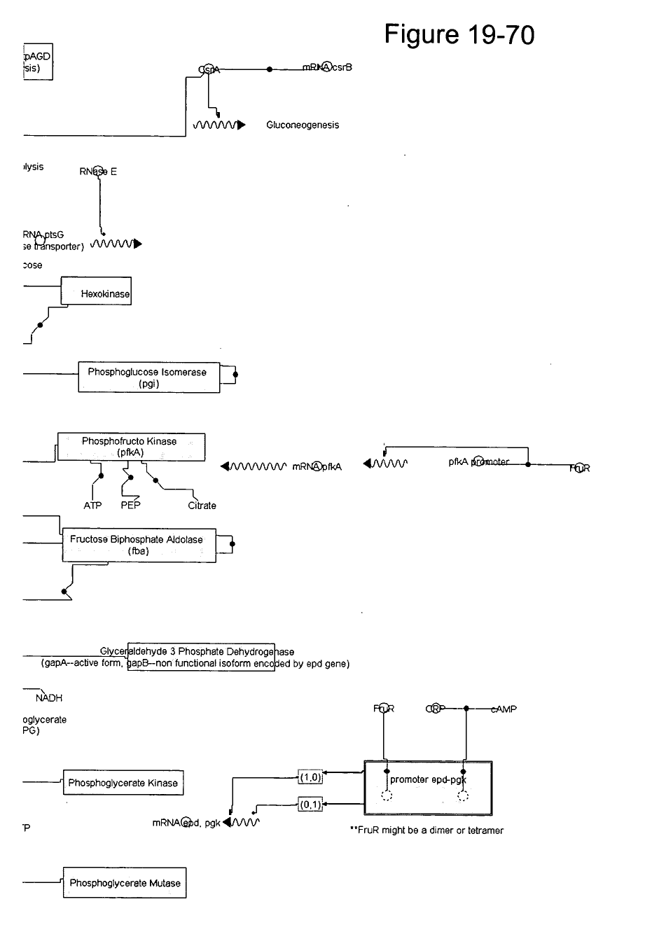

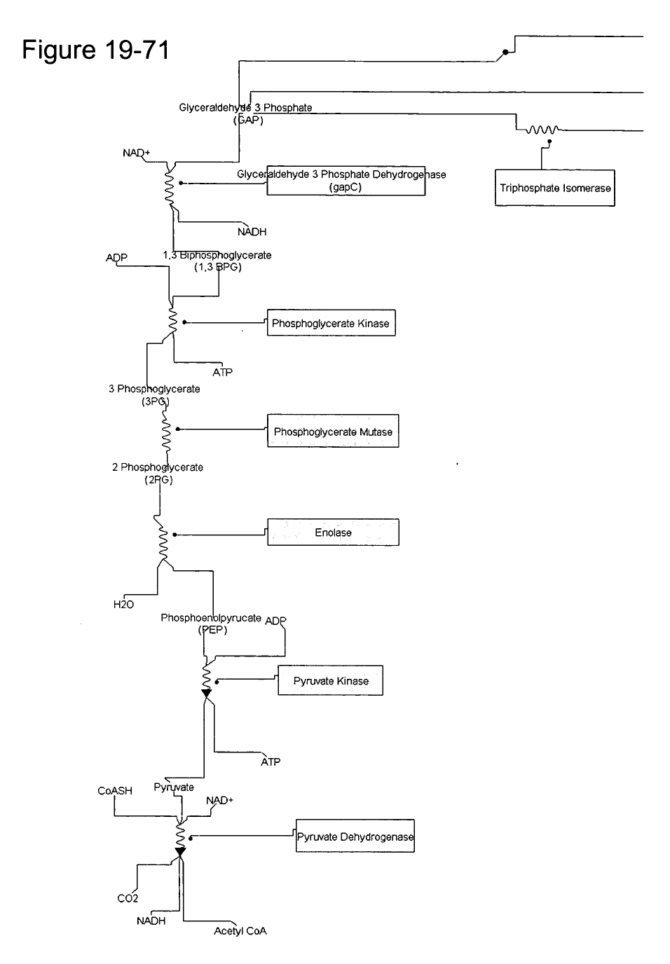

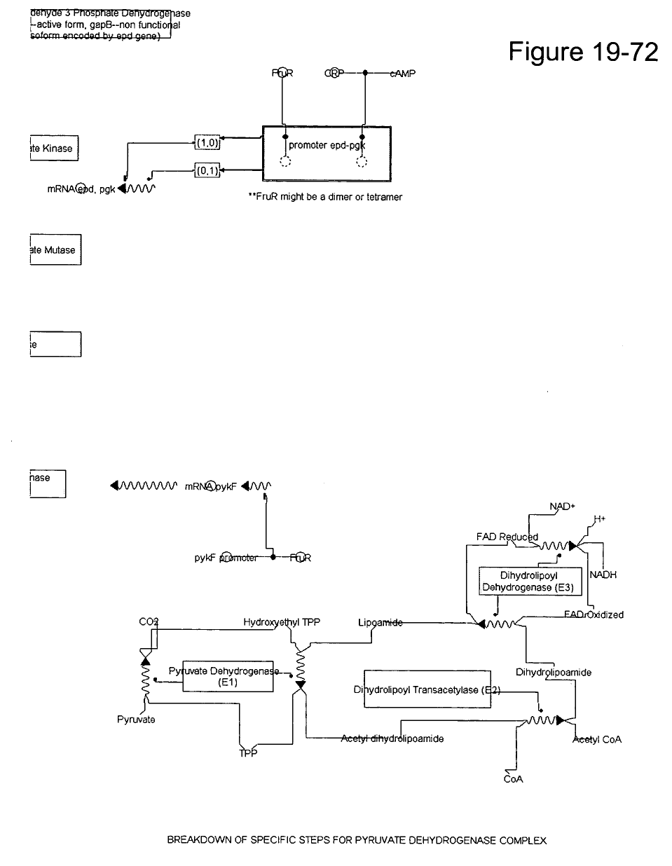

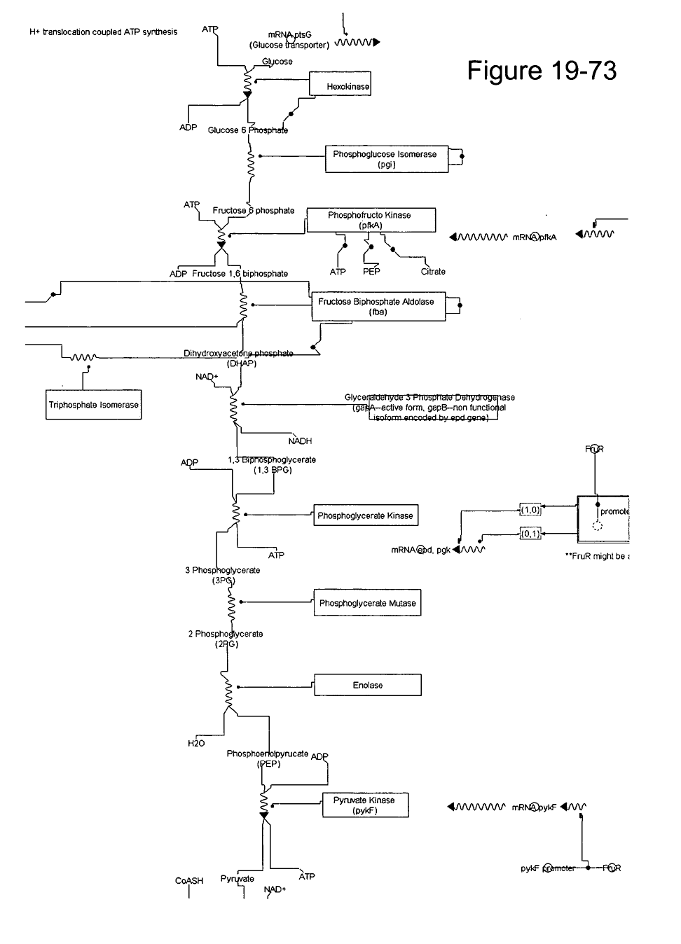

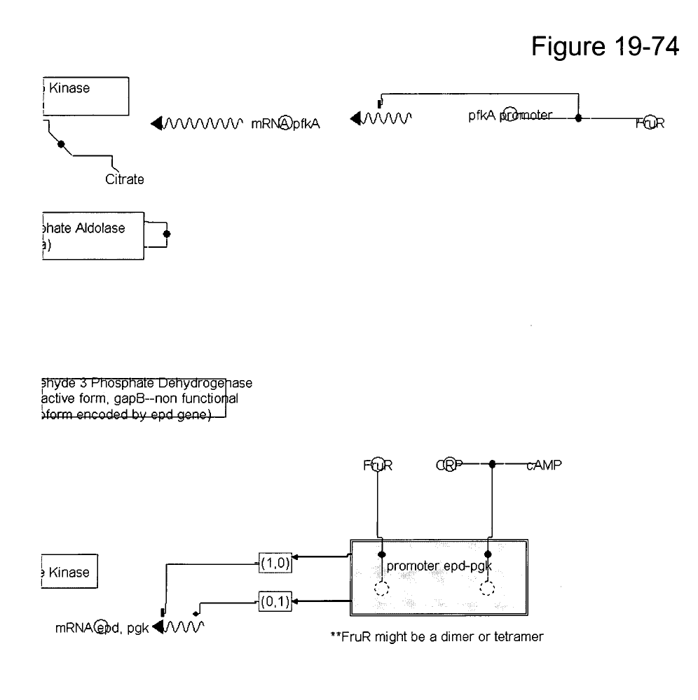

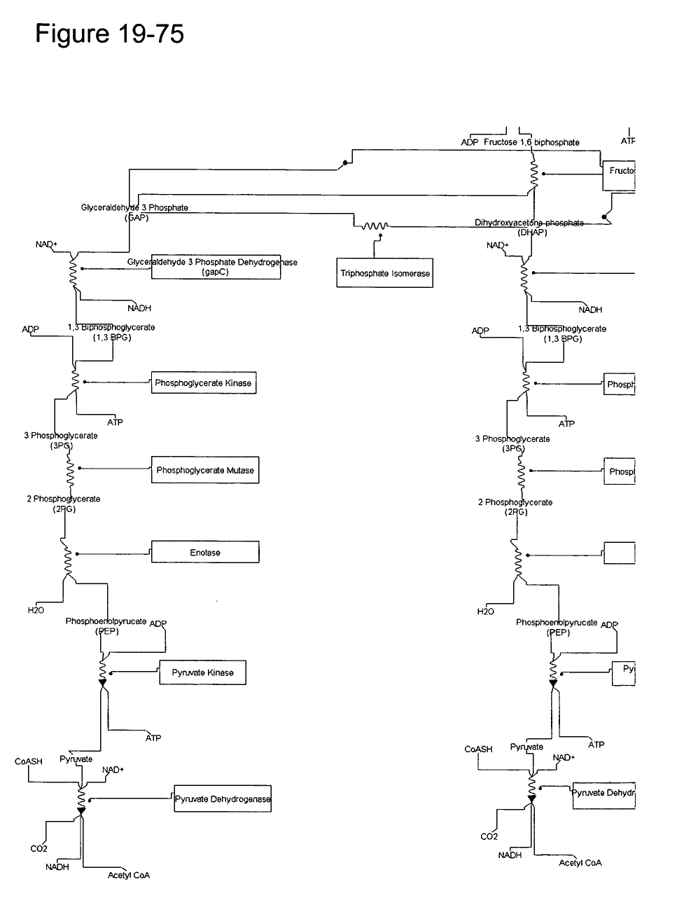

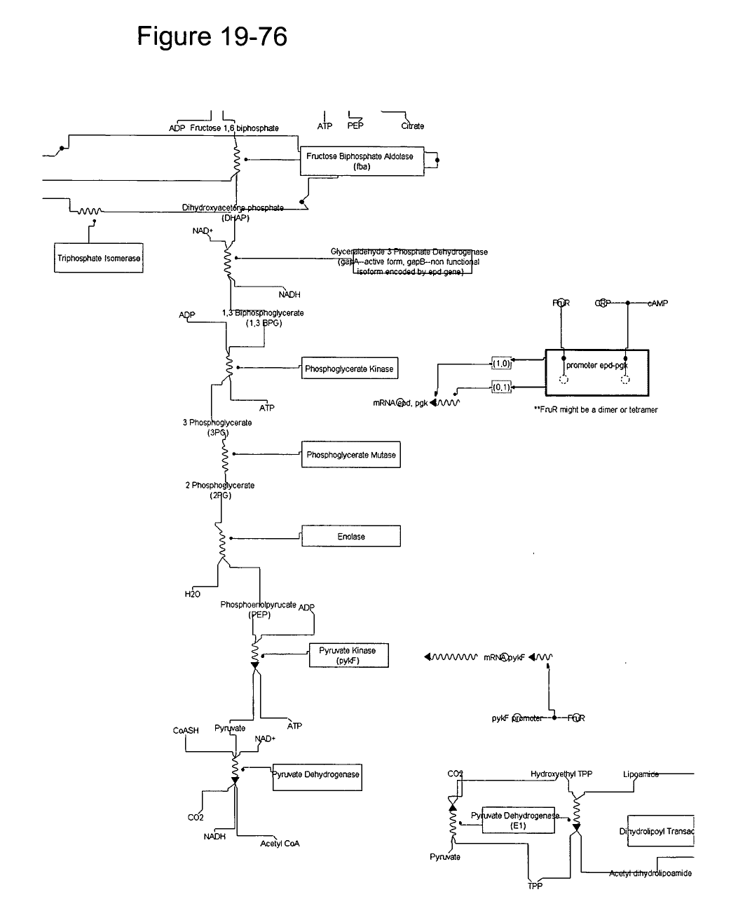

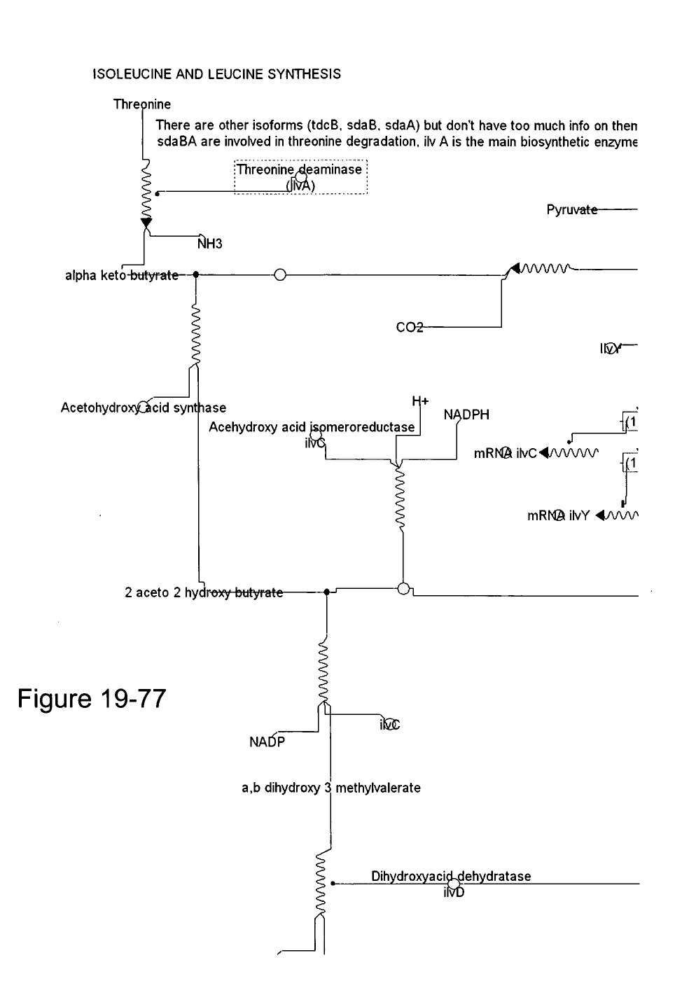

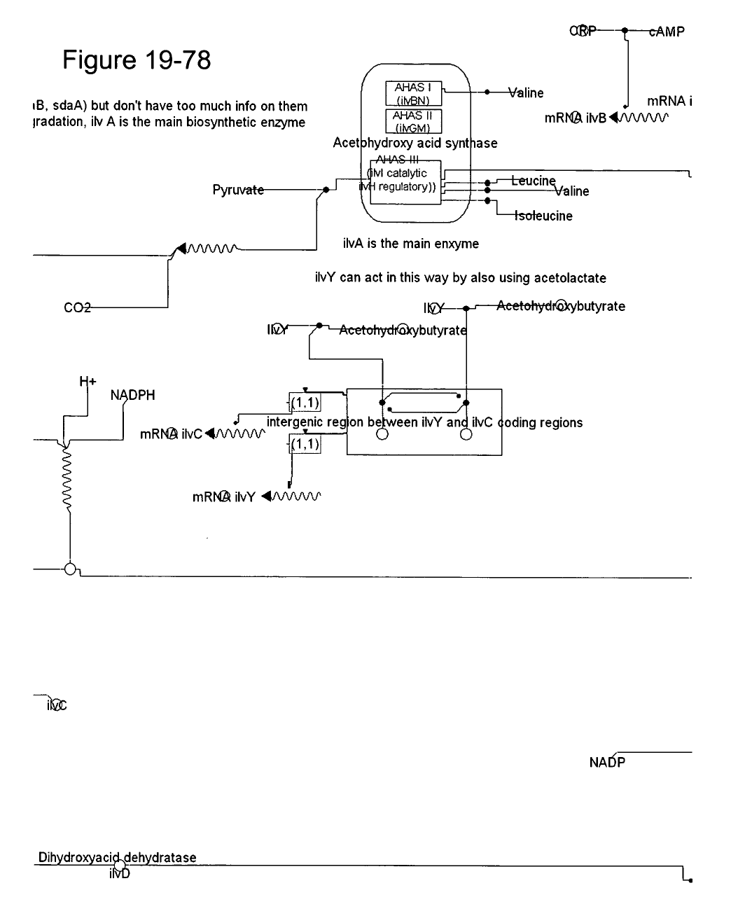

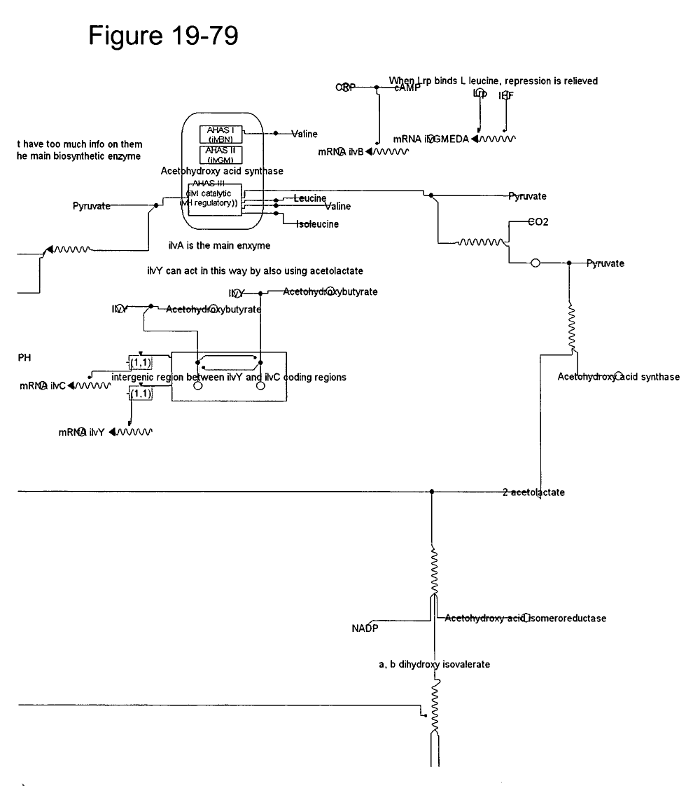

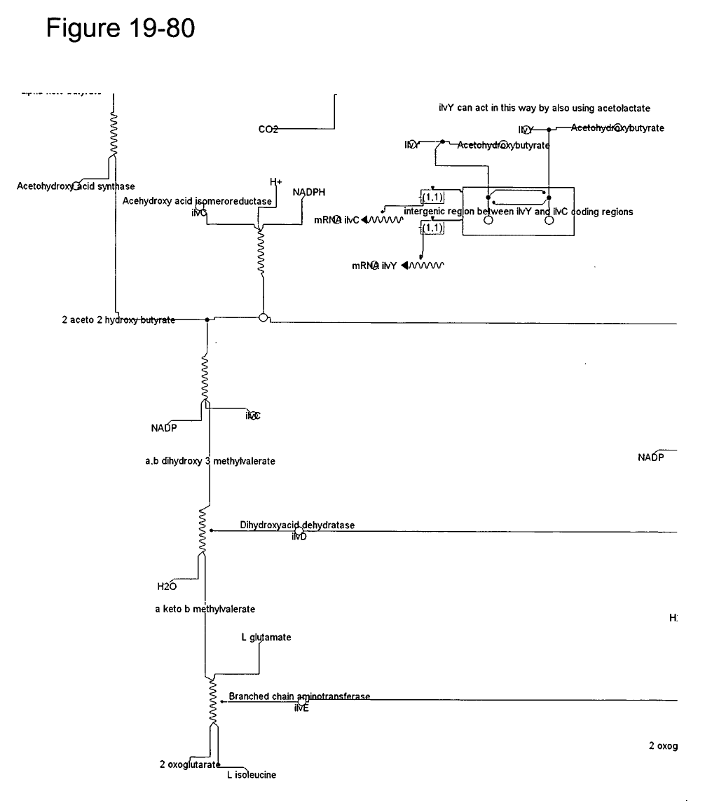

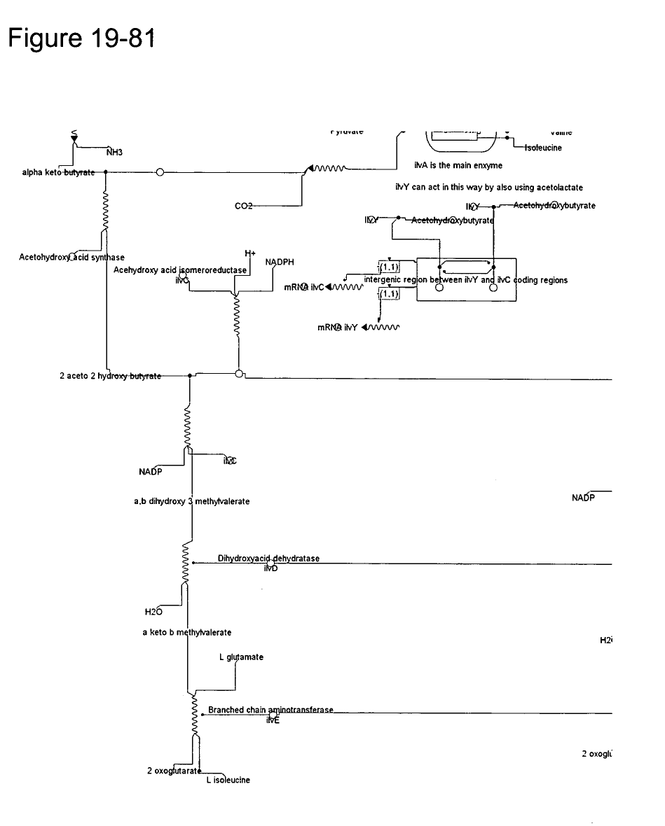

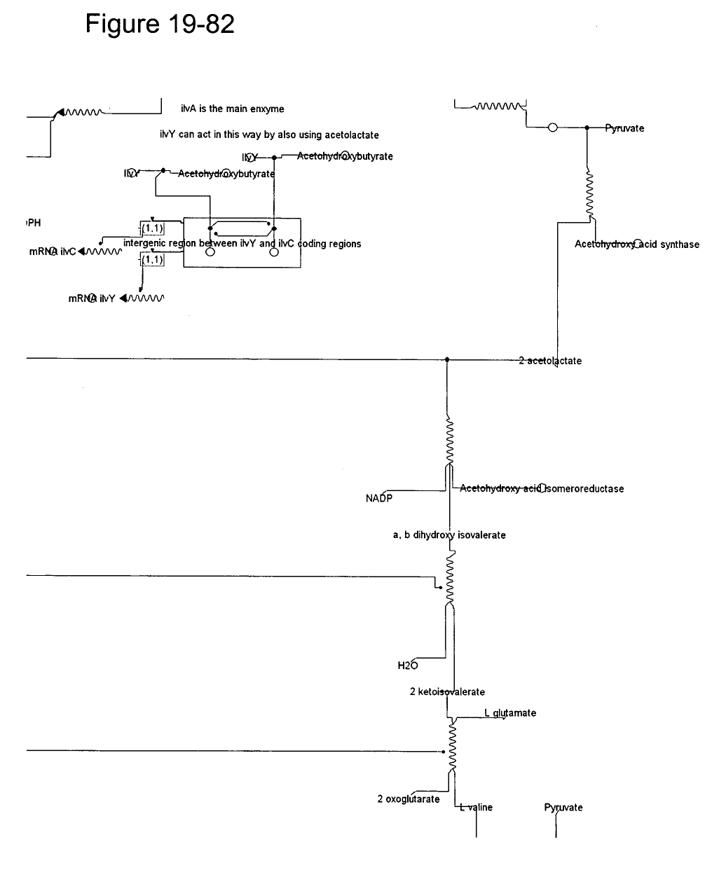

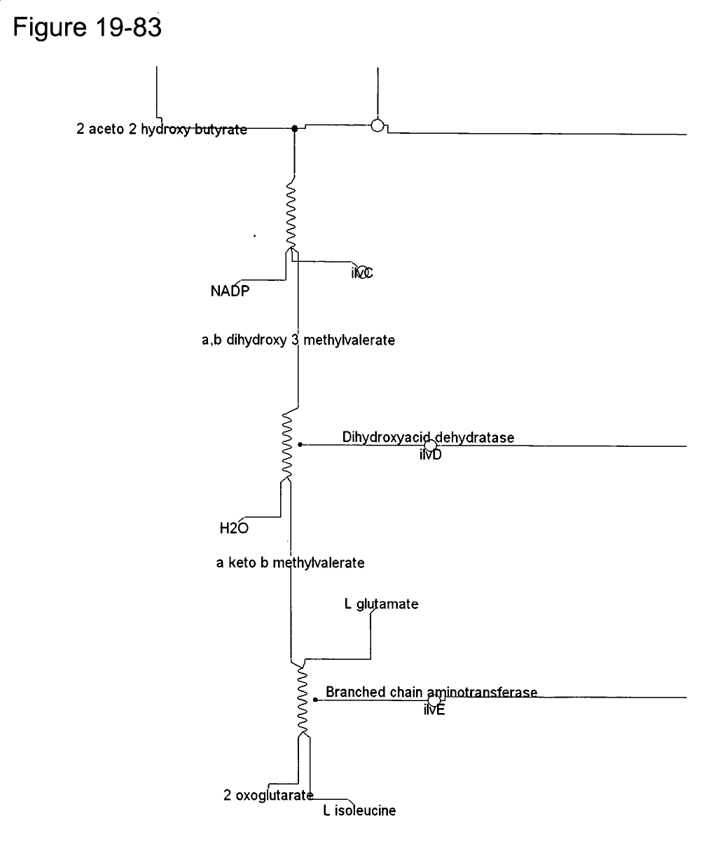

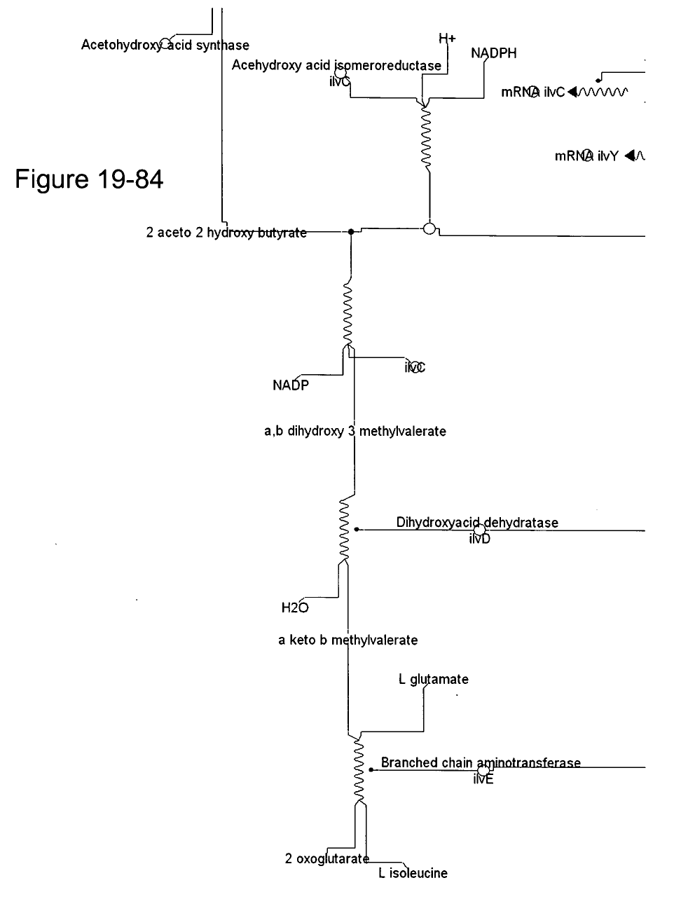

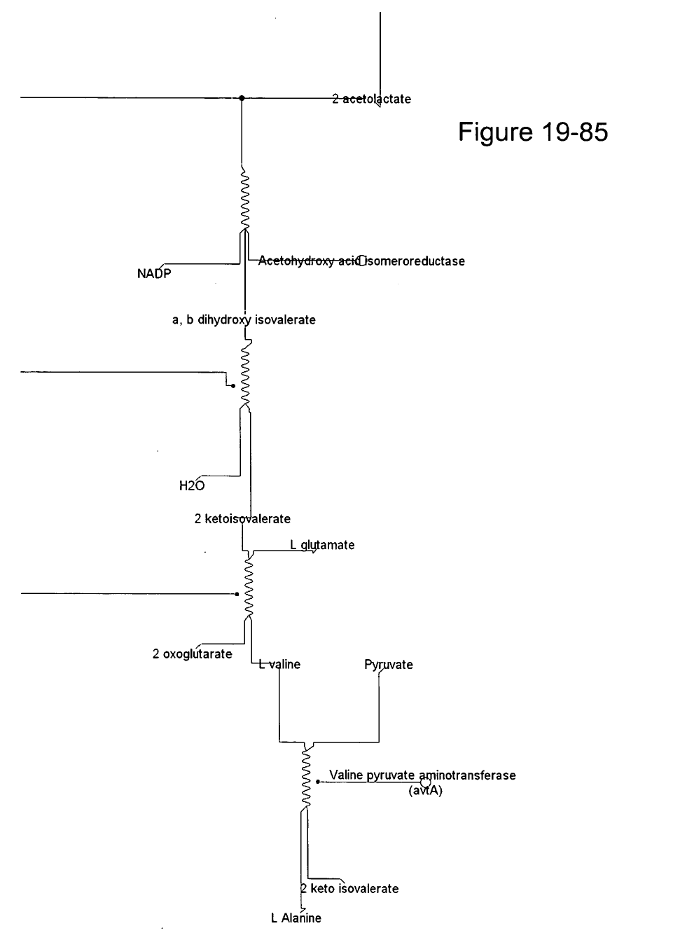

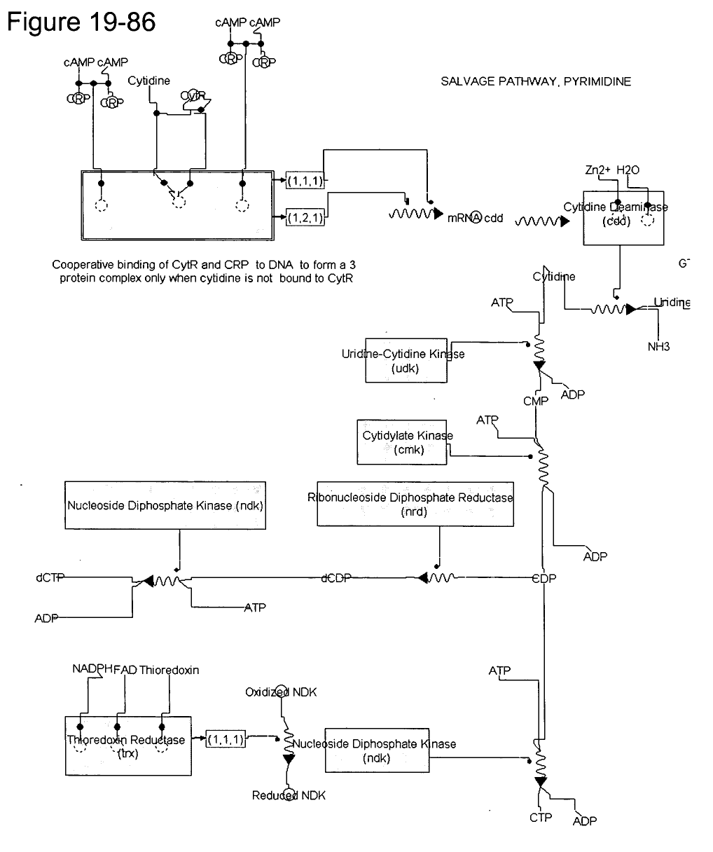

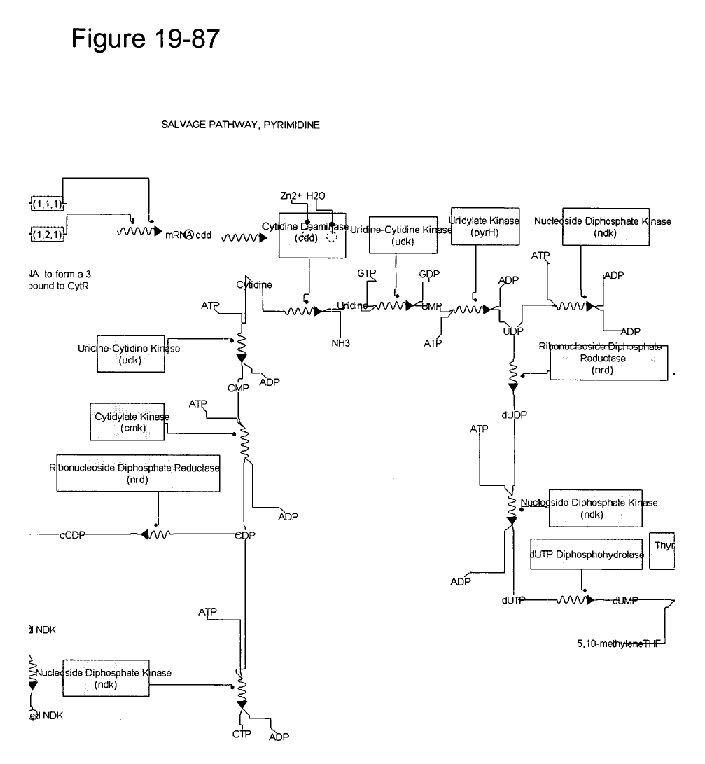

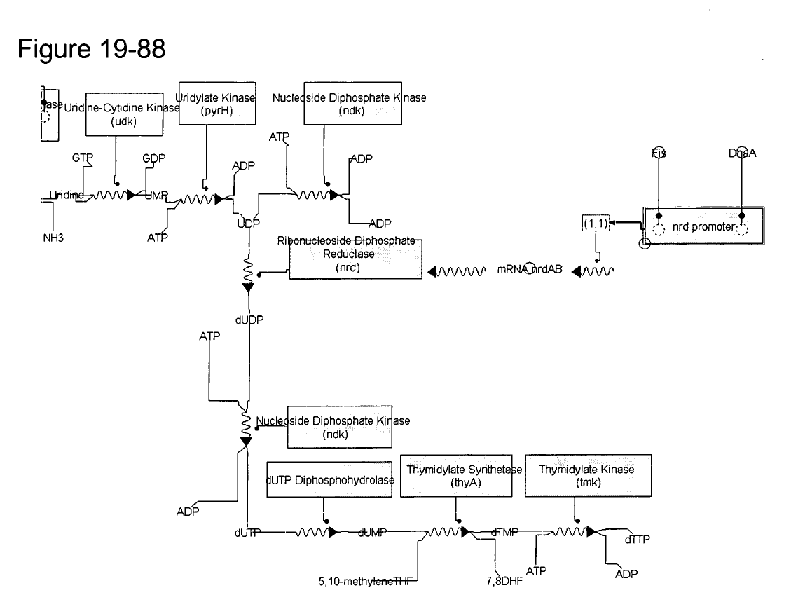

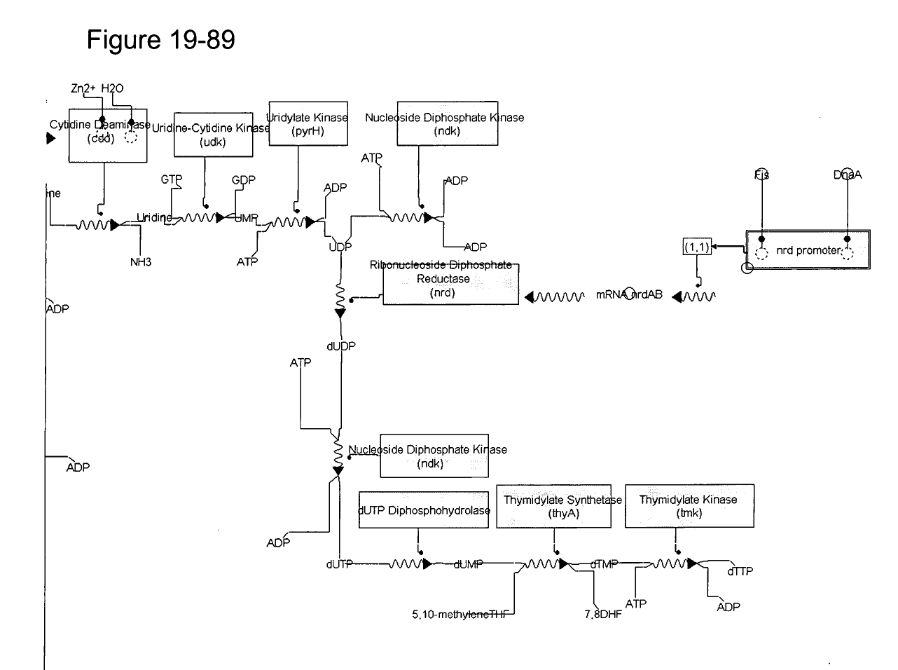

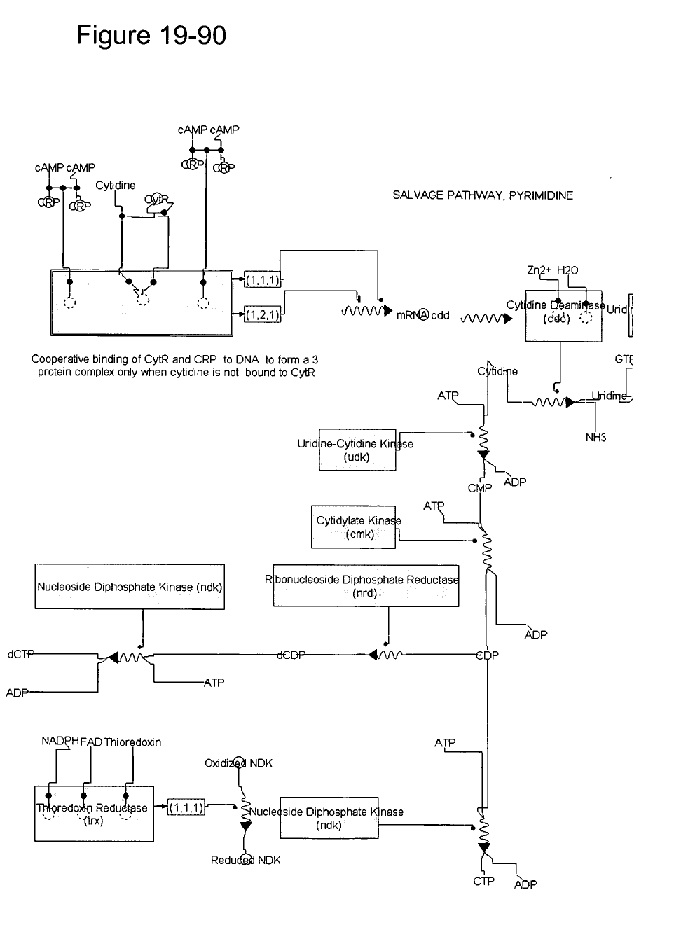

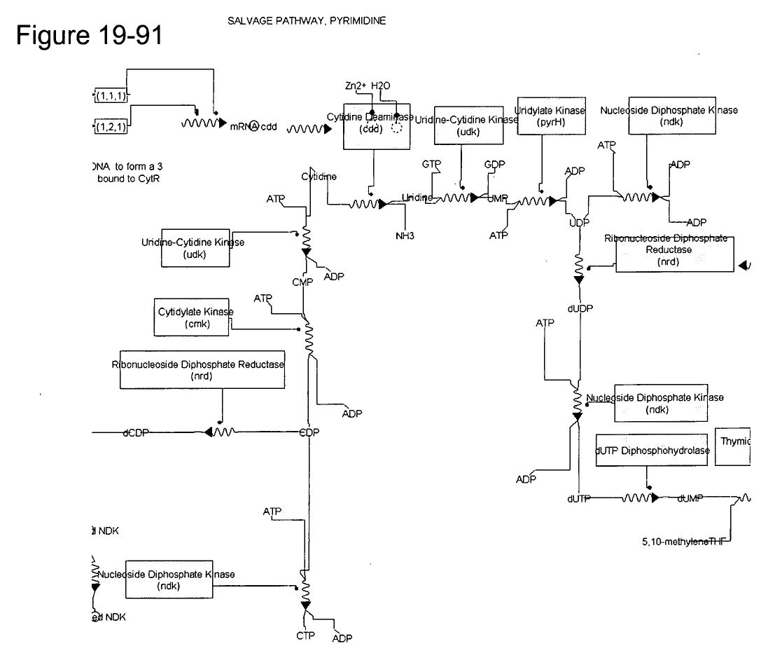

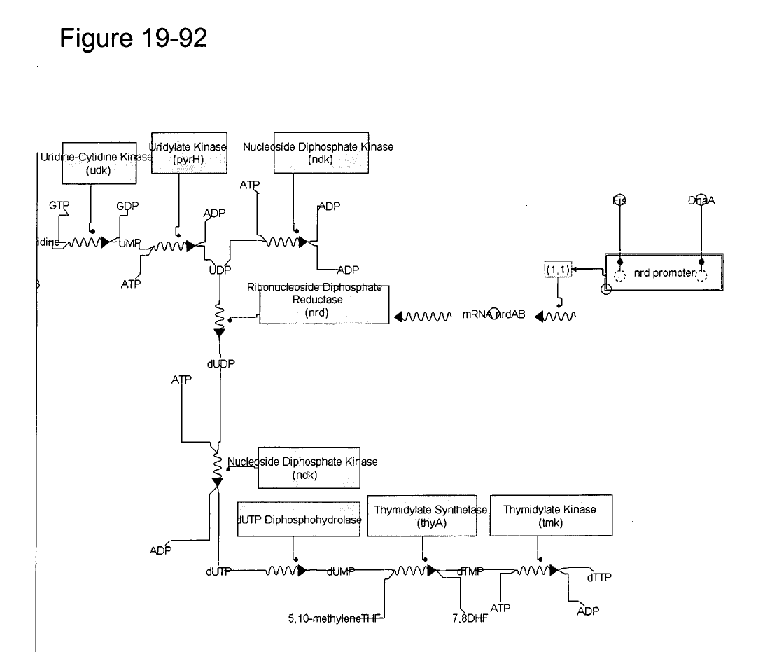

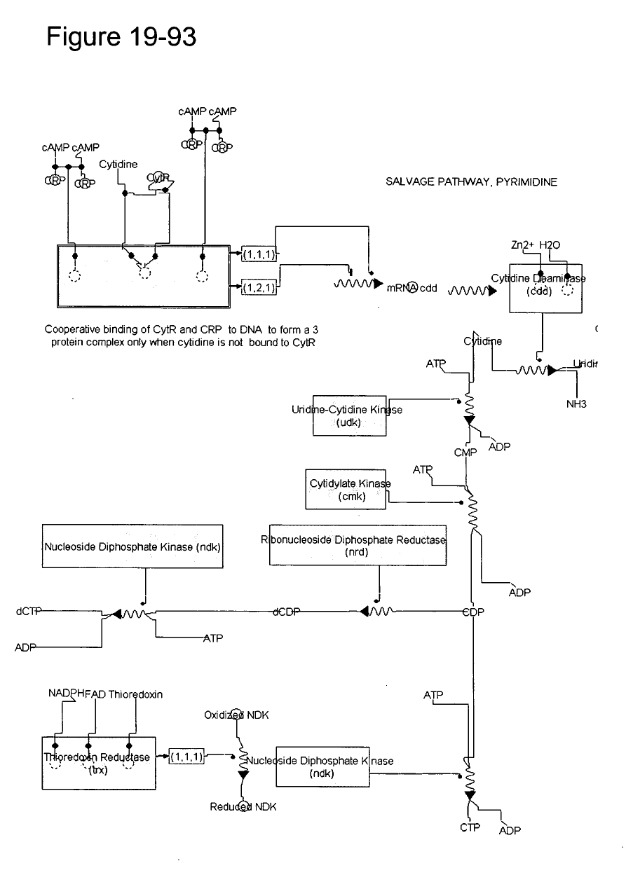

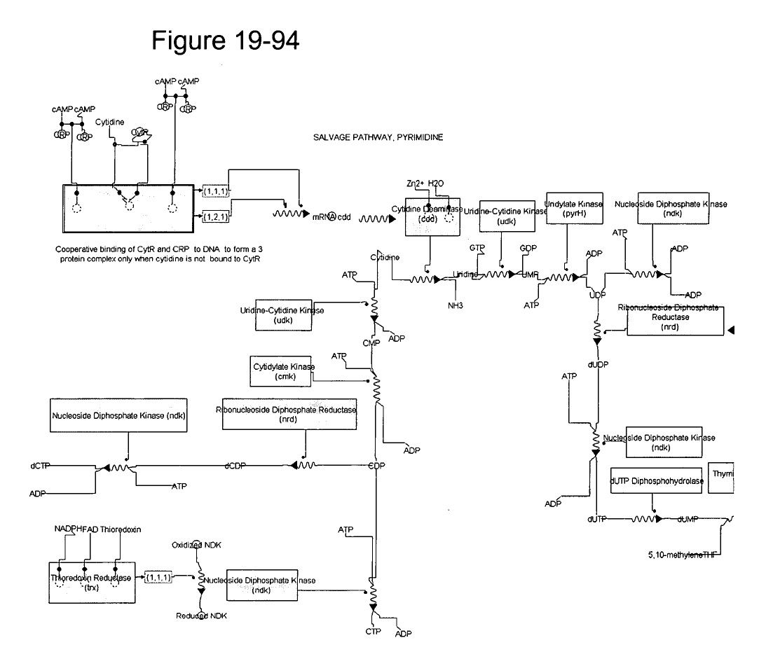

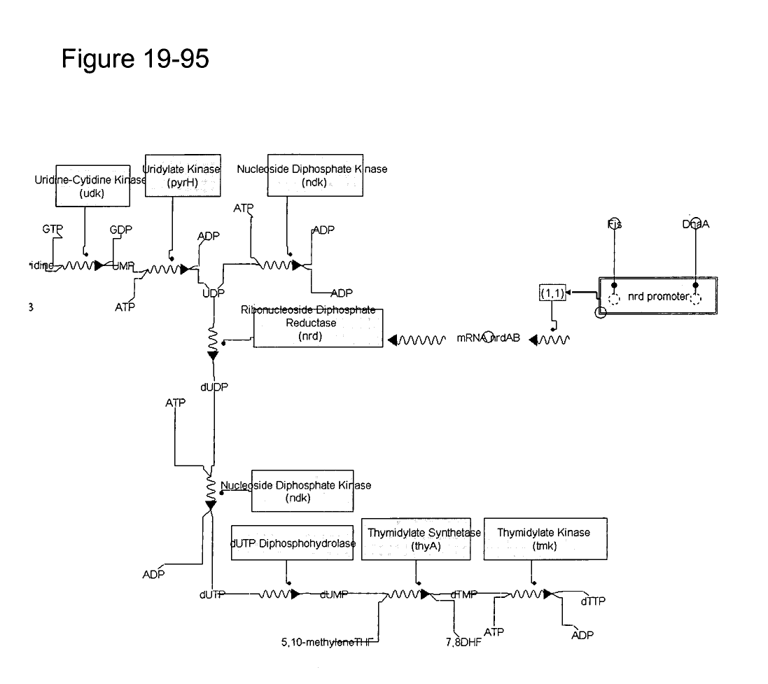

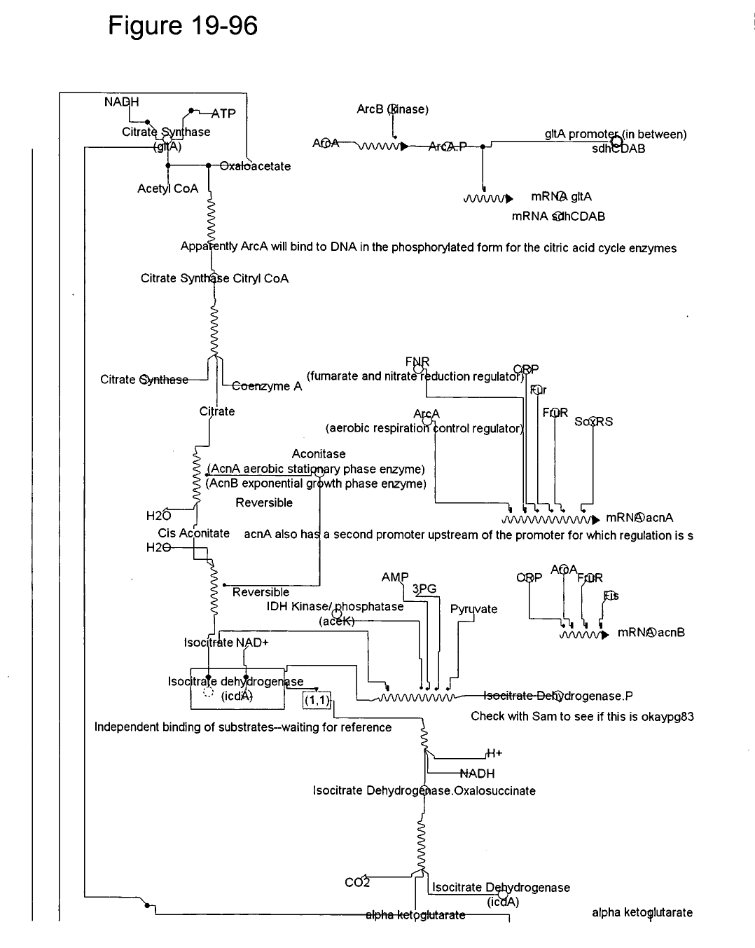

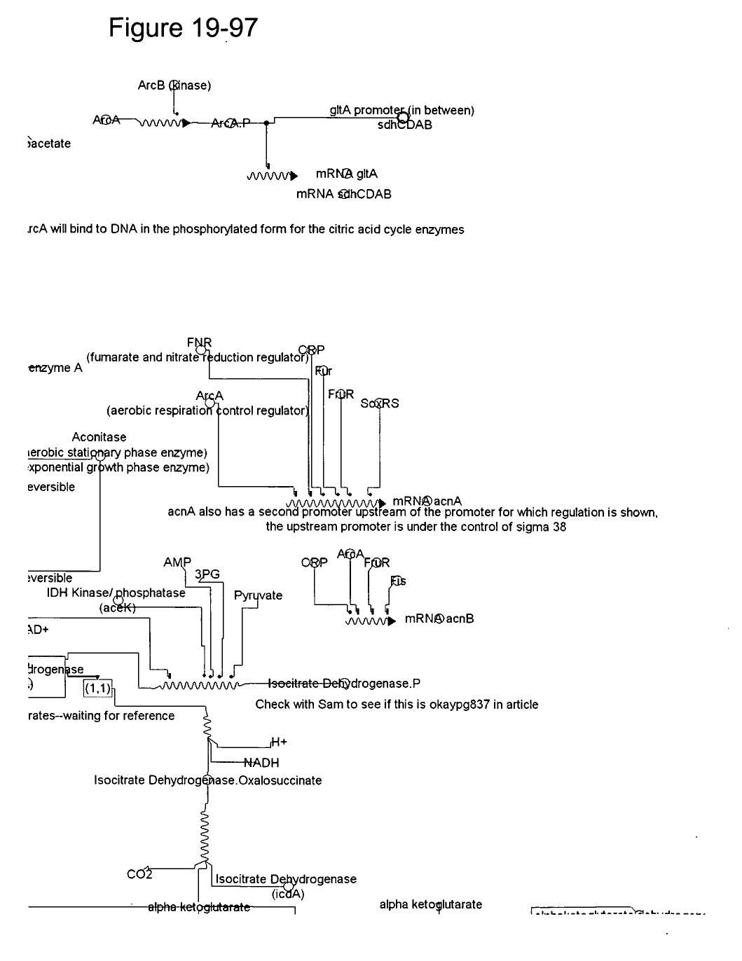

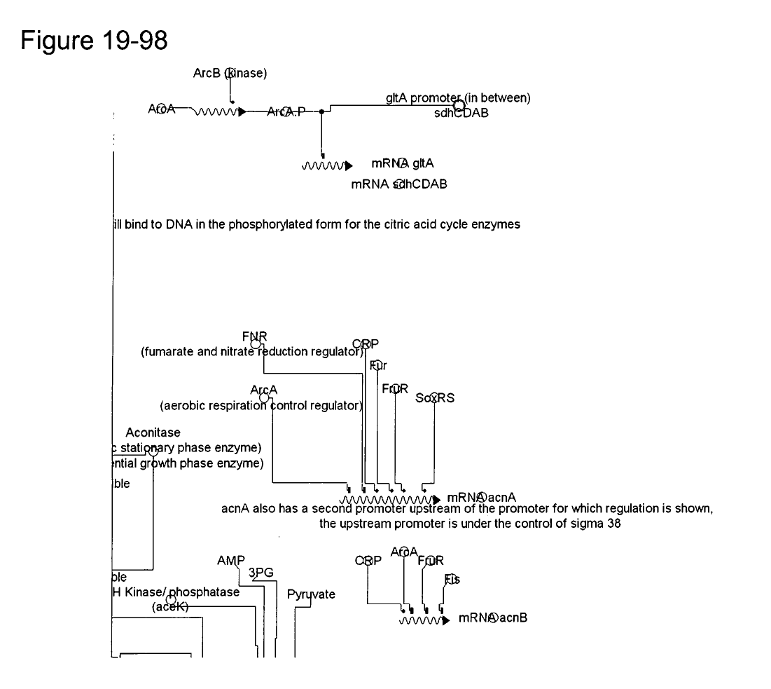

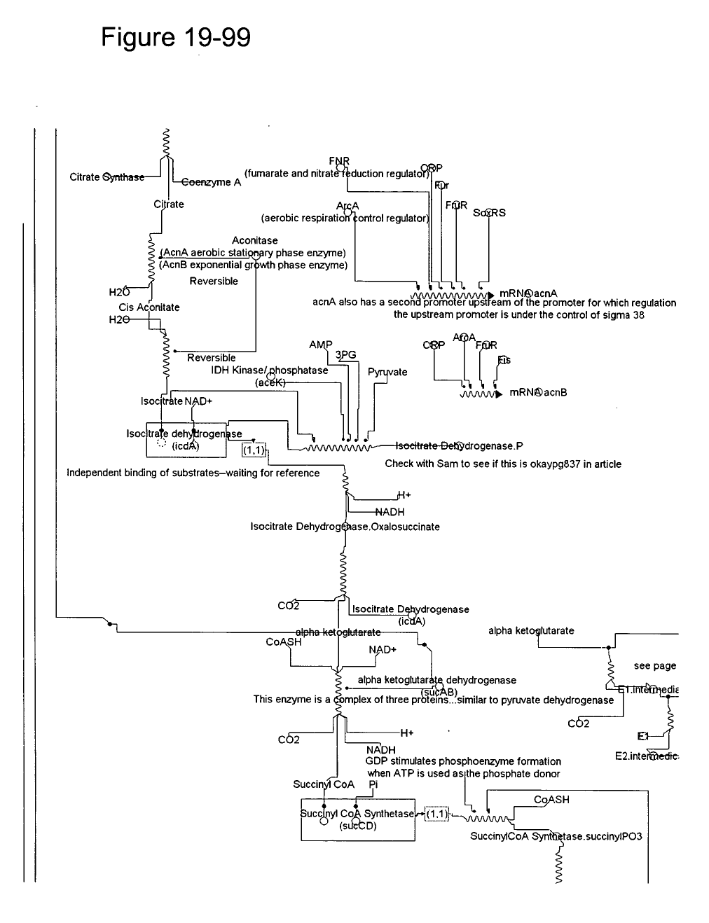

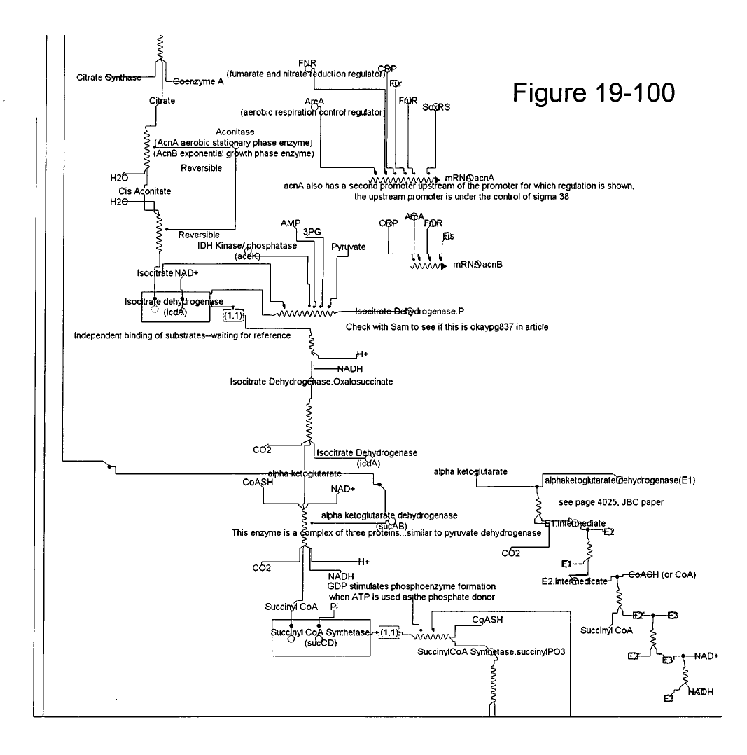

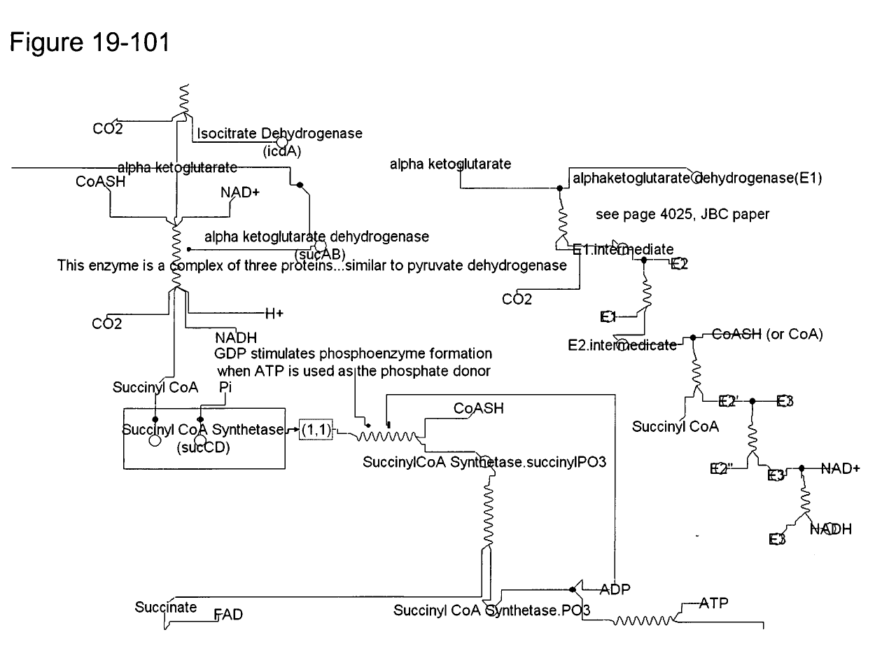

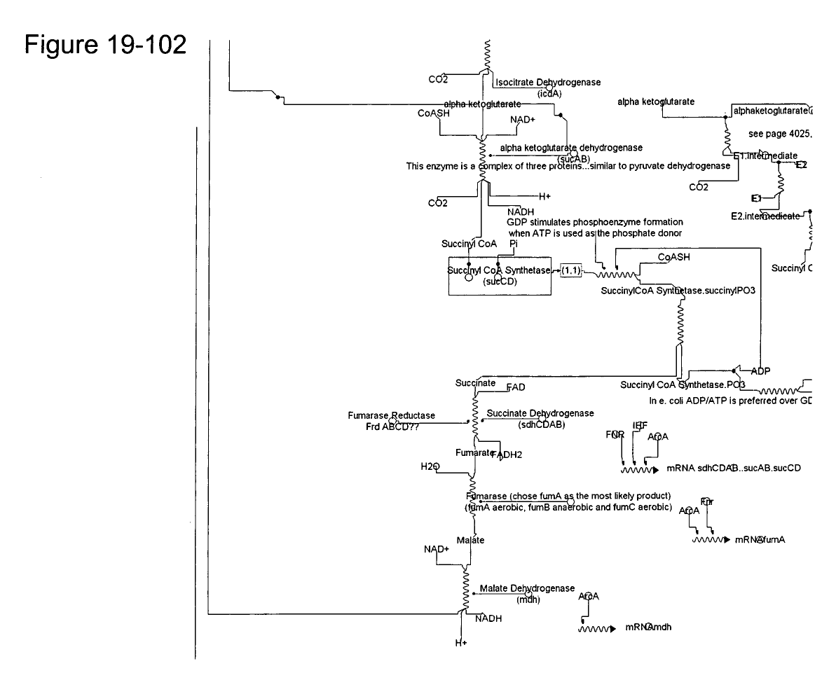

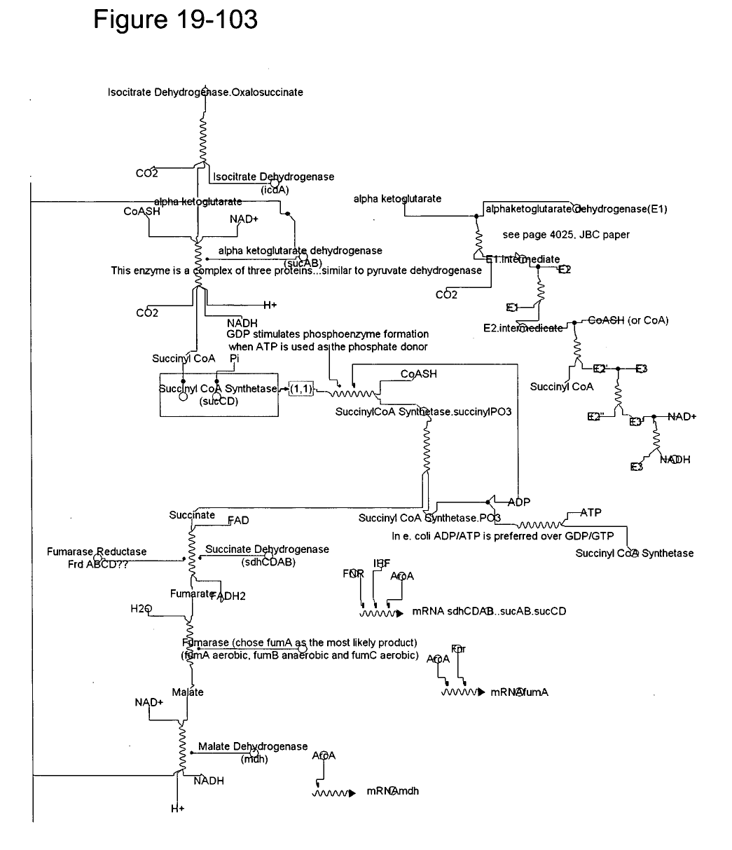

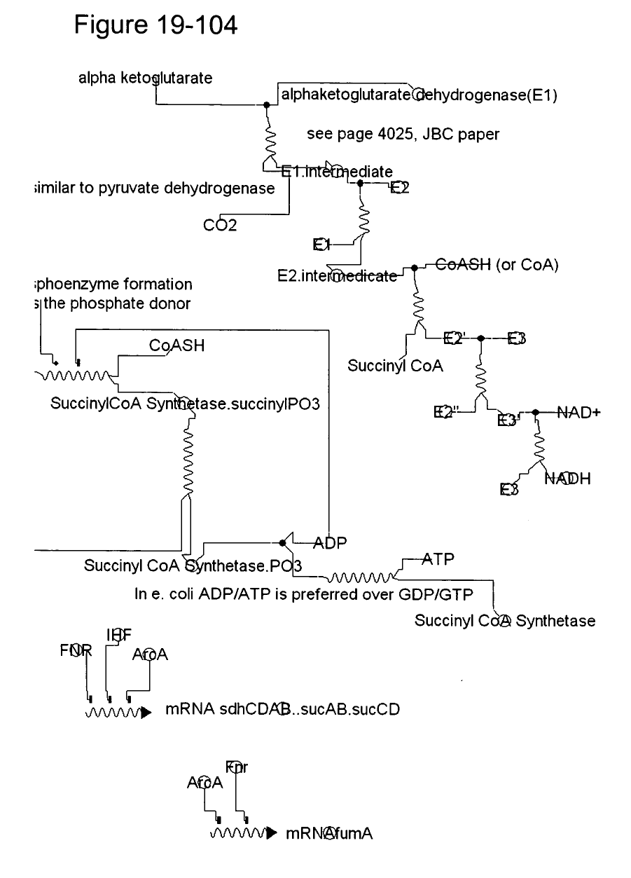

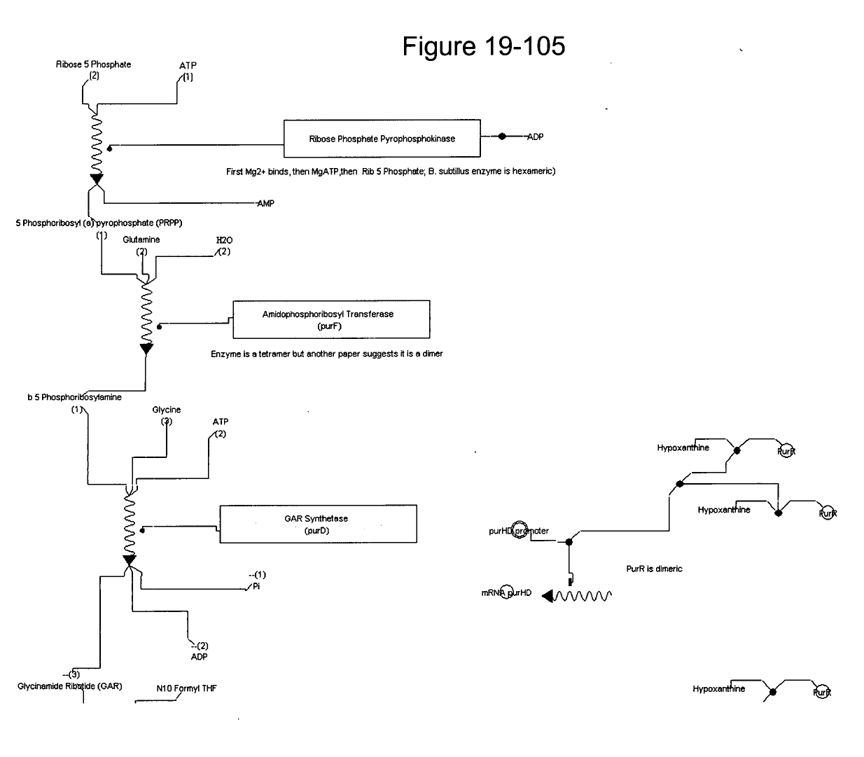

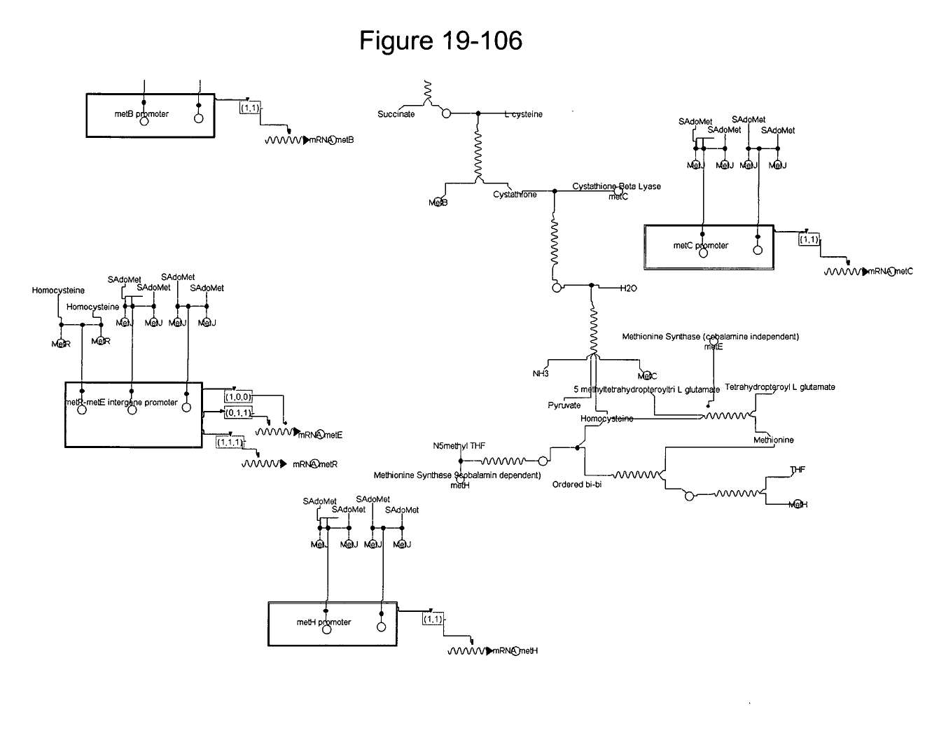

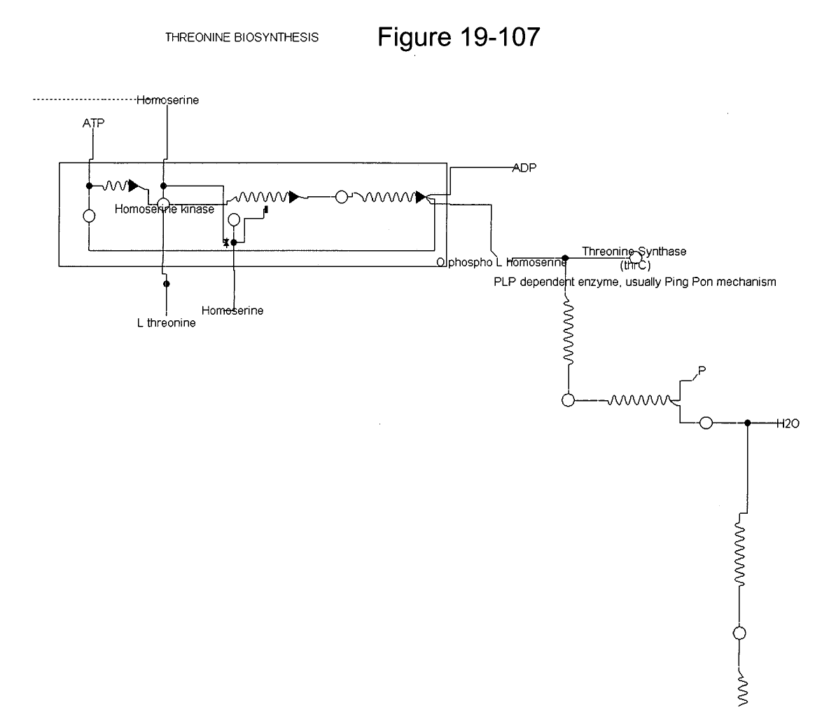

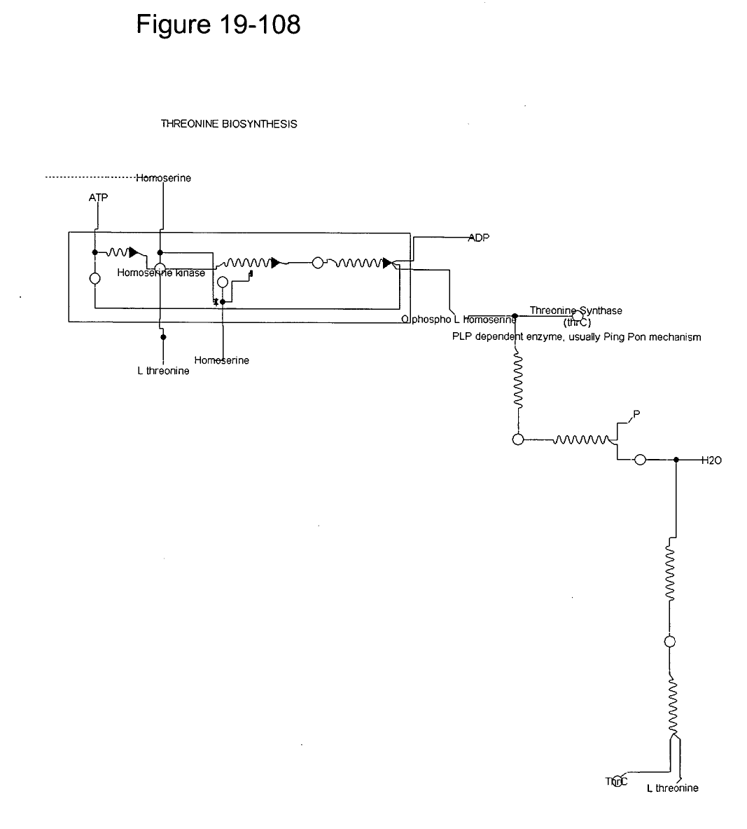

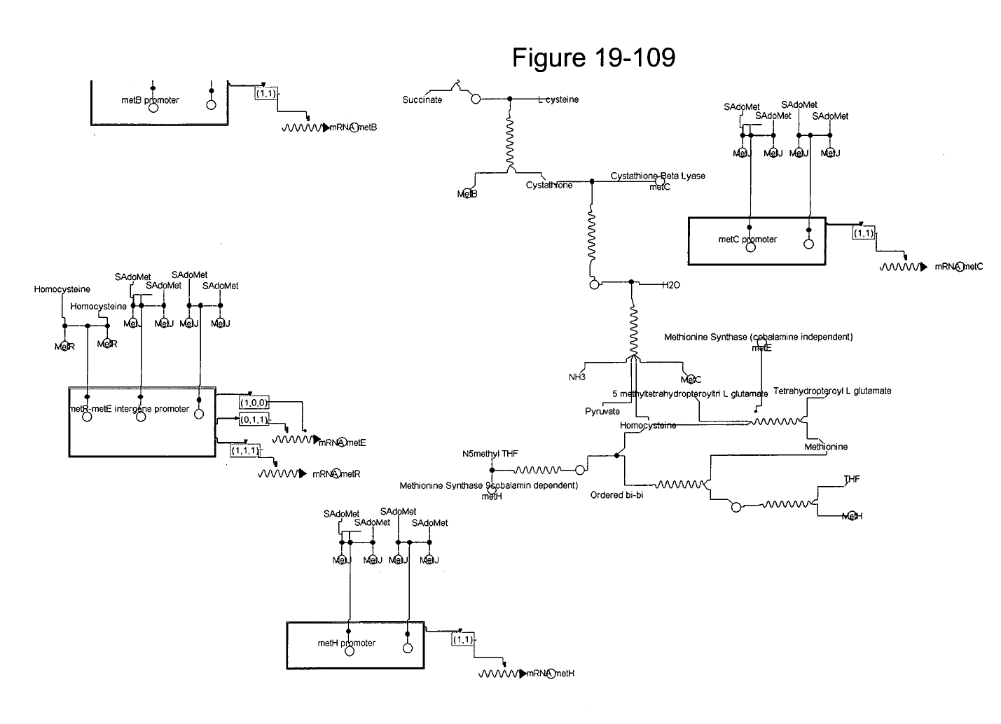

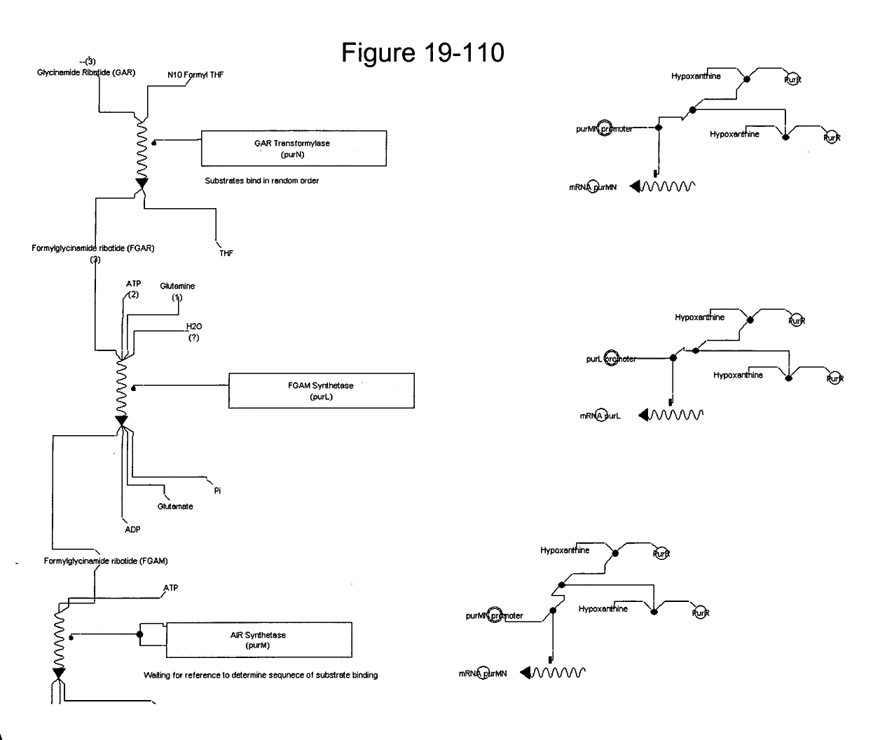

From the DLC representation of the model, one can then translate the representation to a set of reaction steps and their corresponding differential equation solution, stochastic analogues, or a hybrid solution. Of course, can go directly to reaction steps and by pass the DCL representation. This approach can easily be done for model with a small number of components and interactions, but is not efficient for getting at the level of hundreds to thousands of components and their interactions. FIGS. 19-1 through 19-110 show the DCL representation for a subset of the pathways in Table 1.1.

Specific Model of the Lysine Pathway:

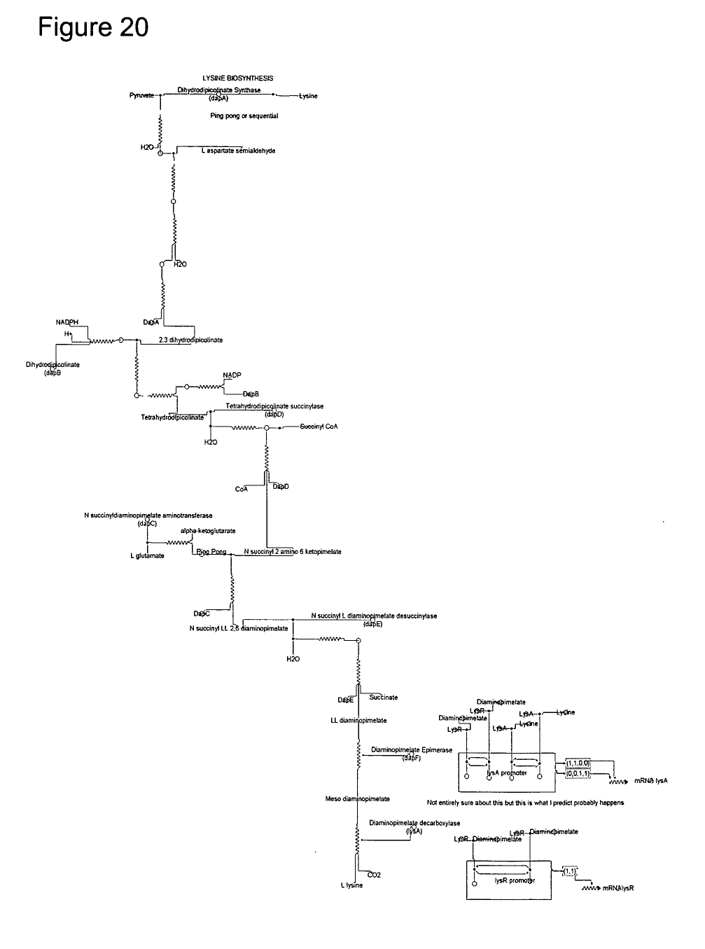

To illustrate the modeling of the pathways in the E. coli model, consider the lysine pathway shown in FIG. 20. This pathway is part of a larger pathway that includes lysine, threonine and methionine as its three end products shown in simplified form in FIG. 21.

The first two steps in the pathway that convert L aspartate to L aspartate semialdehyde are shared. The lysine pathway branches off after the second step while methionine and threonine pathways proceed for another shared step before separating into unique biosynthetic steps.

The pathway is regulated at three steps:

1. The first reaction, aspatokinase or aspartate kinase, is catalyzed by three isozymes, AKI, AKII and AKIII, each of which is feedback inhibited by either lysine, threonine or methionine. The lysine-sensitive isozyme in this case is LysC.

2. The first unique enzyme for lysine, Dihydropicolinate Synthase (DapA), is negatively inhibited by high concentrations of lysine.

3. The last reaction in the pathway, Diaminopimelate Epimerase (LysA), catalyzes decarboxylation of the intermediate meso-diaminopimelate to form L-lysine. This enzyme is regulated at the level of transcription by the transcriptional activator LysR and is autoregulated. In addition, there is evidence that the synthesis of the enzyme is repressed by the end product lysine and stimulated by diaminopimelate.

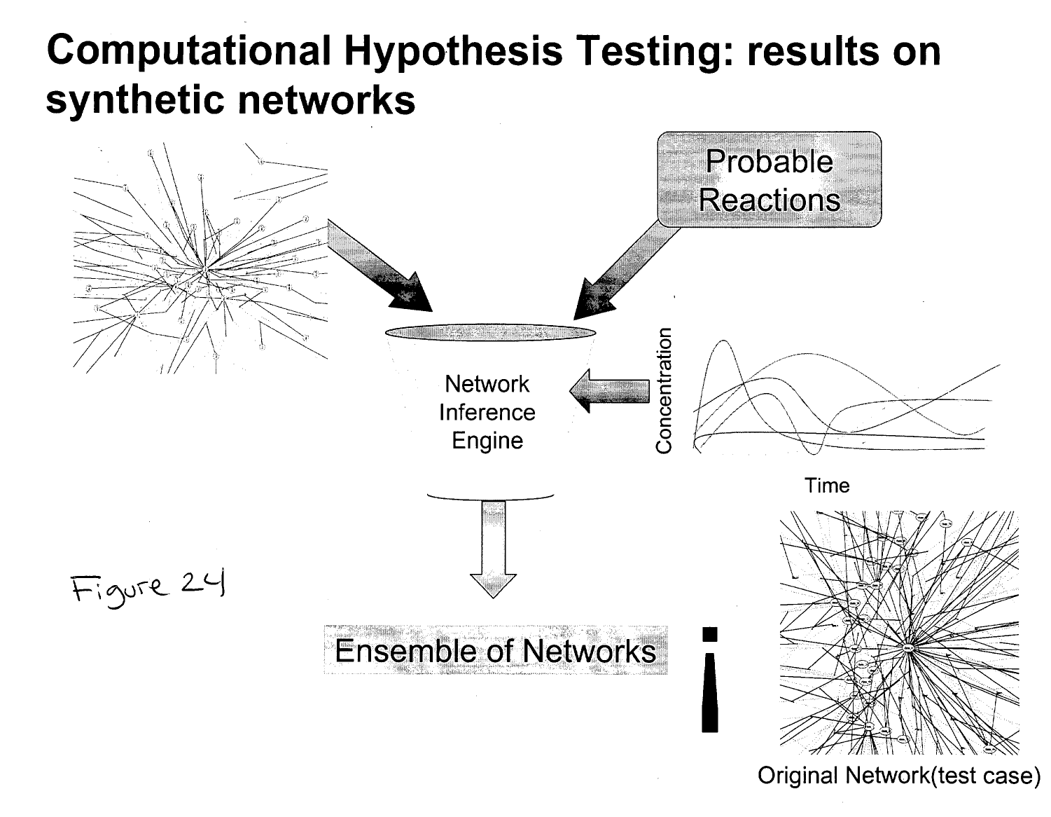

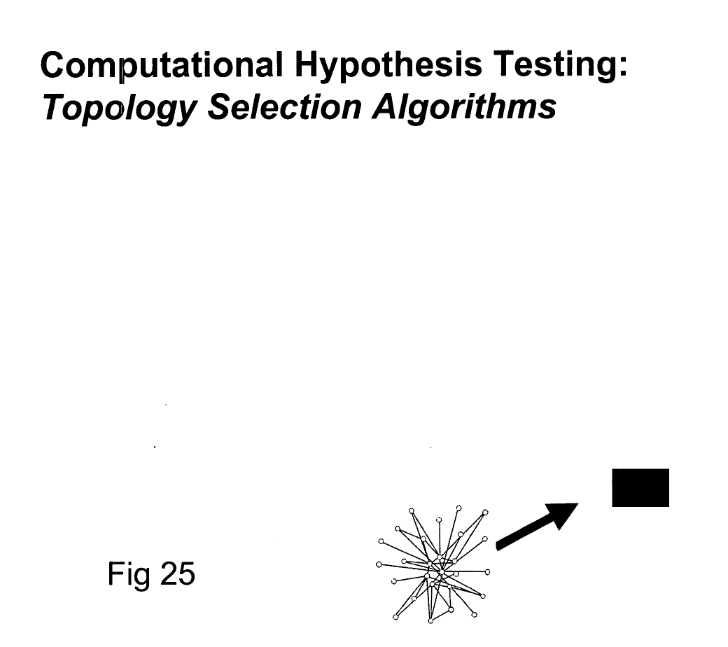

From the DCL representation in FIG. 25 one can then easily write down the reaction steps of the pathways and then assign corresponding kinetic forms as described in the detailed methodology. This can also be done automatically via then VisualCell/DigitalCell interface.

The rate constants are given initial values based on values founding the literature. For rate constants that are not known values are chosen that are typical of other components in E. coli. The total number of parameters is 64 in the lysine pathway. The conversion of the reactions steps into differential equations was automatically done by the Digital Cell and is shown in exhibit A along with parameter values chosen. These equations represent concentrations of individual components or intermediates, processes such as transcription, translation, degradation, as well as all the aspects of regulation mentioned previously. The simulation output of the pathway is shown in FIG. 22.

This procedure is repeated for all of the pathways listed in Table 1.1 where components that are common to the pathways represented major nodes of cross talk between the pathways. As a strategy each pathway is modeled separately and then the pathway modules are connected via their common nodes to create comprehensive whole cell E. coli model.

Adding Physiological Processes to the Model

It is not enough to create a comprehensive and connected model of the pathways controlling the physiological processes. These pathways have to be connected to the physiological processes of cell growth, cell division, DNA replication, cell volume change, and changes in cell shape. In this way the model can predict molecular response and physiological response. The E. coli modular cell model of Shuler et. al. provides a guide for connecting the detailed molecular response to cell physiology.

Specific pathways play important roles in cellular processes. For instance, during DNA replication, nucleotides generated by the purine and pyrimidine biosynthesis pathways are used for synthesizing the new DNA strand. Similarly, the end products of those pathways are also used during transcription when new mRNA is transcribed in response to specific regulatory signal.

In the presence of the available inputs, glucose and nitrogen, the single cell synthesizes precursors and uses the precursors to synthesize macromolecules. At certain time intervals, the sum of all of the molecules in the simulation is computed to get at the number of molecules after cell growth is initiated via the addition of Glucose and Nitrogen. From this one can derive the mass of the cell based on the molecular weight of the componets and derive the volume from the average density of the E. coli where:

Volume=total mass/density.

Increased mass or biomass causes the cell to grow in size and increase in volume. In the simulation, once the cell reaches a certain size (roughly 1.5 the original, at twice the volume that is its cue to divide in two), the process of DNA replication begins and then starts septum formation in preparation for cell division.

The E. coli model can predict cell geometry. The cell can be thought of as a cylinder and when the septum forms, it divides the cylinder into 2 hemispheres. Cell cylinder length and width are calculated using cell surface area, septum surface area and cell volume. Cell surface area is calculated from the amount of cell envelope material and the cell envelope area density. Cell envelop material is one of the cellular constituents that is produced when the E. coli is given the inpute of Glucos and Nitrogen. Cell volume is computed from the cell mass, cytoplamic density and envelope density.

Training the E. coli Model to Optimize Kinetic Parameters and Account for Unknown Genes and Proteins

Here, one can collect quantitative dynamical time course measurements, static measurements, and phenotypic measurements on the response of E. coli to various perturbations (Glucose addition, different levels, other stimuli listed in Table 1.2, knockouts, tranfections, . . . etc.). The data can be collected on RNA, protein, and metabolite measurements. The optimization algorithms can train over multiple data sets and conditions.

Bacteria is a good system for conducting bioinformatics anlaysis to generate hypothetical interactions since one has access to multiple genomes. For example, upstream regions in DNA sequence over multiple organisms can be analyzed for transcription binding motifs. The sequence analysis can also be combined with analysis of high-throughput measurements to generate additional links. Predictions can be normalized using above mentioned methods. From this one then construct a Probable Reactions database on hypothetical interactions in E. coli.

The core E. coli whole cell model, the Probable Reactions database for E. coli, can then be inputted into the Computational Hypothesis Testing framework to generate ensemble models that best match the data, that can predict functionality of unknown genes and their interactions, and predict phenotypic response under various conditions. Of the over 4,000 genes in E. coli, roughly 30-40% have unknown function.

Whole Cell Simulation of a Mammalian Cell

A description of building a complete large-scale data driven simulation of a cancer cell is provided. The methodology described here is general and can be applied to modeling any mammalian cell.

Creation of the Core Cancer Cell Simulation:

The goal is to create a core simulation that would contain the most relevant genes and proteins for describing the mammalian cell cycle and cancer progression. While this is a simulation of a cancer cell, it can also be applied to normal cells. One simply takes the core cancer simulation and trains it on normal data to get a simulation that applies to normal cell. This cell model would contain a minimum of the following pathways:

1. Signal transduction pathways controlling cellular proliferation and survival: such as RasMapK, Wnt-beta catenin, P13K, NfkappaB etc.

2. Cell cycle progression: G1-S transition, G2-M transition, S phase; check points such as DNA replication and repair, chromosomal segregation, highly connected p53 node

3. Cytokine pathways controlling stress response and growth: IL's, JNK, p38, Stats

4. Cell death pathways: apoptosis, necrosis

5. Angiogensis (process by which tumors develop a blood supply)

6. Metastasis (process by which cancer cells detach from the tumor and invade other parts of the body)

7. Immune surveillance

8. Metabolic pathways

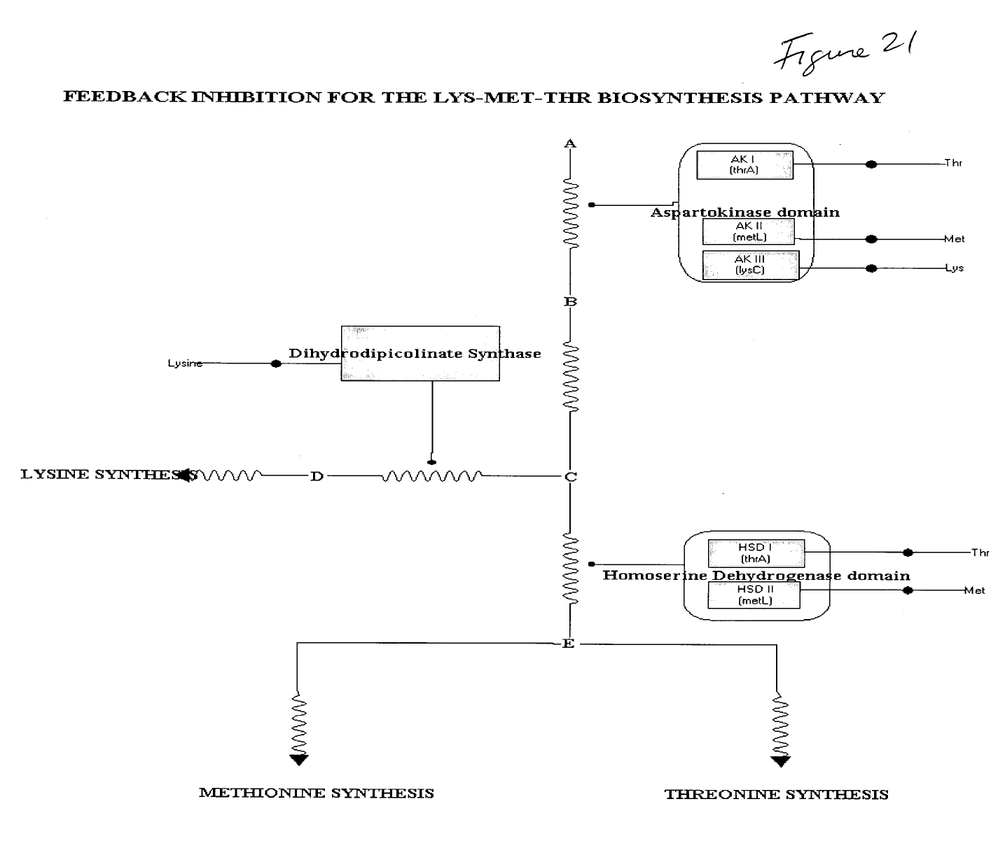

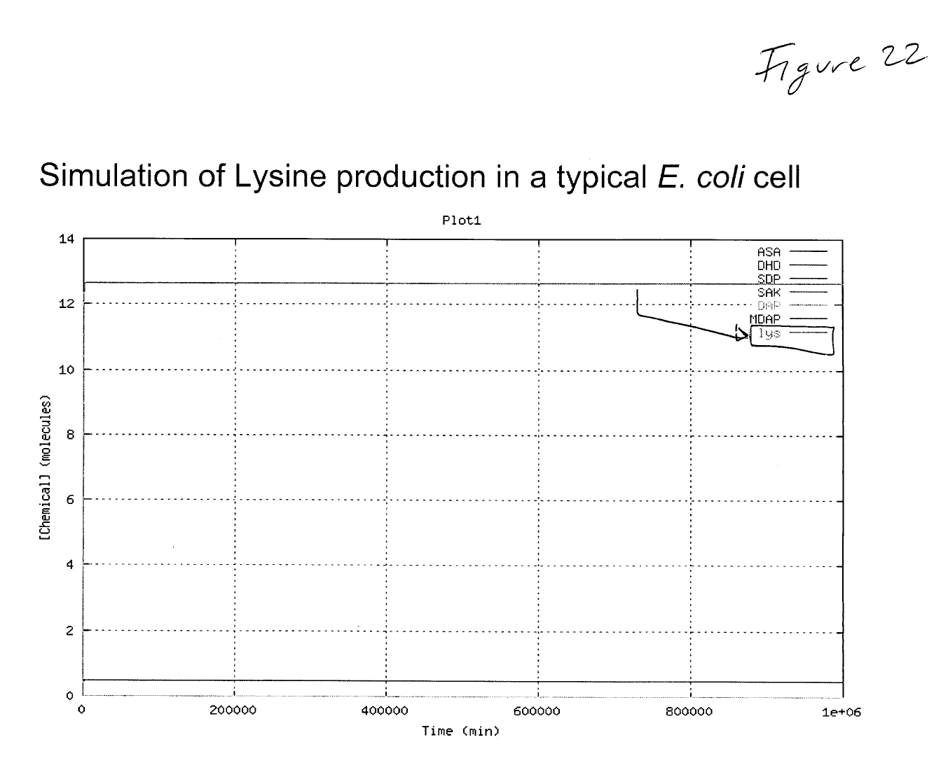

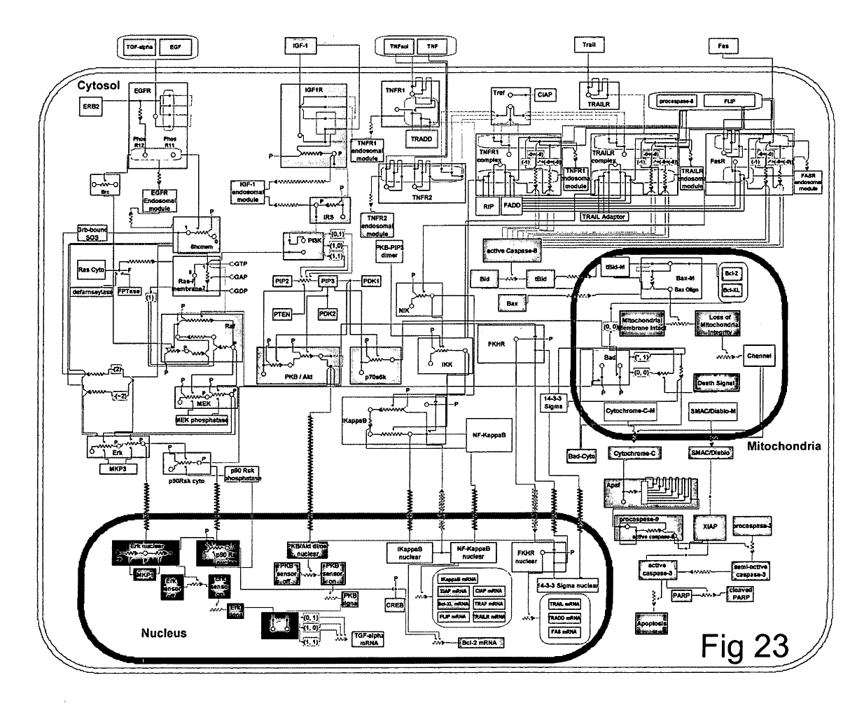

Biologists mine the literature and rate the interactions (similar to what was described above for E. coli) keeping highly rated links. Poor links or links with poor experimental evidence in the literature is deposited in the probable reactions database to be tested in an inferential manner. The model is created in DCL to allow for concise and scaleable representation. FIG. 23 contains a portion of the DCL representation of signal trandsduction pathways underling cell growth and proliferation and apoptosis. A translation of the full DCL model is in Exhibit B and contains over 500 genes and proteins and at least five times the number of states. A state is defined as the various modifications a component can exist in such as a protein that is phosphoryalted, bound, etc. Using DCL the model can be scaled to include other pathways listed in 1-8.

The model contains the various reactions that gene and proteins can undergo, including the of processes receptor activation, degradation, endocytosis, transcriptional control, transport within compartments, protein translation and degradation. For transport, compartments are added to simulate spatial aspects of the cell. For example, this cell model has six compartments (other can be added to account for spatial localization and translocation). Components can be in the cell membrane, endosome, mitochondria membrane, mitochondria, nucleus, and the cytosol. Some components in the cell model are localized while others can translocate between the various compartments. Physical location and translocation is simply modeled as another state. A component A can be in the cytosol, when it translocated to the nucleus it becomes A_nuclear and can be modeled with the kinetic form:

[A] kt_-A_nuclear

and stoichiometry

1 molecule of [A] removed, 1 molecule of [A_nuclear] created.

Another way to model translocations is via a time delay equation. Because translocations involve many reaction steps where the reaction steps are not known in detail, a time delay maybe required. In which case the kinetic form is:

[A(t-delta)] kt_A_nuclear,

where delta is the time delay parameter. In general, whenever a reaction involves many intermediate reaction steps that are not known or it is not convenient to model them, a time delay can realistically substitute for those reactions. In addition continious events are coupled to discrete stochastic events to model physical processes such as mitochondrial disruption by a protein called Bax. When certain apoptotic pathways, Bax is activated via translocation to the outer mitochondria and oligomerization. Bax in the oligomerized form acts to somehow break the mitochondria integrity. Thus in the model there is a component called mitochondrial integrity that gets degraded away by Bax, when the degradation falls below a certain threshold, then the mitochondria is disrupted and cytocrome c is released. The discrete event is modeled as a “Switch” function.

One can then translate the DCL representation into reaction steps that a simulation engine can solve such as the DigitalCell. Exhibit B contains the differential equations underlying the DCL FIG. 23.

Network Inference Results on Synthetic Networks

As proof of concept, the full Computational Hypothesis Testing program is tested on synthetic networks. The term synthetic here is used to refer to networks that have no biological basis, but are simply a network with a given number of components and interactions between the components reflecting some kind of biological network.

A synthetic network of 25 nodes with various interactions between the nodes is constructed. For example, node 1 and node 2 may bind and produce an output that is node 1 again and node 3 (similar to an enzyme catalyzed reaction in a biological system). The network is simulated and data is generated to represent the kind of data output one would get from a real biological network. To further represent the kind of system one encounters, nodes and interactions are deleted so that roughly half of the network is missing. One then generates a probable reactions database with false and true reactions (similar to what one would generate by mining sequence and experimental data in a biological system).

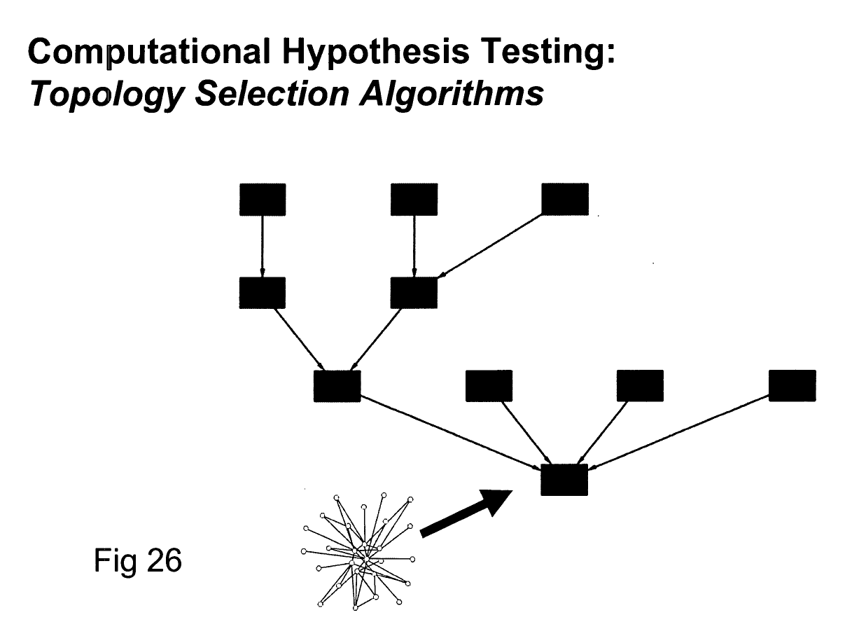

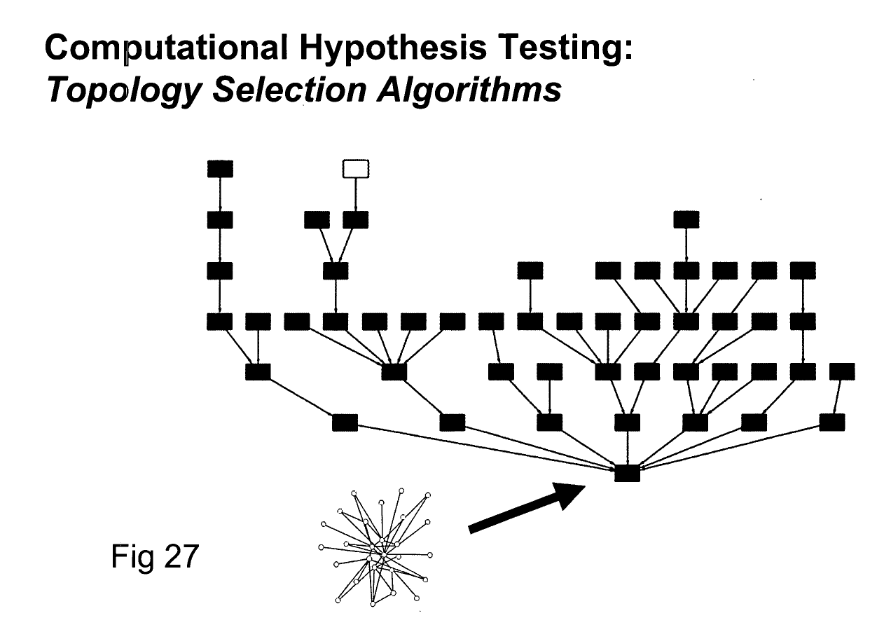

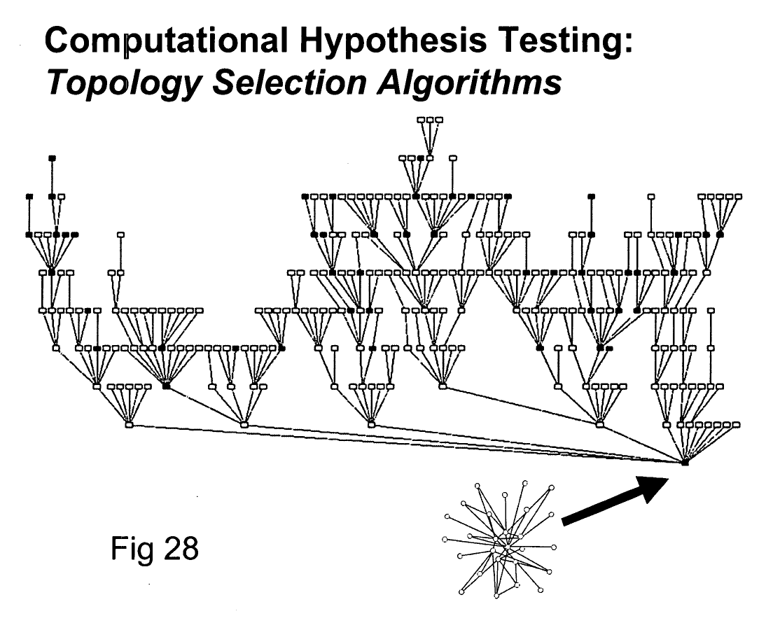

A Computational Hypothesis Testing algorithm is written that takes as input the synthetic network with roughly half of the nodes and interactions missing (hence forth referred to as a sparse network), a set of Probable Reactions, and the generated experimental time course measurements (as shown in FIG. 24). The algorithms begin with the sparse network and iteratively loop through the interactions in the Probable Cell database. Networks that improve the cost are kept and are continually evolved with components and interactions added and removed. A depiction of the process is shown in FIGS. 25-28, where the blue node represents the starting sparse network. Subsequent images show the blue node evolving where only the best performing networks are kept. FIGS. 29-30 contains the output of the algorithm average over the best performing networks. The curve labeled “Experimental data profile” refers to the generated data from the original network and the curve labeled “Inferred profile” refers to the output of the Computational Hypothesis Testing algorithms averaged over the best performing networks in a cost weighted manner. As is apparent from the figures, the algorithm is able to predict the trends in the data and is even able to predict the simulation output for a node where there was no observed data.

1.7 Applications of the Model

A predictive model of the biological system provides a dynamical, functional framework for housing the genetic and molecular knowledge underlying the system. Dynamical models are transparent in their inner workings and allow researchers to explicitly perturb the model to reflect a specific type of biological state and to perturb the system to identify which components govern the physiological behavior of the system.

Once the simulation is created and optimized using the techniques of (a)-(e) in section 1.0 (where each item is described in further detail in subsequent section) it can then be used in a variety of ways. The level of optimization can be up to any one of the techniques in (a)-(e), but of course the further one along is in the process the more predictive the simulation will be (henceforth, an optimized simulation refers to a model built using the techniques (a)-(e) to any level). In general the simulation has a number of uses applicable to a number of areas from drug discovery to industrial production.

The general applications of the model fall under the heading (I) prediction of phenotypic outcome; (II) elucidating which components to measure in order to determine phenotypic outcome; (III) discovering new biological pathways in the system; (IV) elucidating the function of unknown or poorly characterized genes, proteins, or other cellular components; the (V) elucidating the mechanism of action of entities used to perturb the system (such as a drug); and the (VI) tailoring (I)-(V) to match a particular genetic pathology of the cell or biological system. Phenotypic outcome is defined as the actual response of the cell or biological system to given perturbation. It can refer to the actual physiological state that the system is in, such as whether or not a cell is in any one of its cell cycle phases (G1-S, G2-M, S phase, . . . etc.) and or the expression profiles of constituents in the system such as RNA levels and protein levels.

Exemplary methods of the invention for (I) prediction of phenotypic outcome, starting from the optimized simulation of the cell or biological system comprising the techniques of:

A. specifying a stable phenotype of the cell or biological system simulation;

B. correlating that phenotype to the state of the cell or biological system simulation and the role of that cellular state to its operation;

C. specifying a cellular biochemical networks and chemical states outputted by the simulation believed to be intrinsic to that phenotype;

D. perturbing the characterized network by deleting one or more components thereof or changing the concentration of one or more components thereof or modifying one or more mathematical equations representing interrelationships between one or more of the components; and

E. solving the equations representing the perturbed network to determine whether the perturbation is likely to cause the transition of the cell or biological system from one phenotype to another, and thereby identifying one or more components for interaction with one or more agents.

An obvious extension of the above methodology is to use the simulation to determine the reverse, which components to perturb in order to lead to the desired phenotypic outcome. In addition, you can test out various concentrations and kinetics of interaction to not only determine which perturbation, but the concentration and parameter values you need to choose to get at the desired phenotypic outcome.

Exemplary methods of the invention for (II) elucidating which components to measure in order to determine phenotypic outcome starting from the optimized simulation of the cell or biological system, comprising the techniques of:

A. perturbing the system to generate the desired phenotypic state using the above method for (1) prediction of phenotypic outcome, starting from the optimized simulation of the cell or biological system

B. determining a control phenotype upon which to compare the desired phenotype with

C. solving the equations representing the perturbed network and the control network

D. identifying chemical species that show a difference in expression between the perturbed and control cell or biological system

E. using these chemical species as markers for distinguishing between the perturbed state and the control state in an experimental assay

F. using the measurement to optimize or re-optimize the simulation then predict phenotypic outcome as described in (I)

Exemplary methods of the invention for (III) discovering new biological pathways in the system, comprise the techniques of:

A. creating an optimized simulation of cell or biological system using the methodology described above

B. generating possible set of interactions between possible components in the undiscovered pathway and inputting it into the probable reactions database

a. using data-mining and bioinformatics tools

b. experimental assays that hint at possible interactions such as high-throughput measurement methods that generate many possibilities directly or via further bioinformatics analysis, some false some true (e.g. yeast two-hybrid, DNA microarrrays, protein arrays, . . . etc.)

c. a combination of (a) and (b)

C. collecting quantitative and qualitative experimental data on possible constituents in the unknown pathway or other constituents that interact with the unknown pathway

D. integrating the core simulation, the probable reactions database, and static and dynamical time course measurements into the Computational Hypothesis testing framework

E. generating an ensemble of biological network structures of the previously unknown pathway

F. probabilistically rating the predicted components and their interactions in the pathway based on:

a. the generated population of networks, giving higher weight to components and interactions that appear in a majority of the population

b. the cost criteria assigned to predicted interactions in the pathway from the Computational Hypothesis testing framework

G. testing the predicted interactions and pathways by perturbing the simulation and comparing to experimental data

H. and or validating predicted interactions of the entity in the cell with the highest probability via direct experimental measurement of the interaction

Exemplary methods of the invention for (IV) elucidating the function of unknown or poorly characterized genes, proteins, or other cellular components comprise the techniques of:

A. creating an optimized simulation of cell or biological system using the methodology described above

B. generating possible set of interactions between unknown component and other components in the simulation and components not yet in the simulation and inputting it into the probable reactions database

a. using data-mining and bioinformatics tools

b. experimental assays that hint at possible interactions such as high-throughput measurement methods that generate many possibilities directly or via further bioinformatics analhysis, some false some true (e.g. yeast two-hybrid, DNA microarrrays, protein arrays, . . . etc.)

c. a combination of (a) and (b)

C. collecting quantitative and qualitative experimental data on unknown components and other constituents that interact with the unknown components

D. integrating the core simulation, the probable reactions database, and static and dynamical time course measurements into the Computational Hypothesis testing framework

E. generating an ensemble of biological network structures with the previously unknown components

F. probabilistically rating the predicted components and their interactions in the pathway based on:

a. the generated population of networks, giving higher weight to components and interactions that appear in a majority of the population

b. the cost criteria assigned to predicted interactions in the pathway from the Computational Hypothesis testing framework

G. testing the predicted interactions and pathways by perturbing the simulation and comparing to experimental data

H. and or validating predicted interactions of the entity in the cell with the highest probability via direct experimental measurement of the interaction Exemplary methods of the invention for (V) elucidating the mechanism of action of entities used to perturb the system (such as a drug), comprise the techniques of:

A. creating an optimized simulation of cell or biological system using the methodology described above

B. generating possible set of interactions between the entity used to perturb the system in the undiscovered pathway and inputting it into the probable reactions database

C. using data-mining and bioinformatics tools

D. experimental assays that hint at possible interactions such as high-throughput measurement methods that generate many possibilities directly or via further bioinformatics analysis, some false some true (e.g. yeast two-hybrid, DNA microarrrays, protein arrays, . . . etc.)

E. a combination of (a) and (b)

F. collecting quantitative and qualitative experimental data on components in the cell under the effect of the entity used to perturb the system (experimental measurements that determine the quantitative and qualitative response of the system to the unknown entity)

G. integrating the core simulation, the probable reactions database, and static and dynamical time course measurements into the Computational Hypothesis testing framework

H. generating an ensemble of biological network structures of the previously unknown pathway

I. probabilistically rating the predicted interactions of the entity with components in the cell based on:

a. the generated population of networks, giving higher weight to components and interactions that appear in a majority of the population

b. the cost criteria assigned to predicted interactions in the pathway from the Computational Hypothesis testing framework

J. testing the predicted interactions of the entity by perturbing the simulation and comparing to experimental data

K. and or validating predicted interactions of the entity in the cell with the highest probability via direct experimental measurement of the interaction

Exemplary methods of the invention for (VI) tailoring I-V to match a particular genetic pathology of the cell or biological system, comprise the techniques of:

A. determine genetic pathology of cell or biological system by:

a. identifying which components (genes, proteins, . . . etc.) are mutated and as a result have different expression and kinetics than the un-mutated components. This can be done via direct sequencing of genes, direct measurements of the kinetics and dynamics of genes and proteins

b. optimize or re-optimize cell or biological system simulation to data on specific genetic pathology

B. use this model to make specific predictions on the genetic pathology of interest as described in 1-5.

The above methodologies can then be applied to the areas of drug discovery, clinical diagnosis and treatment, genetic analysis, bioremediation, optimization of bioreactors and fermentation processes, biofilm formation, antimicrobial agents, biosensors, biodefense, and other applications involving perturbing biological systems.

Drug Discovery

A Mammalian cell simulation of different cells in the human body can be applied to a number of diseases effecting the regulation of the Mammalian cell cycle such as cancer, Alzheimer's, arthritis, neurodegenerative diseases, amongst others. The whole cell simulation can also serve as a template for building models of populations of cells, populations of cells representing organs and tissue, populations of cells representing the diseased cell mass (e.g. a tumor), and the combination of the two to model the organ or tissue interaction in the presence of the diseased cell mass and other cells in the body that interact with them (e.g. immune cells). This means that the models created via this methodology include predictions on the single cell level (most relevant to cell lines), populations of cells (most relevant to cell lines and cell populations in the body), to the organ level (relevant to cell lines, animals, and humans), and eventually the whole human level where a broad spectrum of cell types, organs, and the interactions thereof is being modeled.

The drug discovery process involves many phases starting from target and lead compound discovery, to lead compound optimization, to efficacy and toxicity studies in animals, to clinical trials in humans. It is expected though, through optimization of the simulation via inclusion of more and more data and iterative refinement of the simulation that the models will get better and more predictive on the clinical in vivo level. When this happens, many of the linear techniques of drug discovery occur in parallel shortening the length of time it takes to get a drug to market (currently an average of over 15 years). Below is a description of how the simulation can be used to enhance each stage of drug discovery.

Target and lead compound discovery: researchers need to determine the target or targets that needs to be perturbed in order to reverse the disease state. Tests are mainly conducted on cell lines where the single cell models and population of cell models can be used to derive predictions.

Lead compound optimization: once a lead compound has been selected the lead compound can be tested within the simulation and used to determine phenotypic response and mechanism in the general methodology section for the application. One important use is to determine if the un-intended targets of the drug actually improve the efficacy of the drug or not. The drug can then be optimized to either avoid hitting the un-intended target or left alone as assessed by model prediction that it would lead to enhanced efficacy if it did hit the un-intended target. Within the simulation one can also perturb the kinetics of the lead compound with the target or targets it is hitting to determine the optimal kinetics it should have. This similarly applies to other treatment methods such as antisense therapies.

Animal studies: here researchers want to assess the efficacy and toxicity of the lead compound or a given therapy (e.g. antisense, antiobody, . . . ) in an animal such as the mouse as an indicator of what would happen in human. Eventually, once the simulations get accurate enough via optimization and iterative refinement to enough human data, they may actually replace animal studies. In the meantime the simulation can be used to improve the assessment resulting from animal studies. For example, a simulation of the animal cell model can be constructed and compared to the human cell model. One can use this to assess when the animal is expected to be a good indicator of human toxicity and when it is not. One can also use this to determine what are the important measurements to make in order to determine if the animal model is a good indicator of human efficacy and toxicity.

Phase I-III clinical trials: once a target or targets has been chosen and the corresponding therapy developed, then there remains the task of obtaining FDA approval of the therapy. Typically this involves efficacy and toxicity studies in populations of humans. The therapy has to be shown to be efficacious (a reversal or annihilation of the disease) while leaving the rest of the human healthy. Cell simulations of the diseased cell, tissue, or organ can be used as described to assess if the diseased state will be reversed. Cell simulations of various cell types in the human body such as the liver, kidney, heart, etc. and their extensions to the organ and whole human level can be used to assess toxicity.

An important aspect to clinical trials is to design the appropriate experiments for assessing efficacy and toxicity. There are three main aspects of experimental design involved:

a. Determining therapeutic dose: using the methodology on predicting phenotypic response to determine the optimal dose to use both for efficacy and reduced toxicity.

b. Therapeutic regimen—using the methodology on predicting phenotypic response to determine not just the dose, but a schedule as to how often and when the therapy should be given to lead to maximal efficacy and minimal toxicity.

c. Determining which populations will be responsive to the therapy and which ones will not based on various human genetic backgrounds.

d. Determining which molecular markers to measure (which components to measure) that would be indicative of efficacy and toxicity and the human population that the therapy is most effective against.

e. Finding a combination therapy (most likely of an already FDA approved drug) to combine your therapy with in clinical trials to show efficacy.

f. Using the simulation to rescues a failed drug by: determining optimal dose, therapeutic regimen, optimal population groups, a combination therapy

Phase IV: additional data is gathered in this stage and assessments are made for improvements or extending the uses of the drug, either singly or in combinations, to broaden the market. Using the simulation one can identify other diseases that it might be relevant to by testing it in other diseased cells. One can also determine other drugs it can be combined with to broaden the disease applications by testing those combinations in the simulation.

In more detail, outline the types of tests that can be conducted in the simulation. This can be in simulations on the single cell level, populations of cells, the organ level, or eventually the whole human. The type of model used depends on the stage of drug discovery as mentioned above. A drug, in this context, is used to refer to an agent that can perturb a target or targets in some way such as a small molecule inhibitor, antisense, . . . etc.

(1) Prediction of Phenotypic Outcome

Using the Methdology outlined above a research can predict the effect of phenotypic outcome on the disease state and compare it to the effect on the normal state. The testing can be done manually or in an automated fashion where an algorithm is used to perform the in silico tests in a high-throughput manner. The phenotypic state includes both the final physiological output of the cell (e.g. cell cycle arrest, apoptosis, or proliferation) or biological system and the genes and proteins that are affected as a result of the perturbation in the context of the entire circuitry of the cell. A researcher can then:

(i) predict the phenotypic outcome of perturbing a target or set of targets. The testing can be done manually or in an automatic fashion where an algorithm is programmed that tests the effects of perturbing the target in a particular disease state and also compares the outcome to the non-disease or normal state.

(ii) predict the phenotypic outcome of adding a drug or a set of drugs to the cell simulation. The testing can be done manually or in an automatic fashion where an algorithm is programmed that tests the effects of adding the lead compound or drug in a particular disease state and also compares the outcome to the non-disease or normal state.

(iii) predict the effect of perturbing a target or sets of targets in combination with another target or sets of targets

(iv) predict the effect of perturbing a target or sets of targets in combination with a drug or a set of drugs

(v) predict the effect of adding a drug or a set of drugs in combination with a drug or a set of drugs

(vi) predict the dose and timing of application of the drug and any combination that includes the above.

(2) Elucidating Which Components to Measure in Order to Determine Phenotypic Outcome;

The methodology can be used to design a measurement assay to determine the genes and proteins to measure in order to predict what would happen in a particular cell type, tissue type, or. a particular patient or groups of patients (other wise known as molecular markers).

(3) Discovering New Biological Pathways in the System;

The methodology can be used to find new pathways in the Mammalian cell system that could be of importance in a therapeutic area

(4) Elucidating the Function of Unknown or Poorly Characterized Genes and Protein or Other Cellular Components

The methodology can be used to uncover the functionality of unknown or poorly characterized genes and proteins that could be of importance in a therapeutic area. The sequencing of the entire human genome and DNA microarrays (which can lead to the discovery of groups of genes relevant for a disease process) has lead to the discovery of many genes and their protein products, but little knowledge of the functionality of those components.

(5) Elucidating the Mechanism of Action of Entities Used to Perturb the System (such as a Drug).

Once a target is found, a lead compound is identified that targets the particular gene or protein. Similarly, researchers often use a high-throughput compound screen to find lead compounds that induce the appropriate phenotypic response without necessarily having a particular target in mind (e.g. lead compounds that would lead to apoptosis in a cancer cell, but not a normal cell). In either case, the mechanism of action of the lead compound or drug may not be fully known. The methodology can be used to identify the cellular targets that the lead compound or drug is perturbing. This can also be applied to other treatment strategies such as RNAi, antiscence, and gene therapy. Again, some of these treatments would have the problem of non-specificity, but also of not knowing the effect that the single target perturbation has on the entire circuitry of the cell.

6) Tailoring 1-5 to Match a Particular Genetic Pathology of the Cell or Biological System

The above can all be tailored to reflect the genetic pathology of a particular cell type within a disease group in order to understand how applicable is the treatment or discovery to different genetic backgrounds. For example, a researcher may have ten different breast cancer cell lines each with a different type of mutation that gave rise to the breast cancer. Here they would want to use a simulation tailored to each cell line to derive the above-mentioned predictions. They may also be faced with testing the therapy against human populations with different mutations as identified by SNIP data

Diagnosis and Treatment of the Disease

Use of experimental methods that measure biological components on the molecular level is become starting to become more prevalent. Researchers are pushing to use microarrays, DNA sequencing, use of mass spec and other methods to measure protein levels in patient tissue, in-vivo tagging and measuring of proteins, etc. Starting from a simulation of a human cell, populations of cells, tissues, organs, and eventually the human level, one can input this patient specific data into the simulation and optimize or train the models to the patient specific data. Once this is done, tailored treatments can be developed using the simulation to determine:

Proper diagnosis of the disease (which types of cancer do they have, what is the stage of the cancer, . . . etc.)

Optimal targets to perturb (single or combinations)

Optimal lead compound(s) and other therapies to use (single or combinations)

Optimal dose and therapeutic regimen

To test the therapy in silico before applying to the human

Example Applications Starting from an Optimized Model of Colon Cancer:

Starting from an optimized simulation of the pathways underlying cell growth, survival, and apoptosis, several predictions were made with direct application to drug discovery and development. FIG. 31 shows a modular schematic description of the cell growth, survival, and death receptor signals that feed into the main apoptosis module. This apoptosis module, is the core module that controls whether the cell will undergo apoptosis or not. Cross talk coming from the cell growth signals of the Ras MapK Module, the IGF-1/P13K Modle, and the NF-kB Module inhibit apoptosis. While cross talk coming from the Death Receptor Modules both promote the onset of apoptosis via proteolitic cleavage of caspase-8 and activation of the NF-kB module. The FKHR Module and the p53 Module both promote apoptosis when activated. The onset of apoptosis is mainly determined by the balance of pro-apoptotic proteins in the cell such as Bax and Bad and anti-apoptotic proteins such as Bcl-2, Bcl-xL, XIAP, and FLIP and the dynamics of activating the various caspases, caspase-8, -9, and -3.

FIG. 32 shows the detailed DCL model of that pathway which can be directly translated to a set of reactions and solved in the appropriate manner to generate simulation output as previously discussed. The model was trained and optimized to the data sets on Trail, TNF, and Fas stimulation as well as EGF and IGF-1 stimulation. In addition, the specific genetic pathology of the cancer and cell type interested in deriving predictions on was inputted into the simulation and optimization. For example, the Bax gene is mutated on one aleel and thus concentrations are half of what is in a normal cell. The component Ras MapK is mutated and thus the parameter controlling the hydrolysis rate is reduced. Levels of Bcl-2 and Bad are similar while levels of Bcl-xL are a factor of two higher. In this way, a simulation can be trained to any genetic pathology reflective of the type of cancer type or even the cancer in a specific patient (maybe show this within the context of the reaction steps and the actual values).

The main results of the data, is that TNF and FAS (antibody) weakly induce apoptosis in the cell because they have low receptor numbers and the activation of NFkB is able to thwart apoptosis; while Trail induces strong apoptosis at concentrations of 2 ng/ml and higher because there are plenty of trail receptors on the cell surface. At 0.2 ng/ml of Trail, the three cytokines induce the same amount of cell death as shown in the experimental measurement of cell death under Trail, TNF, and Fas stimulation.

Importance of Receptor Numbers for Determining Sensitivity to Trail, TNF, and FAS Treatments.

From the optimized simulation, a cell with high numbers of Trail receptors, TrailR=35,000 receptors/cell, was simulated under the condition of adding 2 ng/ml of Trail (ligand not receptor). FIG. 33 shows the simulation output where caspase-8 is activated within the first few minutes (activation of all of the caspases occurs via proteolitic, caspase-8 cleave occurs via the Trail receptor death complex). This then leads to the cascade of activating Bid, Bax, and disruption of mitochondrial integrity whereby caspase-9 and caspase-3 are activated and Parp is cleaved. The activation of caspase-3 is the chemical signature that the cell is undergoing apoptosis. In order for cell death to occur, caspase-3 has to be activated close to its maximal levels. The timing of and strength of caspase-3 activation experimentally correlates with the induction of cell death (the faster and stronger the sooner). Under 2 ng/ml Trail the cells die by 24 hours.

Taking the same optimized simulation Trail Receptors numbers were decreased from 35,000 receptors/cell to 3,000 receptors/cell. The model then predicts that the timing of caspase activation is shifted by 500 minutes. This would then mean that the onset of cell death is not going to occur until well after 48 hours. The model predicts that cell surface receptor numbers are a good indicator of the sensitivity the cell towards Trail induction of apoptosis. Cells with low numbers of Trail will not be responsive and cells with a high numbers of Trail receptors will. The model predicts something similar for TNF and FAS stimulation. An experimental assay can therefore be set up that directly measures Trail, FAS, and TNF receptor numbers to determine which cell types and cancer types will undergo cell death as a result of stimulating with those three targets.

The simulation let to insight as to the biological role of receptor numbers in inducing apoptosis and led to the discovery of a molecular marker that can be predictive of a cell's responsiveness to a given therapy.

Target Predictions

Predicting the synergistic single or combination perturbation in a simulation can occur in a number of ways.

The first is via examining the network diagram underlying the circuitry such as the one in FIG. 32. Here one can find nodes that appear to be the most lethal based on their connections to pro-apoptotic proteins in the apoptosis module.

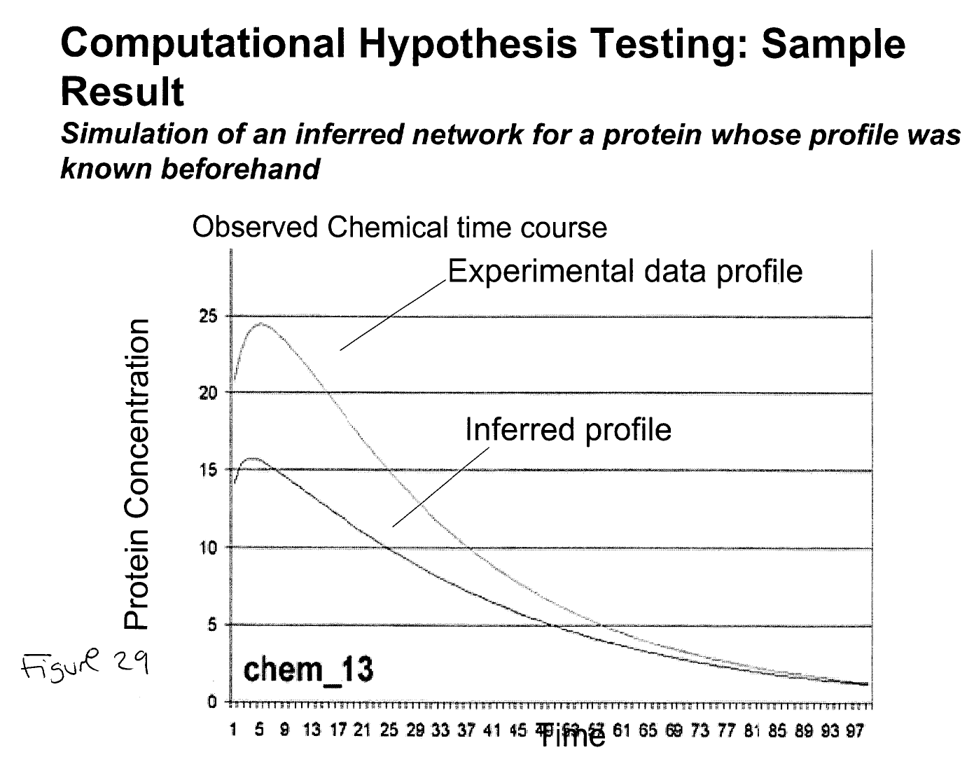

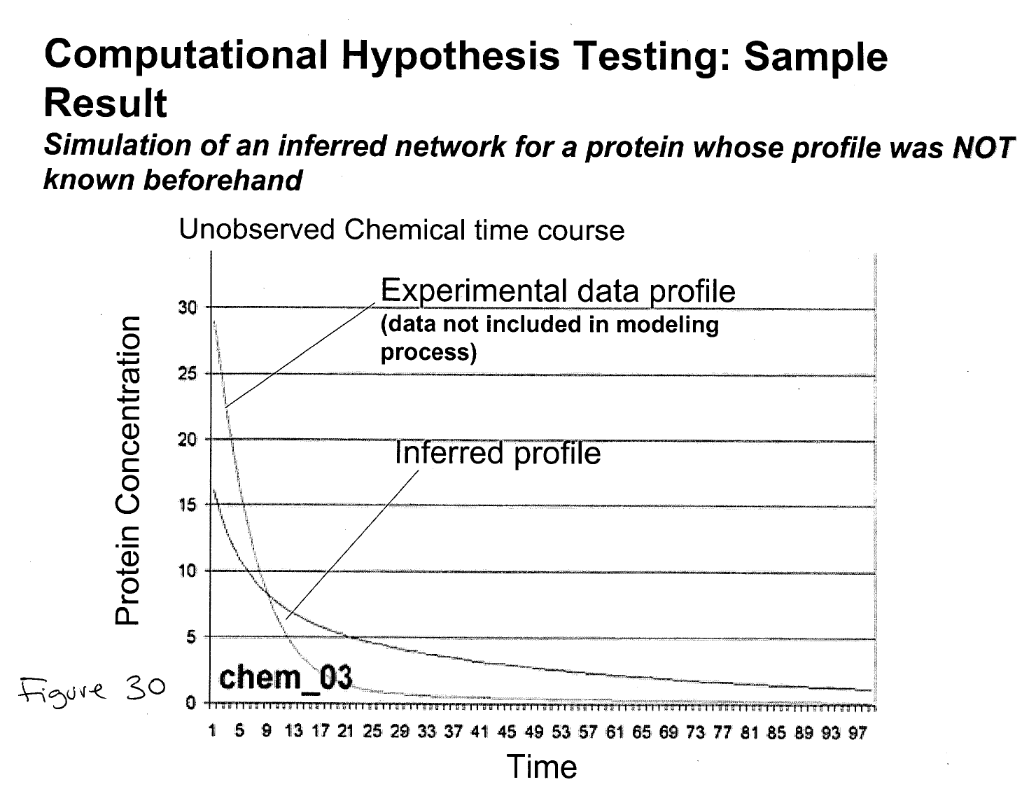

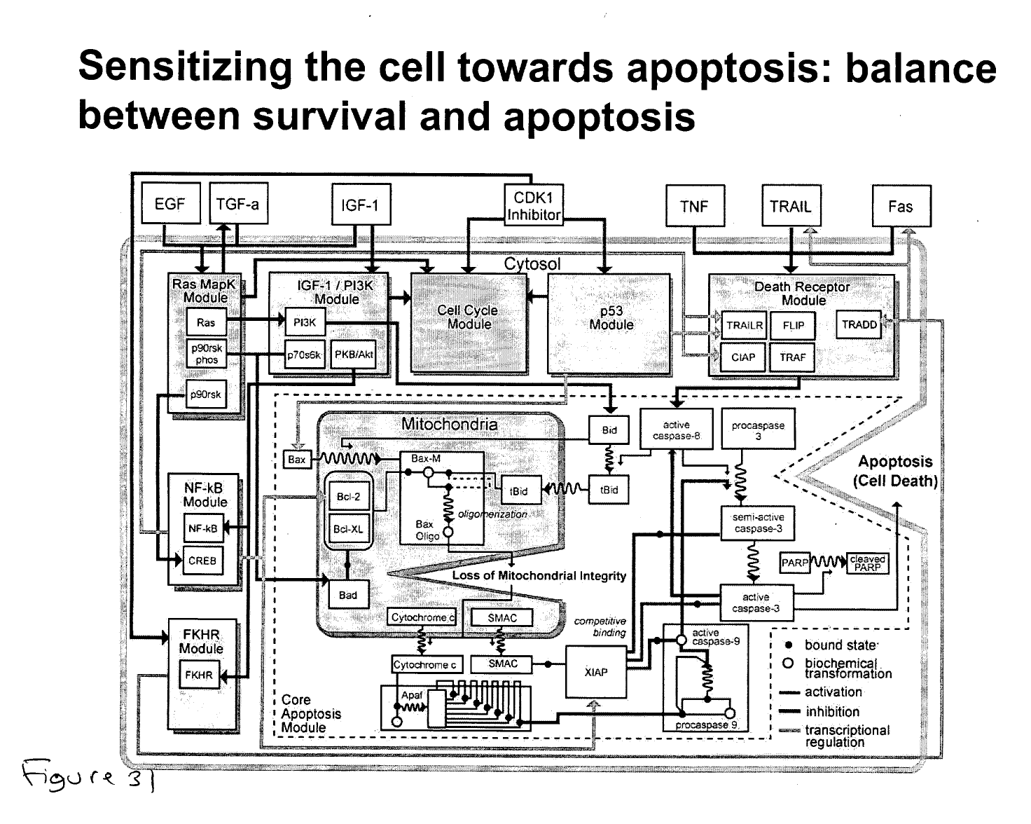

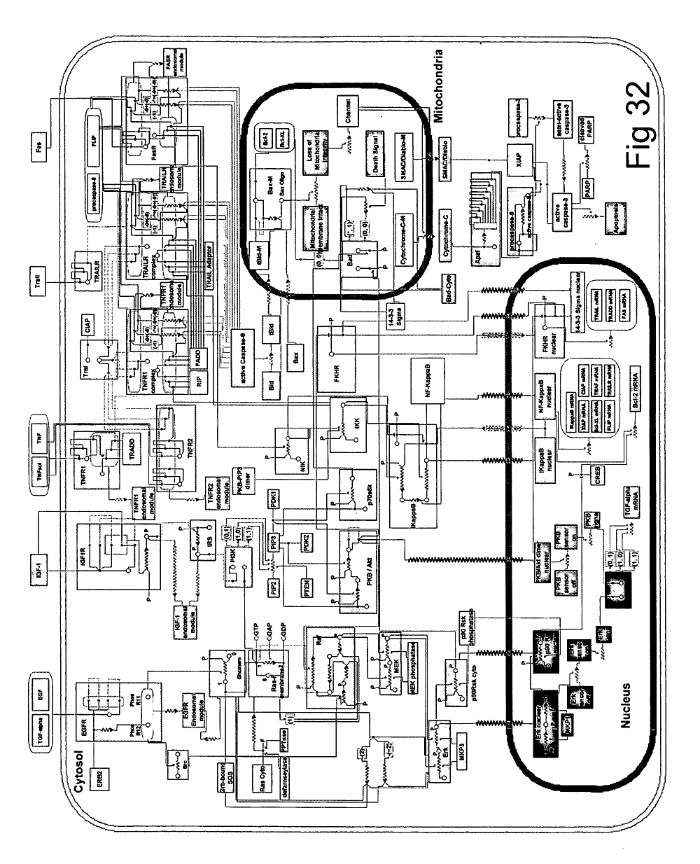

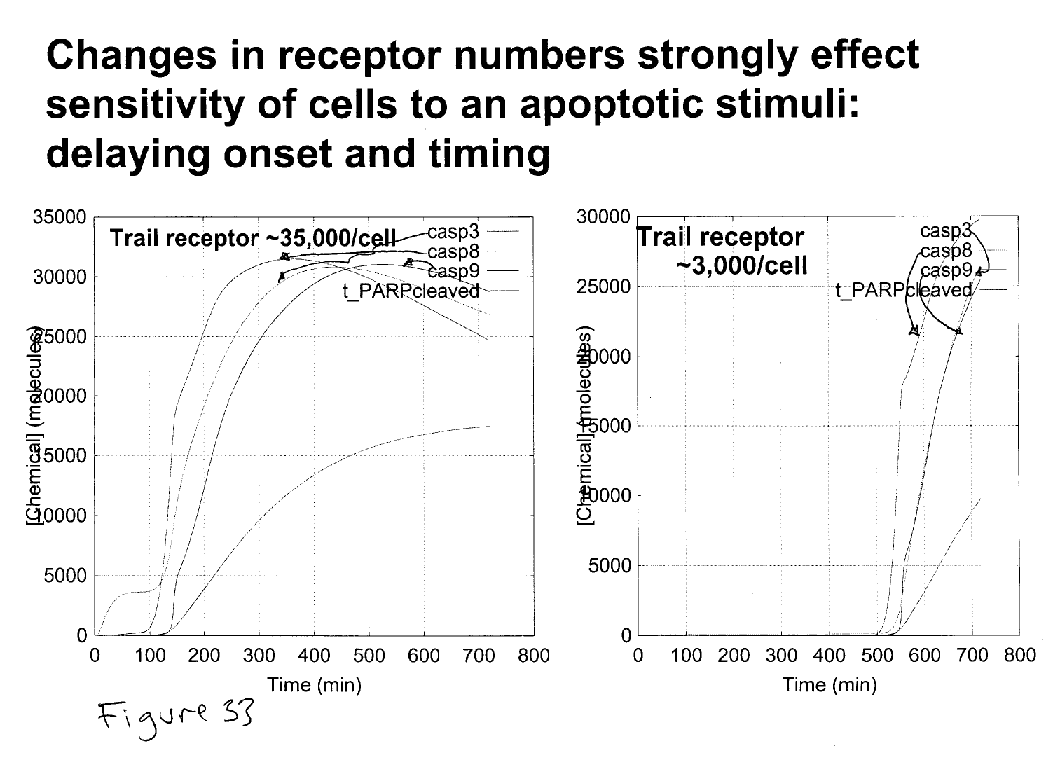

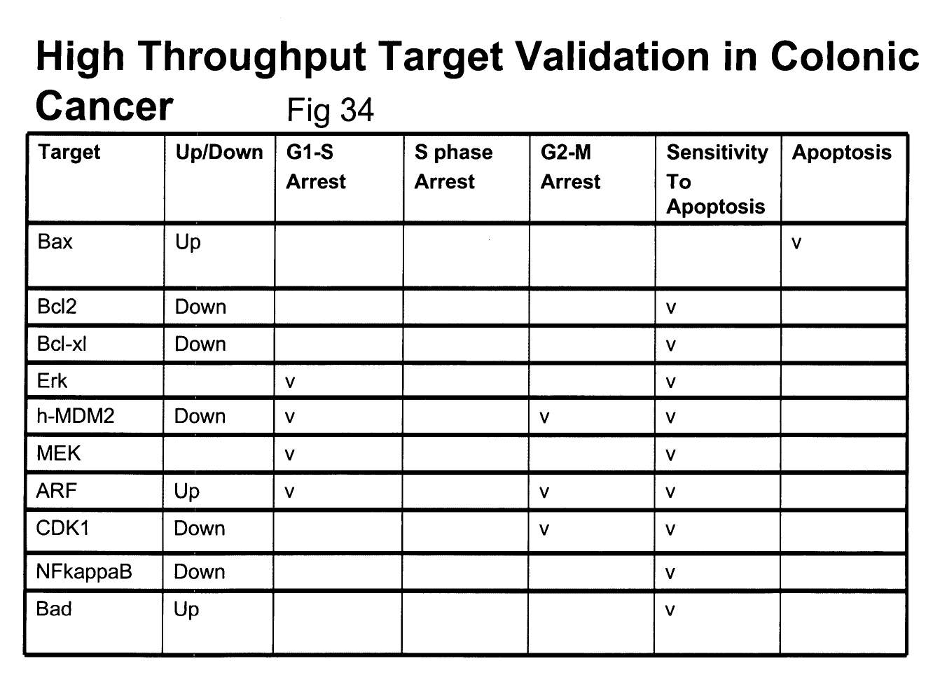

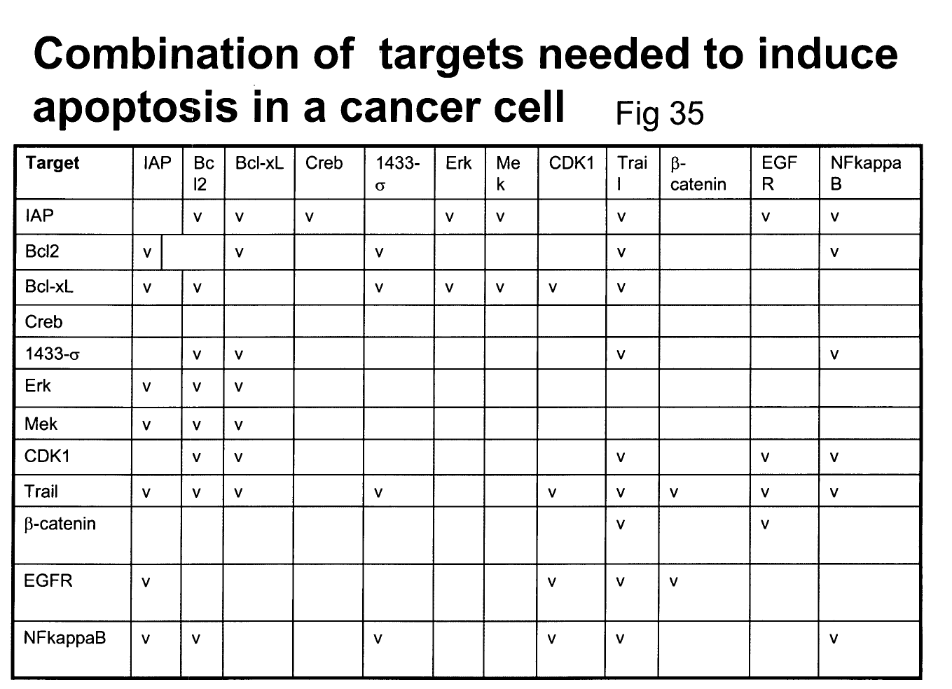

The second is via an algorithmic code that automatically knocks out the single targets and combinations there of and checks the chemical signature of the physiological state one wishes to test. For example, the code could have the form for all targets i through N set the chemical concentrations and over levels to zero. Then check that caspase-3 activity reaches its maximum by 4 hours. These are the targets that when knocked out singly lead to significant cell death. Then one can loop through j through M to when a secondary target is knocked out in combination with the ith target and the same check performed. The i,j combination that leads to significant caspase activity is the synergistic combination. This again, can be done with target knockouts, addition of inhibitors, and other components such as cytokines. An example of the output from single target knockouts and combination target knockouts is shown in FIGS. 34 and 35.

The third is via manual human testing and intervention with the model whereby one learns from the model and learns of key concepts that can give the researcher hints of where and how to perturb. Following on the example of the Trail receptor modulation, one learns that therapeutics that promote the increase of death receptors on cell surface can promote apoptosis and are worth pursuing.

Predicting Synergistic Drug Target Combinations

Often the goal of cancer therapy is to find single or combination targets that synergistically would lead to apoptosis in cancer cells. Many of the targeted therapies currently in clinical trials, alone, are not enough to induce strong apoptosis in cancer cells. In this case we are referring to strong apoptosis as any perturbation that is predicted to lead to significant cell death before 24 hours such as doses of Trail of 2 ng/ml and higher.

Two targets of interest are CDK1 and P13K. CDK1 has a lead compound called—that is in Phase I clinical trials. The model predicts that inhibiting CDK1 or P13K will not lead to significant apoptosis before 24 hours. The model and experiment also-predict that the three cytokines Trail at 0.2 ng/ml, TNF, and Fas alone all will not lead to significant apoptosis as well prior to 24 hours. The question asked of the simulation was, which two-way combinations of the CDK1 or P13K and one of the three cytokines would (see FIG. 36)? To generate the results, the CDK1 inhibitor was inputted into the simulation. The mechanism of action of the inhibitor occurs via:

Activation of p53 which up regulates total Bax levels and Trail receptor numbers

Non-specifcity of the inhibitor whereby it inhibits GSK3 and leads to FKHR up regulation and accumulation of Trail ligand

These specific interactions were inputted in the simulation, either via DCL interface or directly into the simulation engine with reaction forms like:

The CDK1 inhibitor was added at t=0. At t=24 hours, the cytokine treatments were added in singleton combinations via a function in the digital cell called a “Switch” which is zero for t<24 hours and 1 for t>24 hours. The model then predicts that only CDK1 and 0.2 ng/ml of Trail synergistically induce apoptosis, while TNF or FAS do not.

To generate the results on inhibiting P13K, P13K was simply knocked out or its concentrations and levels set to zero. The effect of this in the simulation is to:

Increase in active Bad levels: inactivated by phosphorylation

Increase in active Bid levels: inactivated by phosphorylation

Down regulation of NFkB transcription

Up-regulation of Trail ligand via FKHR nuclear translocation

P13K was knock out at t=0 and t=24 hours the cytokines were added in singleton combinations using the “Switch” function again. The model predicts all three cytokines will be responsive, but the amount depends on the effect of each cytokine alone

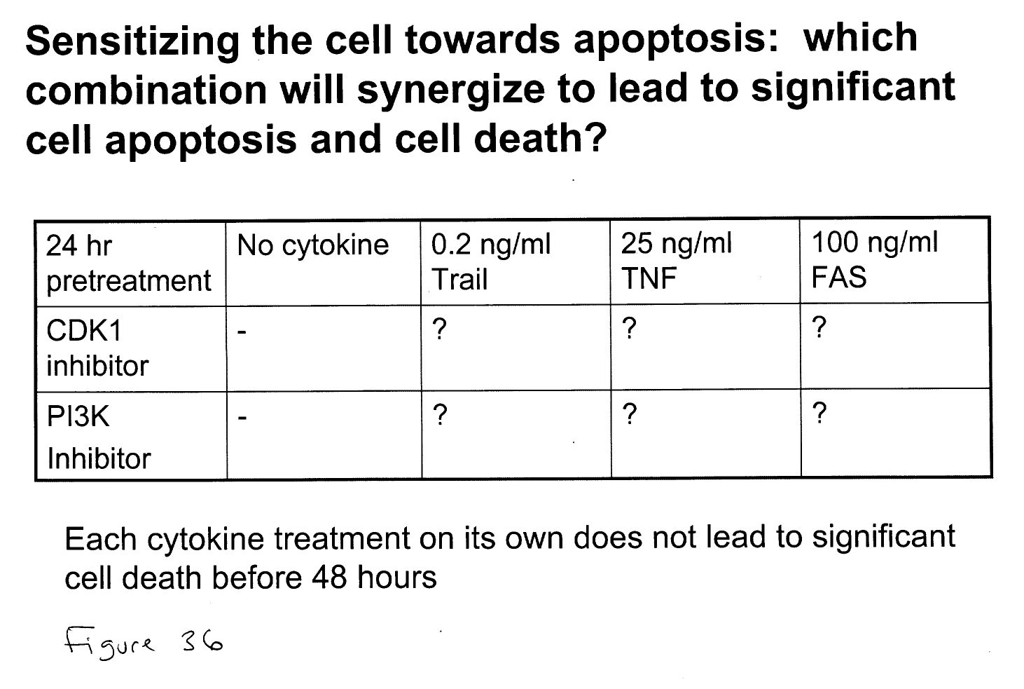

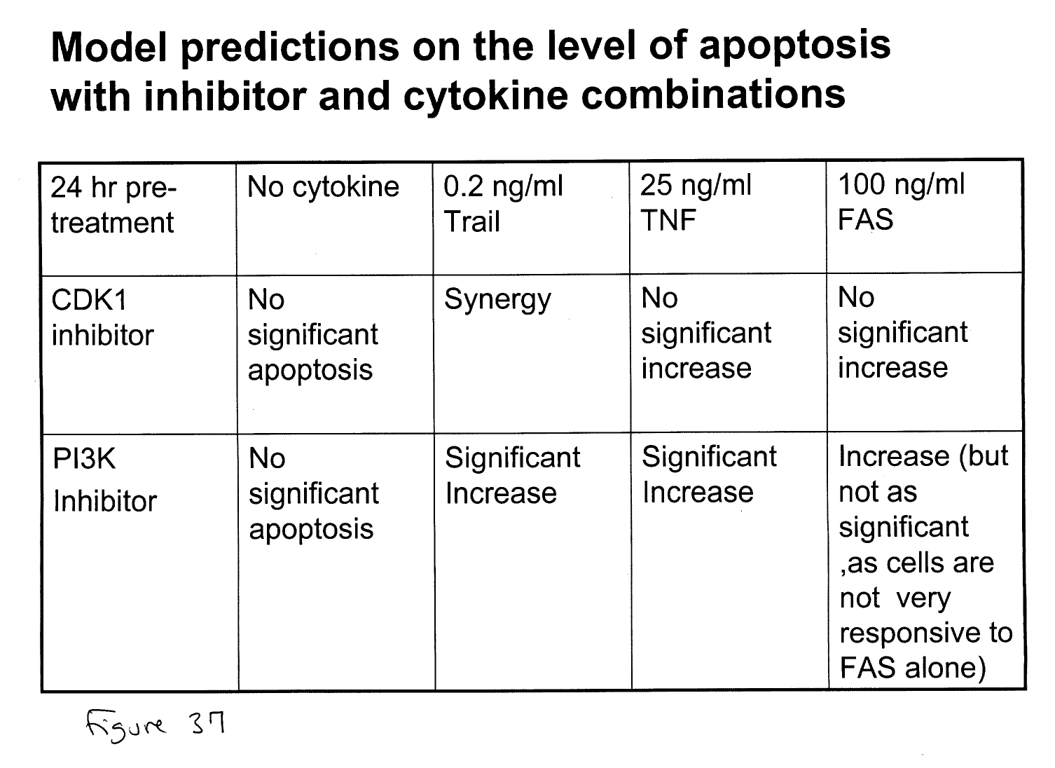

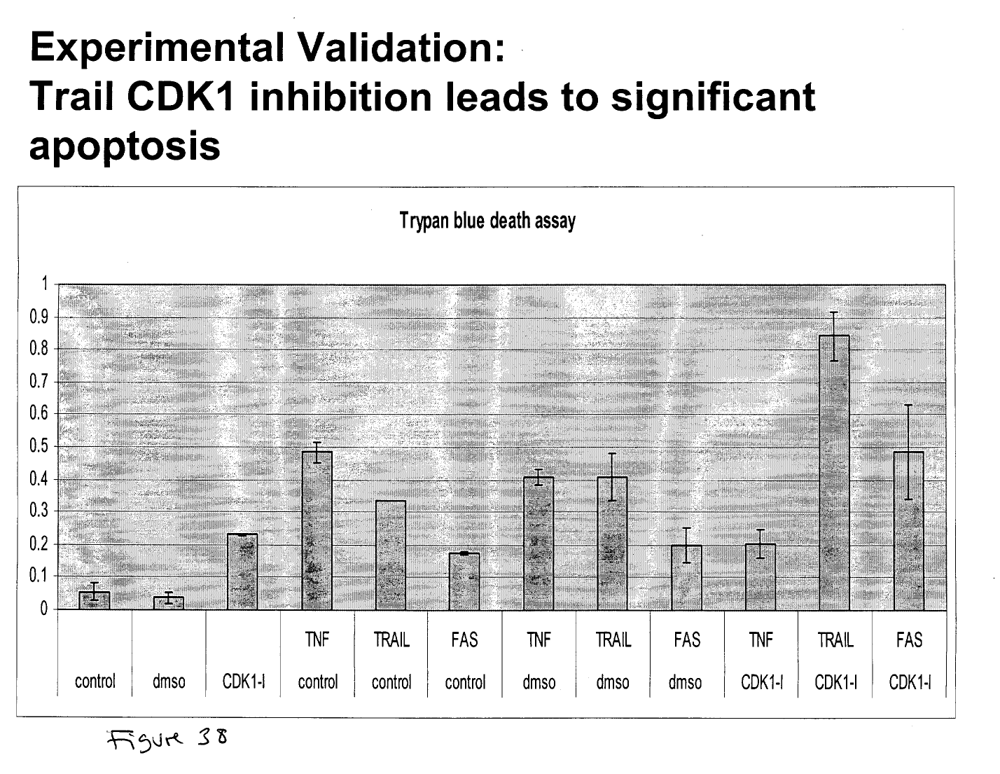

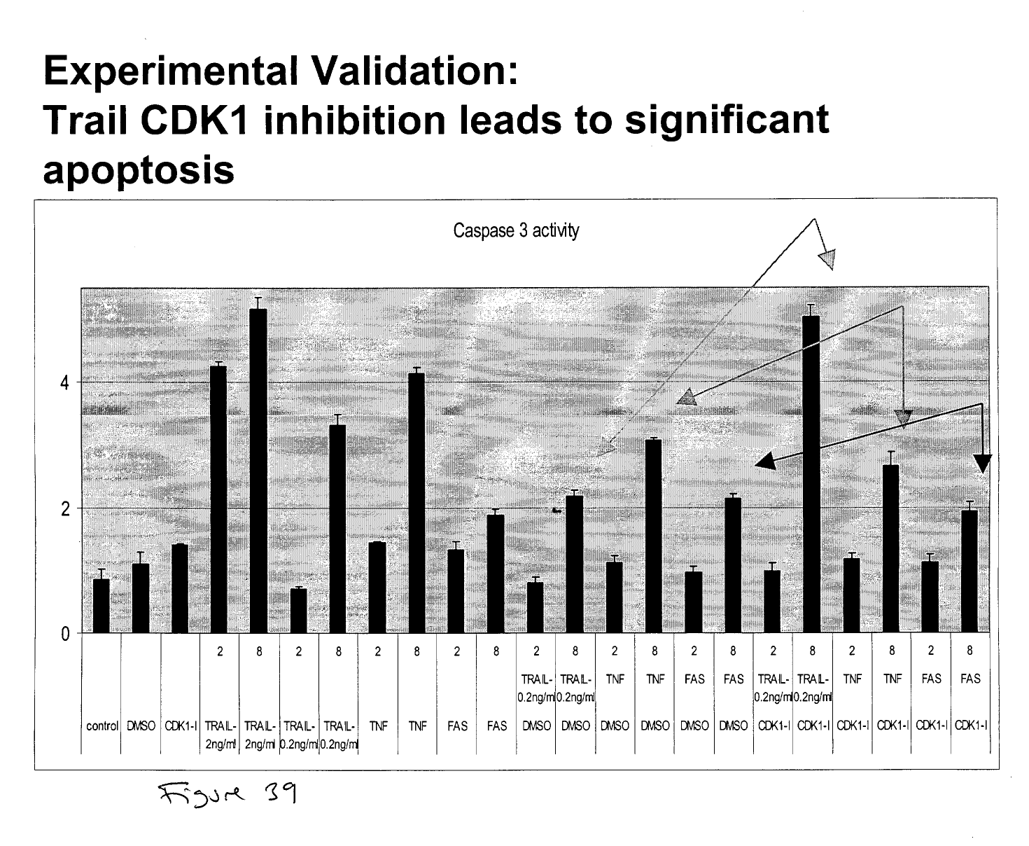

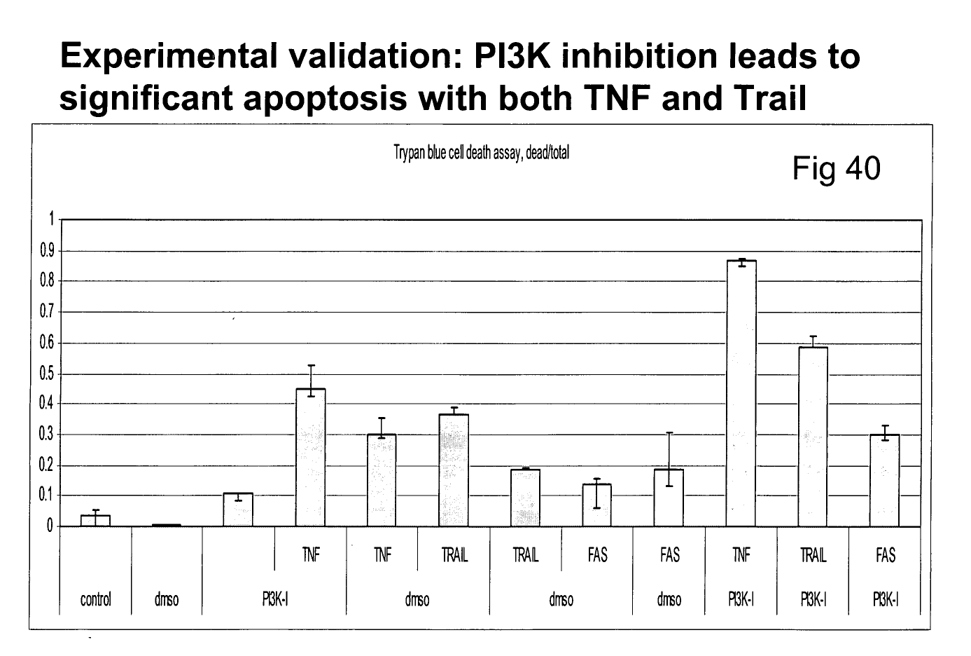

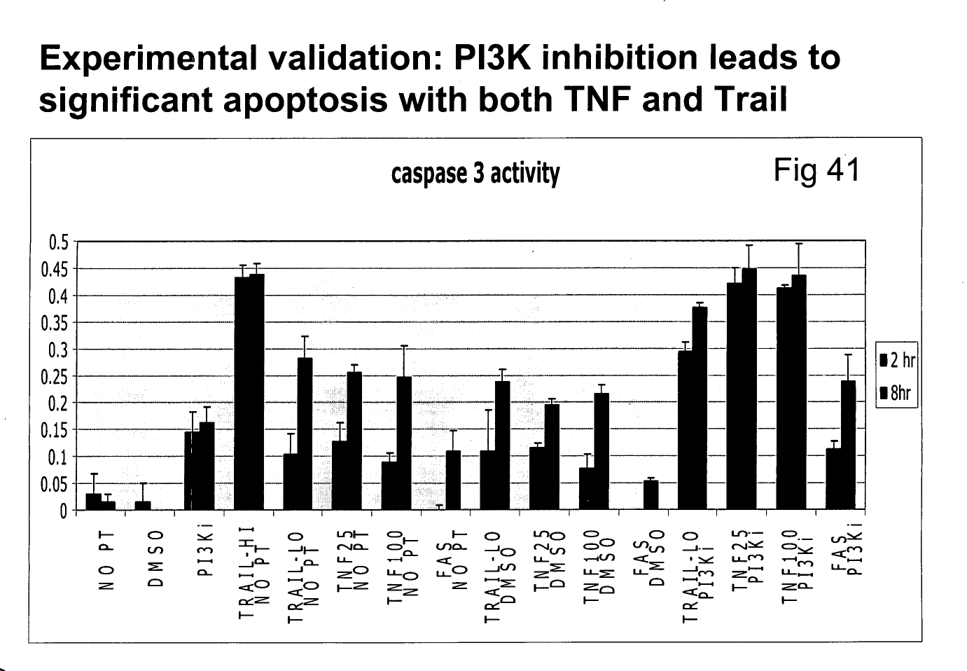

FIG. 37 summarizes the results of the in silico perturbations. Taking the methodology full circle where the results were verified experimentally. This is shown in FIGS. 38-41 where the cell death experiments were confirmed by caspase-3 activity assays.

Optimization of Bioreactor/Fermentation Conditions

A bacterial whole cell simulation can be used to predict outcome of exposure to changes in external environment conditions. The model can be used to simulate growth of microbes in industrial fermentation reactors and analyze the effect of either internal genetic modifications or external culture changes on production of end product metabolites (amino acids, vitamins, production of recombinant proteins, antibodies, etc). The whole cell bacterial simulation can also serve as a template for building models of populations of bacterial cells in the bioreactor. Applications would include optimization of industrial mircobiology processes including reactor conditions and strain optimization. The applications described here extend to other cell types and or biological systems where once is interested in using the cell or biological system to produce various end products, for example, the use of Mammalian Hela Cells to create antibodies. The exemplary methodologies of the invention I-VI can then be used for the following:

(1) prediction of phenotypic: here one is interested in taking a bacteria, perturbing the cellular constituents of the bacteria to increase output of various end products such as amino acids, vitamins, production of recombinant proteins, etc.

(2) elucidating which components to measure in order to determine phenotypic outcome: one is interested in increasing production on an output product in a particular strain or strains of bacteria and is interested in knowing which components to measure under various test conditions to optimize production.

(3) discovering new biological pathways in the system: as there remains a good portion of most bacterial genomes with unknown functionality new pathways could provide new modes for perturbing the bacterial system. Similarly for (4) elucidating the function of unknown or poorly characterized genes and protein.

(5) elucidating the mechanism of action of entities used to perturb the system (such as a drug).

(6) tailoring 1-5 to match a particular genetic pathology of the cell or biological system: often in industry one takes the endogenous strain and genetically modifies it to create a particular output. The genetically modified strain then has a genetic pathology that is different than its endogenous counterpart. The simulation can be tweaked to reflect this genetic pathology. It can also be tweaked to determine the optimal genetic pathology that would give optimal production. The strain can then be modified to reflect this.

Using the E. Coli Model to Increase Lysine Production

The E. coli model is used to simulate lysine production under the normal physiological conditions. In this case 12 molecules of Lysine are produced as was shown in FIG. 22. The simulation is then perturbed by increasing the carbon flux through the pathway. When this is done, the number of Lysine molecules increases to 20 shown in FIG. 42. Exhibit contains the paramter values of the normal and perturbed conditions that lead to an increase in the carbon flux. A similar result could have been obtained via changing any of the parameters in the model including connectivity between the components.

Specifically the in silico simulation will help:

1. Quantitative increases in lysine production with gradual increases in concentrations of the supplied initial carbon source, glucose.

2. Quantitative increases in lysine production with selected levels of inactivation of the lysine sensitive aspartokinase isozyme LysC.

3. Evaluation of ways to channel less carbon flux through the methionine and threonine arms of the pathways, thus enabling more carbon flux through the lysine arm of the pathway.

4. Impact of replacing the wild type DapA with a less lysine-sensitive variant on end product production.

5. Impact of changing other unrelated proteins or genes on the lysine pathway.

C. Genetic Engineering: here one is interested in constructing a bacterium with certain functionality to be used for industrial production, bioremediation, . . . etc. An in silico simulation can be constructed to generate network topologies composed of genes, protein, metabolites, and other cellular constituents to give the desired functionality either by desiging an organism from scratch or starting from an existing organism. Detailed model of bacteria such as E. coli can be used to determine the minimum number of components needed to create a bacterial cell and what networks to add to that to engineer specialized bacterium.

In addition to the exemplary applications described thus far the methods and techniques of the present invention can be applied to obtain and improve results better than available in each of the following applications as well: infectious disease (finding lethal targets for the infectious bacteria while sparing the human), bioremediation, biodefense (finding lethal targets for pathogens and components to measure to detect pathogens), biofilm formation (on pacemakers, boats, etc., how to eliminate biofilms by targeting certain components), biosensors (determining which components to measure to sense particular organisms), etc.

As noted above in the description of the invention which has been provided herein, numerous references to exemplary embodiments, processes and techniques have been stated without language of exemplification. It is to be understood that any embodiment, technique, method, process or the like described herein is only exemplary in nature, and not meant to in any way limit, define or restrict the invention presented herein, which is greater than the sum of any one or more constitutent parts, processes or methods used to describe it. In addition, where forms of the verb “to be” are used to describe techniques, processes, results or methods, or parts thereof, what is intended and understood by such usage is actually the compound auxilliary verb formation “can be.” Thus, if a given technique “is” used or a given result is said to occur, what is understood and intended is that the technique “can be” used or “may be” used and the result can or may occur.

The description of the foregoing invention is merely exemplary in nature. The scope of the invention is to be defined by the following claims.

What is claimed:

1. A method of creating a scalable simulation of a biological system, comprising:
extracting data from the vast body of public domain information;
rating the biological information to determine the quality of the data;
representing the information in a concise and scalable manner that is automatically translatable to a mathematical representation or set of instructions readable by a human or computer; and
integrating diverse data types to account for missing biological information and to infer the functionality of unknown biological components and their interactions.

2. The method of claim 1 where the information is represented via a concise and scaleable language for representing and simulating biological interactions.

3. The method of claim 2, where the language comprises the Diagrammatic Cell Language.

4. The method of claim 1, where said unkown biological components comprise at least one of genes, proteins, and metabolites.

5. The method of claim 1 where integrating diverse data types includes utilizing data mining tools.
